SEQUENCE LISTING (52 sequences)

SEQ ID NO: 1 - DNA, 654 bp, Artificial Sequence
  Description of Artificial Sequence  note = Synthetic Construct
dtgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattgtacgccctgtccgccgtcggccacttcaccctgggctgtgtgctggaggaccaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcggcggaggcggaggcagtccgcgcgccgatcccaaaaagaaaagaaaggtagcagccatggcctaa

SEQ ID NO: 2 - DNA, 891 bp, Artificial Sequence
  Description of Artificial Sequence  note = Synthetic Construct
datggccagccgcctggacaagtccaaggtcatcaattccgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattgtacgccctgtccgccgtcggccacttcaccctgggctgtgtgctggaggaccaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcggcggaggcggaggcagtccgcgcgccgatcccaaaaagaaaagaaaggtagcacgcgtcggcggaggcggaagtgggtccccggccgacgccctggacgacttcgacctggacatgctgccggccgacgccctggacgacttcgacctggacatgctgccggccgacgccctggacgacttcgacctggacatgctgccggccgacgccctggacgacttcgacctggacatgctgccggggtaactaagtaa

SEQ ID NO: 3 - DNA, 891 bp, Artificial Sequence
  Description of Artificial Sequence  note = Synthetic Construct
datggccagccgcctggacaagtccaaggtcatcaatggcgccctggagctgctgaacggcgtcggaatcgaaggtttaacaacccgtaaactcgcccagaagctaggtgtagagcagcctacattgtattggcatgtaaaaaataagcgggctttgctcgacgccttacccatcgagatgctggaccgccaccacacccacttctgccccctggagggcgagagctggcaggacttcttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattgtacgccctgtccgccgtcggccacttcaccctgggctgtgtgctggaggagcaggagcatcaagtcgctaaagaagaaagggaaacacctactactgatagtatgccgccattattacgacaagctatcgaattatttgatcgccaaggcgccgagcccgccttcctgttcggcctggagctgatcatctgcggcctggagaagcagctgaagtgcgagagcggcagcgcctacagccgcggcggaggcggaggcagtccgcgcgccgatcccaaaaagaaaagaaaggtagcacgcgtcggcggaggcggaagtgggtccccggccgacgccctggacgacttcgacctggacatgctgccggccgacgccctggacgacttcgacctggacatgctgccggccgacgccctggacgacttcgacctggacatgctgccggccgacgccctggacgacttcgacctggacatgctgccggggtaactaagtaa

SEQ ID NO: 4 - DNA, 901 bp, Artificial Sequence
  Description of Artificial Sequence  note = Synthetic Construct
datggcctccagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctagccccccccagtttgaacaagcagaggcgcaactacgctgtctatgcgatgcagggttttcggtcgaggaggctcttttcattctgcaatctataagccattttagcttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaggcggaggcggagggcgcgccgatcccaaaaagaaaagaaaggtagcacgcgccgggggaggcggcctggcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggacccctggtaa

SEQ ID NO: 5 - DNA, 1000 bp, Artificial Sequence
  Description of Artificial Sequence  note = Synthetic Construct
datggcctccagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctagccccccccagtttgaacaagcagaggcgcaactacgctgtctatgcgatgcagggttttcggtcgaggaggctcttttcattctgcaatctataagccattttagcttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaggcggaggcggagggcgcgccgatcccaaaaagaaaagaaaggtagcacgcgccgggggaggcggcctgatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaa

SEQ ID NO: 6 - DNA, 107 bp, Artificial Sequence
  Description of Artificial Sequence  note = Synthetic Construct
dactagtcatgcaaattacgcgctgtgctttgtgggaaatcaccctaaacgtaaaatccctatcagtgatagagacttataatccctatcagtgatagagaggatcc

SEQ ID NO: 7 - DNA, 8 bp, Artificial Sequence
  Description of Artificial Sequence  note = Synthetic Construct
ruuuuuua

SEQ ID NO: 8 - DNA, 7 bp, Artificial Sequence
  Description of Artificial Sequence  note = Synthetic Construct
ruuuuua

SEQ ID NO: 9 - DNA, 7 bp, Artificial Sequence
  Description of Artificial Sequence  note = Synthetic Construct
ruuuuuu

SEQ ID NO: 10 - DNA, 104 bp, Artificial Sequence
  Description of Artificial Sequence  note = Synthetic Construct
dggaaaggaaggacaccaaatgaaagattgtactgagagacaggctaatttcctgggcaaaatttggccaagtcacaagggaaggccagggaattttcttcaga

SEQ ID NO: 11 - DNA, 105 bp, Artificial Sequence
  Description of Artificial Sequence  note = Synthetic Construct
daggctaacttcttcagggaagatctggcatttccgcagggtaaagcgcgtgaattttcctcagagcagaccagagccaacagccccaccagaagagagcttcag

SEQ ID NO: 12 - DNA, 1878 bp, Artificial Sequence
  Description of Artificial Sequence  note = Synthetic Construct
datctctatcactgatagggagatctctatcactgatagggagagctctgcttatatagacctcccaccgtacacgcctaccgcccatttgcgtcaatggggcggagttgttacgacattttggaaagtcccgttgattttggttccaaaacaaactcccattgacgtcaatggggtggagacttggaaatccccgtgagtcaaaccgctatccacgcccattgatgtactgccaaaaccgcatcaccatggtaatagcgatgactaatacaattctaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacagtagcttcca

SEQ ID NO: 13 - DNA, 1732 bp, Artificial Sequence
  Description of Artificial Sequence  note = Synthetic Construct
dggatccgatctctatcactgatagggagatctctatcactgatagggagagctctgcttatatagacctcccaccgtacacgcctaccgcccatttgcgtcaatggggcggagttgttacgacattttggaaagtcccgttgattttggttccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttagaattcaagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcaggtgtcgtgaggatctactag

SEQ ID NO: 14 - DNA, 1715 bp, Artificial Sequence
  Description of Artificial Sequence  note = Synthetic Construct
dggatcctctctatcactgatagggattataagtctctatcactgatagggattttacgtttagggtgatttcccacaaagcacagcgcgtaatttgcatgactagtcaattctaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacagtagcttcca

SEQ ID NO: 15 - DNA, 6 bp, Artificial Sequence
  Description of Artificial Sequence  note = Synthetic Construct
dggggs

SEQ ID NO: 16 - DNA, 21 bp, Artificial Sequence
  Description of Artificial Sequence  note = Synthetic Construct
dtcgaaggtttaacaacccgt

SEQ ID NO: 17 - DNA, 21 bp, Artificial Sequence
  Description of Artificial Sequence  note = Synthetic Construct
dttgtcgtaataatggcggca

SEQ ID NO: 18 - DNA, 34 bp, Artificial Sequence
  Description of Artificial Sequence  note = Synthetic Construct
dgcggccgcaattcatatttgcatgtcgctatgt

SEQ ID NO: 19 - DNA, 78 bp, Artificial Sequence
  Description of Artificial Sequence  note = Synthetic Construct
dgaattcgcggatcctctctatcactgatagggacttataagtctctatcactgatagggatttcacgtttatggtga

SEQ ID NO: 20 - DNA, 38 bp, Artificial Sequence
  Description of Artificial Sequence  note = Synthetic Construct
dggcggccgcatatgactagtcatgcaaattacgcgct

SEQ ID NO: 21 - DNA, 79 bp, Artificial Sequence
  Description of Artificial Sequence  note = Synthetic Construct
dgaattctggatcctctctatcactgatagggattataagtctctatcactgatagggattttacgtttagggtgattt

SEQ ID NO: 22 - DNA, 61 bp, Artificial Sequence
  Description of Artificial Sequence  note = Synthetic Construct
dgatccagctgaccctgaagttcatcttcaagagagatgaacttcagggtcagctttttgg

SEQ ID NO: 23 - DNA, 61 bp, Artificial Sequence
  Description of Artificial Sequence  note = Synthetic Construct
daattccaaaaagctgaccctgaagttcatctctcttgaagatgaacttcagggtcagctg

SEQ ID NO: 24 - DNA, 65 bp, Artificial Sequence
  Description of Artificial Sequence  note = Synthetic Construct
dgatccaggatggtggtgtttcaattccttcaagagaggaattgaaacaccaccatcctttttgg

SEQ ID NO: 25 - DNA, 65 bp, Artificial Sequence
  Description of Artificial Sequence  note = Synthetic Construct
daattccaaaaaggatggtggtgtttcaattcctctcttgaaggaattgaaacaccaccatcctg

SEQ ID NO: 26 - DNA, 22 bp, Artificial Sequence
  Description of Artificial Sequence  note = Synthetic Construct
dcggcatcaaggtgaacttcaa

SEQ ID NO: 27 - DNA, 55 bp, Artificial Sequence
  Description of Artificial Sequence  note = Synthetic Construct
dcgaattcgagctcggtacccgggatcgcgtgaagcgcgcacggcaagaggcgag

SEQ ID NO: 28 - DNA, 44 bp, Artificial Sequence
  Description of Artificial Sequence  note = Synthetic Construct
dcatgttggccaaattttgcccaggaaattagcctgtctctcag

SEQ ID NO: 29 - DNA, 28 bp, Artificial Sequence
  Description of Artificial Sequence  note = Synthetic Construct
dtttggccaagtcacaagggaaggccag

SEQ ID NO: 30 - DNA, 42 bp, Artificial Sequence
  Description of Artificial Sequence  note = Synthetic Construct
dctcgacatgacgcgttattgtgacgaggggtcgctgccaaa

SEQ ID NO: 31 - DNA, 45 bp, Artificial Sequence
  Description of Artificial Sequence  note = Synthetic Construct
dgaattcacgcgtatgggcgcgcgtgcgtcagtattgagcggggg

SEQ ID NO: 32 - DNA, 45 bp, Artificial Sequence
  Description of Artificial Sequence  note = Synthetic Construct
dcgcagatcttccctgaagaagttagcctgtctctcagtacaatc

SEQ ID NO: 33 - DNA, 63 bp, Artificial Sequence
  Description of Artificial Sequence  note = Synthetic Construct
dagatctggcatttccgcagggtaaagcgcgtgaattttcctcagagcagaccagagccaaca

SEQ ID NO: 34 - DNA, 47 bp, Artificial Sequence
  Description of Artificial Sequence  note = Synthetic Construct
dgcctcgagcgatgtcgacacccaattctgaaaagagtaaacagcag

SEQ ID NO: 35 - DNA, 35 bp, Artificial Sequence
  Description of Artificial Sequence  note = Synthetic Construct
dctgacgacgcgtgccagccccctgatggggcgac

SEQ ID NO: 36 - DNA, 46 bp, Artificial Sequence
  Description of Artificial Sequence  note = Synthetic Construct
dcgcacgcgcgcccatggtgcgctgtgtacgagacctcccggggca

SEQ ID NO: 37 - DNA, 9416 bp, Artificial Sequence
  Description of Artificial Sequence  note = Synthetic Construct
dttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggaagctttagacaagatagaggaagagcaaaacaaaagtaagaccaccgcacagcaggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttggaattgcgcgttacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttggtttctcgacgctagcggtaccacgcgttacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagtgcggccagatctgggccatttgttccatgtgagtgctagtaacaggccttgtgtcctgttgaagttcactgatgccggtcagtcagtggccaaaaccggcatcaaggtgaacttcaacagcatacagccttcagcaagcctccaggatccggatccggatggcgtctccaggcgatctgacggttcactaaacgagctctgcttatataggcctcccaccgtacacgcctactcgacccgggtaccgagctcggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacgtcagggtcgataatcaagaattcgaattccggcggccgcgtctcaagggcatcggtcgactctagagggacagcccccccccaaagcccccagggatgtaattacgtccctcccccgctaggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaagcctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggcactagtcatatgaagccgaattcaattctaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacagtagcttccaatggccagccgcctggacaagtccaaggtcatcaatggcgccctggagctgctgaacggcgtcggaatcgaaggtttaacaacccgtaaactcgcccagaagctaggtgtagagcagcctacattgtattggcatgtaaaaaataagcgggctttgctcgacgccttacccatcgagatgctggaccgccaccacacccacttctgccccctggagggcgagagctggcaggacttcttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattgtacgccctgtccgccgtcggccacttcaccctgggctgtgtgctggaggagcaggagcatcaagtcgctaaagaagaaagggaaacacctactactgatagtatgccgccattattacgacaagctatcgaattatttgatcgccaaggcgccgagcccgccttcctgttcggcctggagctgatcatctgcggcctggagaagcagctgaagtgcgagagcggcagcgcctacagccgcggcggaggcggaggcagtccgcgcgccgatcccaaaaagaaaagaaaggtagcacgcgtcggcggaggcggaagtgggtccccggccgacgccctggacgacttcgacctggacatgctgccggccgacgccctggacgacttcgacctggacatgctgccggccgacgccctggacgacttcgacctggacatgctgccggccgacgccctggacgacttcgacctggacatgctgccggggtaactaagtaatttccctctagcgggatcaattccgccccccccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagttatctatatggccaagaagcccgtgcagctgcccggctactactacgtggactccaagctggacatcacctcccacaacgaggactacaccatcgtggagcagtacgagcgcgccgagggccgccaccacctgttcctgtagtcgacgtcgacgtcaccgccgacgtcgaggtgcccgaaggaccgcgcacctggtgcatgacccgcaagcccggtgcctgacgcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggtacctttaagaccaatgacttacaaggcagctgtagatcttagccactttttaaaagaaaaggggggactggaagggctaattcactcccaacgaagacaagatctgctttttgcttgtacggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 38 - DNA, 9396 bp, Artificial Sequence
  Description of Artificial Sequence  note = Synthetic Construct
dgtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccgggatcgcgtgaagcgcgcacggcaagaggcgaggggcggcgactggtgagagatgggtgcgagagcgtcagtattgagcgggggaaaattggataagtgggagaaaattcggttaaggccagggggaaagaaaaaatataaattaaaacatctagtatgggcaagcagggagctagaacgattcgcagttaatcccggcctgttagaaacagcagaaggctgtagacaaatactgggacagctacaaccgtcccttcagacaggatcagaagaacttaaatcattatataatacaatagcagtcctctattgtgtgcatcaaatgatagatgtaaaagacaccaaggaagctttagagaagatagaggaagagcaaaacaacagtaagaaaaaagcacagcaagcagcagctgacacaggaaacagcagccaggtcagccgaaattaccctatagtgcagaacatccaggggcaaatggtacatcaggccatatcacccagaactttaaatgcatgggtaaaagtagtagaagagaaggctttcagcccagaagtaatacccatgttttcagcattatcagaaggagccaccccacaagatttaaacaccatgctaaacacagtggggggacatcaagcagctatgcaaatgttaaaagagaccatcaatgaggaagctgcagaatgggatagattgcatccagtgcaagcagggcctgttgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaatttcctgggcaaaatttggccaagtcacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaacgcgtgccagccccctgatggggcgacactccaccatagatcaccatagatcactcccctgtgaggaactactgtcttcacgcagaaagcgtctagccatggcgtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtacacagcgcaccatgggcgcgcgtgcgtcagtattgagcgggggaaaattggataagtgggagaaaattcggttaaggccagggggaaagaaaaaatataaattaaaacatctagtatgggcaagcagggagctagaacgattcgcagttaatcccggcctgttagaaacagcagaaggctgtagacaaatactgggacagctacaaccgtcccttcagacaggatcagaagaacttaaatcattatataatacaatagcagtcctctattgtgtgcatcaaatgatagatgtaaaagacaccaaggaagctttagagaagatagaggaagagcaaaacaacagtaagaaaaaagcacagcaagcagcagctgacacaggaaacagcagccaggtcagccgaaattaccctatagtgcagaacatccaggggcaaatggtacatcaggccatatcacccagaactttaaatgcatgggtaaaagtagtagaagagaaggctttcagcccagaagtaatacccatgttttcagcattatcagaaggagccaccccacaagatttaaacaccatgctaaacacagtggggggacatcaagcagctatgcaaatgttaaaagagaccatcaatgaggaagctgcagaatgggatagattgcatccagtgcaagcagggcctgttgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaacttcttcagggaagatctggcatttccgcagggtaaagcgcgtgaattttcctcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaggacagtatgatcagatacccatagaaatctgtggacataaagctataggtacagtattagtaggacctacacctgtcaacataattggaagaaatctgttgactcagattggttgcactctaaattttccgattagtcctattgaaactgtaccagtaaaattaaagcccgggatggatggtccgaaagttaaacaatggccattgacagaagaaaaaataaaagcattagtagaaatttgtacagaaatggaaaaggaagggaagatttcaaaaattgggcctgaaaatccatacaatactccagtatttgctataaagaaaaaagacagtactaaatggagaaaattagtagatttcagagaacttaataagaggactcaagacttctgggaagttcaattaggaataccacatcccgctggattaaaaaagaaaaaatcagtaacagtactagatgtgggtgatcgctatttctcagttcccttagataaagacttcaggaaatatactgcatttaccatacctagtataaacaatgagacaccagggattagatatcagtacaatgtgctcccacagggatggaaaggatcaccagcaatattccaaagtagcatgacaaaaatcttagagccttttagaaagcaaaatccagacatagttatctatcagtacatggatgatttgtatgtaggatctgacttagaaatagggcagcatagaacaaaaatagaggaactgagacaacatctgttaaggtggggatttaccacaccagacaaaaaacatcagaaagaacctccattcctttggatgggttatgaactccatcctgataaatggacagtacagcctatagtgctgccagaaaaagacagctggactgtcaatgacatacagaagttagtgggaaaattgaattgggcaagtcagatttactcagggatcaaagtgaagcagttatgtaaactccttaggggaaccaaagcactaacagaagtagtaacactaacagaagaagcagagctagaactggcagaaaacagggaaattctaaaagaaccagtacatggagtgtattatgacccatcaaaagacttaatagcagaaatacagaaacaggggcaaggccaatggacatatcaaatttatcaagagccatttaaaaatctgaaaacagcaaaatatgcaagaacgaggggtgcccacactaatgatgtaaaacaattaacagaggcagtgcaaaaaataaccacagaatgcataataatatggggaaaaactcctaaatttagactgcccatacaaaaagaaacatgggaaacatggtggacagagtattggcaagccacctggattcctgaatgggagtttgtcaatacccctcccttagtgaaattatggtaccagttagagaaagagcccatagaaggcgcagaaactttctatgtagatggagcagctaacagggagactaaattaggaaaagcaggatatgttactaacaaaggaagacaaaaagttgtcaccctaactgacacaacaaatcagaagactgagttagaagcaattcatctagctttgcaggattctggattagaagtaaacatagtaacagactcacaatatgcattaggaatcattcaagcacaaccagataaaagtgaatcagaattagtcagtcaaataatagagcagttaataaaaaaggaaaaggtctacctggcatgggtaccagcacacaaaggaattggaggaaatgaacaagtagataaattagtcagtgctggatccaggaaagtactatttttagatggaatagataaggcccaagaagaacatgagaaatatcacagtaattggagagccatggctagtgattttaacttaccacctgtagtagcaaaagaaatagtagccagctgtgataaatgtcagctaaaaggagaagccatgcatggacaagtagactgtagtccaggaatatggcaactagattgcacacatctagaaggaaaaattatcctggtggcggttcatgtagccagtggatatatagaagcagaagttattccagcagagacagggcaggaaacagcatactttctcttaaaattagcaggaagatggccagtaaaaacaatacatacagacaatggcagcaatttcaccagtaccacggttaaggccgcctgttggtgggcagggatcaagcaggaatttggcattccctacaatccccaaagtcaaggagtagtagaatctatgaataaagaattaaagaaaattataggacaggtaagagatcaggctgaacatcttaaaacagcagtacaaatggcagtatttatccacaattttaaaagaaaaggggggattggggggtacagtgcaggggaaagaatagtagacataatagcaacagacatacaaactaaagaactacaaaaacaaattacaaaaattcaaaattttcgggtttattacagggacaacaaagatccactttggaaaggaccagcaaagcttctctggaaaggtgaaggggcagtagtaatacaagataatagtgacataaaagtagtgccaagaagaaaagcaaagatcattagagattatggaaaacagatggcaggtgatgattgtgtggcaagtagacaggatgaggattagaacatggataagtttagtaaaacaccatatgtatatttcaaggaaagcaaaggatggttttatagacatcactatgaaagcactcacccaaaaataagttcagaagtacacatcccactaggggatgctagattggtaataacaacatattggggtctgcatacaggagaaagagattggcatttgggtcatggagtctccgtagaatggaggaaaaagagatatagcacacaagtagaccctgacctagcagaccaactaattcatctgtattactttgattgtttttcagaatctgccataagaaatgccatattaggacatatagttagtcctaggtgtgaatatcaagcaggacataacaaggtaggatctctacagtacctagcactagcagcattaataacaccaaaaaggataaagccacctttgcctagtgttacaaaactaacagaggatagatggaacaagccccagaagaccaagggccacagagggagccatacaatgaatggacatagagcttttagaagaacttaagaatgaagctgttagacattttcctaggatatggctccatggcttagggcaatatatctatgaaacttatggggatacttgggcaggagtggaagccctagtaagaactctgcaacaactgctgtttactcttttagaattgggtgtcgacatagcagaataggcattactcaacgaagaagagcaagaaatggagccagtagatcctagactagagccctggaagcatccaggaagccagcctaaaactgcttgtaccaaatgctattgtaaaaagtgttgcttacattgccaagtttgtttcatgacaaaaggcttaggcatctcctatggcaggaagaagcggagacagcgacgaagagctcctcaagacagtcagactcatcaagcttctctatcaaagcagtaagtagtgcatgtaatgcaacctatacaaatagcagcaatagtagcattagtagtggtaggaataatagcaatagttgtgtggtaaaatattaagacaaagaaaaatagacaggttaattaaaagaataagtaaaagagcagaagacagtggcaatgagagtgaaggagatcaggaagaattatcagcacttgtggagatggggcaccatgctctttgggatattgatgatctatagctagcaagtgaattatataaatataaagtagtaaaaattgaaccattaggagtagcacccaccacggcaaagagaagagtggtgcaaagagaaaaaagagcagtgggaataggagctctgttccttgggttcttgggagcagcaggaagcactatgggcgcagcgtcaatgacgttgacggtacaggccagacaattattgtctggtatagtgcaacagcagaacaatttgctgagggctattgaggcgcaacagcatctgttgcaactcacagtctggggcatcaagcagctccaggcaagagtcctggctgtggaaagatacctaaaggatcaacagctcctggggatttggggttgctctggaaaactcatttgcaccactgctgtgccttggaatgctagttggagtaataaatctctgaatcagatttgggataacatgacttggatgcagtgggaaagagaaattgaaaattacacagacttaatatacaacttaattgaagaatcgcagaaccagcaagaaaagaatgaacaagaattattggaattagataaatgggcaagtttgtggaattggtttacaataacaaactggctgtggtatataaaaatattcataatgatagtaggaggcttgataggtttaagaatagtttttactgtactttctatagtgaatagagttaggcagggatactcaccattgtcgtttcagacccacctcccaaccccgaggggacccgacaggcccgaaggaatcgaagaagaaggtggagagagagacagagacagatccggtcgattagtgaacggattcttagcacttttctgggacgatctgcggagcctgtgcctcttcagctaccaccgcttgagagacttaatcttggttgtaacgaggattgtggaacttctgggacgcagggggtgggaagccctcaagtattggtggagtctcctacagtattggagccaggaactaaagaatagtgctgttaacttgcttaatgtcacagccatagcagtagctgagggaacagatagggttatagaagtagtacaaagaacttatagagctattctccacatacctagaagaataagacagggcttggaaaggcttttgctataagatgggtggcaagtggtcaaaacgtatggagggtggatggcatgctgtaagggaaagaatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcatt

SEQ ID NO: 39 - DNA, 70 bp, Artificial Sequence
  Description of Artificial Sequence  note = Synthetic Construct
dggaagatctgaattcaccatggatcccaaaaagaaaagaaaggtagcatccaatttactaaccgtacac

SEQ ID NO: 40 - DNA, 37 bp, Artificial Sequence
  Description of Artificial Sequence  note = Synthetic Construct
datgccgctcgagctaatcgccatcttccagcaggcg

SEQ ID NO: 41 - DNA, 44 bp, Artificial Sequence
  Description of Artificial Sequence  note = Synthetic Construct
datcgcggatccctgcctcttctcaccaagatgaactcactggt

SEQ ID NO: 42 - DNA, 31 bp, Artificial Sequence
  Description of Artificial Sequence  note = Synthetic Construct
dtttctcgagtcactgccccgcacctgcgcc

SEQ ID NO: 43 - DNA, 7956 bp, Artificial Sequence
  Description of Artificial Sequence  note = Synthetic Construct
dttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggaagctttagacaagatagaggaagagcaaaacaaaagtaagaccaccgcacagcaggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttggaattgcgcgttacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttctcgagtcactgccccgcacctgcgccccagccccgcccagcgcttctgccgtggttccctggtgccgcctcccgcttgccgaagcgcaggccgaaggagttccagttgtagttcggcaggtccttctcccgctgcaccagcaccgcgccctggggtgcggggatctggcggctgtggggggcggacaggcccggctgctggcggctcccggagctctcggggggcggggacagcgaggtccccttgtcgtcatcgtctttgtagtcccgacggctcagcctggcagtagcagctggcttcctctcggtgcacggcaggctctgctccccgggggccaggaggcccagggattctagctgctggcctgtgggtctagaattccccacagaggccaccttttctaatggctccccaaagtgggtggcacagaggaaaagcagtagctgccaagaaaccagtgagttcatcttggtgagaagaggcagggatccatctctatcactgatagggagatctctatcactgatagggagagctctgcttatatagacctcccaccgtacacgcctaccgcccatttgcgtcaatggggcggagttgttacgacattttggaaagtcccgttgattttggttccaaaacaaactcccattgacgtcaatggggtggagacttggaaatccccgtgagtcaaaccgctatccacgcccattgatgtactgccaaaaccgcatcaccatggtaatagcgatgactaatacaattctaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacagtagcttccaccatggccagccgcctggacaagtccaaggtcatcaattccgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattgtacgccctgtccgccgtcggccacttcaccctgggctgtgtgctggaggaccaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcggcggaggcggaggcagtccgcgcgccgatcccaaaaagaaaagaaaggtagcagccatggcctaactcgagtttccctctagcgggatcaattccgccccccccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgacgtcgacgtcaccgccgacgtcgaggtgcccgaaggaccgcgcacctggtgcatgacccgcaagcccggtgcctgacgcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggtacctttaagaccaatgacttacaaggcagctgtagatcttagccactttttaaaagaaaaggggggactggaagggctaattcactcccaacgaagacaagatctgctttttgcttgtacggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 44 - DNA, 6290 bp, Artificial Sequence
  Description of Artificial Sequence  note = Synthetic Construct
dgggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttggaattggaattcaagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcaggtgtcgtgaggatccaccatggccagccgcctggacaagtccaaggtcatcaatggcgccctggagctgctgaacggcgtcggaatcgaaggtttaacaacccgtaaactcgcccagaagctaggtgtagagcagcctacattgtattggcatgtaaaaaataagcgggctttgctcgacgccttacccatcgagatgctggaccgccaccacacccacttctgccccctggagggcgagagctggcaggacttcttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattgtacgccctgtccgccgtcggccacttcaccctgggctgtgtgctggaggagcaggagcatcaagtcgctaaagaagaaagggaaacacctactactgatagtatgccgccattattacgacaagctatcgaattatttgatcgccaaggcgccgagcccgccttcctgttcggcctggagctgatcatctgcggcctggagaagcagctgaagtgcgagagcggcagcgcctacagccgcggcggaggcggaggcagtccgcgcgccgatcccaaaaagaaaagaaaggtagcacgcgtcggcggaggcggaagtgggtccccggccgacgccctggacgacttcgacctggacatgctgccggccgacgccctggacgacttcgacctggacatgctgccggccgacgccctggacgacttcgacctggacatgctgccggccgacgccctggacgacttcgacctggacatgctgccggggtaactaagtaaggatctcgagtttccctctagcgggatcaattccgccccccccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagtcgacgtcaccgccgacgtcgaggtgcccgaaggaccgcgcacctggtgcatgacccgcaagcccggtgcctgacgcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagt

SEQ ID NO: 45 - DNA, 4891 bp, Artificial Sequence
  Description of Artificial Sequence  note = Synthetic Construct
dgggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttggaatttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgaattcggatccacgcgtactagtctcgagcgagtttccctctagcgggatcaattccgccccccccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggtgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgcttaagtcgacgtcaccgccgacgtcgaggtgcccgaaggaccgcgcacctggtgcatgacccgcaagcccggtgcctgacgcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagt

SEQ ID NO: 46 - DNA, 6031 bp, Artificial Sequence
  Description of Artificial Sequence  note = Synthetic Construct
dgggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttggaatttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcggatccaccatggaaactccaaacaccacagaggactatgacacgaccacagagtttgactatggggatgcaactccgtgccagaaggtgaacgagagggcctttggggcccaactgctgccccctctgtactccttggtatttgtcattggcctggttggaaacatcctggtggtcctggtccttgtgcaatacaagaggctaaaaaacatgaccagcatctacctcctgaacctggccatttctgacctgctcttcctgttcacgcttcccttctggatcgactacaagttgaaggatgactgggtttttggtgatgccatgtgtaagatcctctctgggttttattacacaggcttgtacagcgagatctttttcatcatcctgctgacgattgacaggtacctggccatcgtccacgccgtgtttgccttgcgggcacggaccgtcacttttggtgtcatcaccagcatcatcatttgggccctggccatcttggcttccatgccaggcttatacttttccaagacccaatgggaattcactcaccacacctgcagccttcactttcctcacgaaagcctacgagagtggaagctgtttcaggctctgaaactgaacctctttgggctggtattgcctttgttggtcatgatcatctgctacacagggattataaagattctgctaagacgaccaaatgagaagaaatccaaagctgtccgtttgatttttgtcatcatgatcatcttttttctcttttggaccccctacaatttgactatacttatttctgttttccaagacttcctgttcacccatgagtgtgagcagagcagacatttggacctggctgtgcaagtgacggaggtgatcgcctacacgcactgctgtgtcaacccagtgatctacgccttcgttggtgagaggttccggaagtacctgcggcagttgttccacaggcgtgtggctgtgcacctggttaaatggctccccttcctctccgtggacaggctggagagggtcagctccacatctccctccacaggggagcatgaactctctgctgggttcgaaaacctgtattttcagggcgctcgaggagattacaaagatgacgacgataagcgcaacggccatcatcaccatcaccatcaccaccatcactaacgagtttccctctagcgggatcaattccgccccccccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggtgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgcttaagtcgacgtcaccgccgacgtcgaggtgcccgaaggaccgcgcacctggtgcatgacccgcaagcccggtgcctgacgcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagt

SEQ ID NO: 47 - DNA, 9372 bp, Artificial Sequence
  Description of Artificial Sequence  note = Synthetic Construct
dgggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttggaatttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgaattcatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgaatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagctcgaggagattacaaagatgacgacgataagcgcaacggccatcatcaccatcaccattaacgagtttccctctagcgggatcaattccgccccccccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggtgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgcttaagtcgacgtcaccgccgacgtcgaggtgcccgaaggaccgcgcacctggtgcatgacccgcaagcccggtgcctgacgcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagt

SEQ ID NO: 48 - DNA, 9384 bp, Artificial Sequence
  Description of Artificial Sequence  note = Synthetic Construct
dgggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttggaatttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgaattcatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgaatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagctcgaggagattacaaagatgacgacgataagcgcaacggccatcatcaccatcaccatcaccaccatcactaacgagtttccctctagcgggatcaattccgccccccccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggtgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgcttaagtcgacgtcaccgccgacgtcgaggtgcccgaaggaccgcgcacctggtgcatgacccgcaagcccggtgcctgacgcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagt

SEQ ID NO: 49 - DNA, 5015 bp, Artificial Sequence
  Description of Artificial Sequence  note = Synthetic Construct
dagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaaggatccaccatgggcaatgcctccaatgactcccagtctgaggactgcgagacgcgacagtggcttcccccaggcgaaagcccagccatcagctccgtcatgttctcggccggggtgctggggaacctcatagcactggcgctgctggcgcgccgctggcggggggacgtggggtgcagcgccggccgcaggagctccctctccttgttccacgtgctggtgaccgagctggtgttcaccgacctgctcgggacctgcctcatcagcccagtggtactggcttcgtacgcgcggaaccagaccctggtggcactggcgcccgagagccgcgcgtgcacctacttcgctttcgccatgaccttcttcagcctggccacgatgctcatgctcttcgccatggccctggagcgctacctctcgatcgggcacccctacttctaccagcgccgcgtctcgcgctccgggggcctggccgtgctgcctgtcatctatgcagtctccctgctcttctgctcgctgccgctgctggactatgggcagtacgtccagtactgccccgggacctggtgcttcatccggcacgggcggaccgcttacctgcagctgtacgccaccctgctgctgcttctcattgtctcggtgctcgcctgcaacttcagtgtcattctcaacctcatccgcatgcaccgccgaagccggagaagccgctgcggaccttccctgggcagtggccggggcggccccggggcccgcaggagaggggaaagggtgtccatggcggaggagacggaccacctcattctcctggctatcatgaccatcaccttcgccgtctgctccttgcctttcacgatttttgcatatatgaatgaaacctcttcccgaaaggaaaaatgggacctccaagctcttaggtttttatcaattaattcaataattgacccttgggtctttgccatccttaggcctcctgttctgagactaatgcgttcagtcctctgttgtcggatttcattaagaacacaagatgcaacacaaacttcctgttctacacagtcagatgccagtaaacaggctgaccttgaaaacctgtattttcagggcgctcgaggagattacaaaaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagg

SEQ ID NO: 50 - DNA, 6040 bp, Artificial Sequence
  Description of Artificial Sequence  note = Synthetic Construct
dgggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttggaatttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcggatccaccatgggcaatgcctccaatgactcccagtctgaggactgcgagacgcgacagtggcttcccccaggcgaaagcccagccatcagctccgtcatgttctcggccggggtgctggggaacctcatagcactggcgctgctggcgcgccgctggcggggggacgtggggtgcagcgccggccgcaggagctccctctccttgttccacgtgctggtgaccgagctggtgttcaccgacctgctcgggacctgcctcatcagcccagtggtactggcttcgtacgcgcggaaccagaccctggtggcactggcgcccgagagccgcgcgtgcacctacttcgctttcgccatgaccttcttcagcctggccacgatgctcatgctcttcgccatggccctggagcgctacctctcgatcgggcacccctacttctaccagcgccgcgtctcgcgctccgggggcctggccgtgctgcctgtcatctatgcagtctccctgctcttctgctcgctgccgctgctggactatgggcagtacgtccagtactgccccgggacctggtgcttcatccggcacgggcggaccgcttacctgcagctgtacgccaccctgctgctgcttctcattgtctcggtgctcgcctgcaacttcagtgtcattctcaacctcatccgcatgcaccgccgaagccggagaagccgctgcggaccttccctgggcagtggccggggcggccccggggcccgcaggagaggggaaagggtgtccatggcggaggagacggaccacctcattctcctggctatcatgaccatcaccttcgccgtctgctccttgcctttcacgatttttgcatatatgaatgaaacctcttcccgaaaggaaaaatgggacctccaagctcttaggtttttatcaattaattcaataattgacccttgggtctttgccatccttaggcctcctgttctgagactaatgcgttcagtcctctgttgtcggatttcattaagaacacaagatgcaacacaaacttcctgttctacacagtcagatgccagtaaacaggctgaccttgaaaacctgtattttcagggcgctcgaggagattacaaagatgacgacgataagcgcaacggccatcatcaccatcaccatcaccaccatcactaacgagtttccctctagcgggatcaattccgccccccccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggtgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgcttaagtcgacgtcaccgccgacgtcgaggtgcccgaaggaccgcgcacctggtgcatgacccgcaagcccggtgcctgacgcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagt

SEQ ID NO: 51 - DNA, 7647 bp, Artificial Sequence
  Description of Artificial Sequence  note = Synthetic Construct
dgggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttggaattaattgcgcgttacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttctcgagatctgagtccggacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctaggatccatctctatcactgatagggagatctctatcactgatagggagactctgcttatatagacctcccaccgtacacgcctaccgcccatttgcgtcaatggggcggagttgttacgacattttggaaagtcccgttgattttggttccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaggaattcaagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcaggtgtcgtgaccatggccagccgcctggacaagtccaaggtcatcaattccgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattgtacgccctgtccgccgtcggccacttcaccctgggctgtgtgctggaggaccaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcggcgccatggcctaactcgagtttccctctagcgggatcaattccgccccccccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagtcgacgtcaccgccgacgtcgaggtgcccgaaggaccgcgcacctggtgcatgacccgcaagcccggtgcctgacgcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagt

SEQ ID NO: 52 - DNA, 4987 bp, Artificial Sequence
  Description of Artificial Sequence  note = Synthetic Construct
dgggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttggaatttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgaattcggatccacgcgtactagtctcgaggaaaacctgtattttcagggcgctcgaggagattacaaagatgacgacgataagcgcaacggccatcatcaccatcaccatcaccaccatcactaacgagtttccctctagcgggatcaattccgccccccccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggtgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgcttaagtcgacgtcaccgccgacgtcgaggtgcccgaaggaccgcgcacctggtgcatgacccgcaagcccggtgcctgacgcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagt

(19) United States
(12) Patent Application Publication
(10) Pub. No.: US 2009/0210952 A1
(43) Pub. Date: Aug. 20, 2009

(54) Compositions and Methods Related to Controlled Gene Expression Using Viral Vectors

(76) Inventors: Xiaoyun Wu, Birmingham, AL (US); John Kappes, Homewood, AL (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/096,651

(22) PCT Filed: Dec. 18, 2006
(86) PCT No.: PCT/US2006/048243

§ 371 Date: Dec. 17, 2008

Related U.S. Application Data
(60) Provisional application No. 60/751,407, filed on Dec. 16, 2005.
(60) Provisional application No. 60/751,117, filed on Dec. 16, 2005.

(51) Int. Cl. A01K 67/027; A01K 67/033

(52) U.S. Cl. 800/13; 800/22

(57) ABSTRACT

Provided herein are methods and compositions related to viral vectors. Also provided herein are methods and compositions for the efficient transfection of a host, for example through the highly efficient lentivector delivery system, and for the exquisite control of the timing and level of expression of the transferred sequence of interest by the simple administration of a modulator to the host harboring the transferred sequence of interest. Also disclosed are methods of making transgenic mice and transgenic mice made using compositions and methods relating to viral vectors.

### CROSS-REFERENCE TO RELATED APPLICATIONS

This application claims benefit of U.S. Provisional Application No. 60/751,407, filed Dec. 16, 2005 and U.S. Provisional Application No. 60/751,117 also filed Dec. 16, 2005. U.S. Provisional Application No. 60/751,407, filed Dec. 16, 2005 and U.S. Provisional Application No. 60/751,117 also filed Dec. 16, 2005, are hereby incorporated herein by reference in their entirety.

### ACKNOWLEDGEMENTS

This invention was made with government support under grants R01 A147717 and R01 AI48852 from the National Institutes of Health and National Institute of Allergy and Infectious Diseases. The government has certain rights in the invention.

### BACKGROUND

It is frequently desirable to transfer and control the expression of a sequence of interest in cells or living organisms, whether the subject is cells in culture, or a living organism such as an animal model or human subject in need of receiving a therapeutic gene. When lentiviral vectors based on HIV are used as the mode of transferring and/or expressing sequences of interest, concerns arise regarding the safety of their use, since the virus is the etiological agent for AIDS. Further concerns involve the possibility of insertional activation of cellular oncogenes, the ability of the vector or construct to successfully and effectively associate with ribosomes, and the ability of the vector or construct to successfully signal for nuclear importation. To date, there has not been created a lentiviral vector that is safe and effective for use in transferring and/or expressing sequences of interest in mammalian hosts or cells, and which provides the important ability to both induce and reverse expression of the transferred genes or sequences of interest.

Lentiviruses are complex retroviruses which, based on their higher level of complexity, can integrate into the genome of nonproliferating cells and modulate their life cycles, as in the course of latent infection. These viruses include HIV-1, HIV-2 and SIV. Like other retroviruses, lentiviruses possess gag, pol and env genes which are flanked by two long terminal repeat (LTR) sequences. Each of these genes encodes multiple proteins, initially expressed as one precursor polyprotein. The gag gene encodes the internal structural (matrix capsid and nucleocapsid) proteins. The pol gene encodes the RNA-directed DNA polymerase (reverse transcriptase, integrase and protease). The env gene encodes viral envelope glycoproteins and additionally contains a cis-acting element (RRE) responsible for nuclear export of viral RNA. The 5′ and 3′ LTRs serve to promote transcription and polyadenylation of the virion RNAs. The LTR contains all other cis-acting sequences necessary for viral replication. Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the result is a cis defect which prevents encapsidation of genomic RNA. However, the resulting mutant is still capable of directing the synthesis of all virion proteins. A comprehensive review of lentiviruses, such as HIV, is provided, for example, in Field's Virology (Raven Publishers), eds. B. N. Fields et al., (1996).

Although lentiviral vectors are very useful for a variety of applications, the possibility of generating replication-competent retrovirus (RCR) through genetic recombination raises concerns for safety. One way investigators have, attempted to overcome such a problem is to construct an HIV-based packaging system (trans-lentiviral) that splits gag/gag-pol into two parts: one that expresses gag/gag-pro and another that expresses reverse transcriptase and integrase as fusion partners of viral protein R (Vpr). However, such a method was found to have drawbacks, as the efficiency of producing infectious viral vector particles was far less than ideal.

Additional methods and systems for producing efficient retroviral packaging cell lines, particularly lentiviral packaging cell lines, which do not generate recombinant retrovirus would be of a great value.

### SUMMARY OF THE INVENTION

Provided herein is a solution to the problems enumerated above, by combining a gene transfer construct or other expression system and a gene regulation system for the efficient delivery and controlled expression of genes into cells and living organisms. The present invention therefore provides for the efficient transfection of the host, for example through the highly efficient lentivector delivery system, and for the exquisite control of the timing and level of expression of the transferred sequence of interest by the simple administration of a modulator (e.g., an antibiotic such as tertracycline) to the host harboring the transferred sequence of interest. The present invention offers the additional benefit of achieving this efficient transfection and regulation in non-dividing cells in hosts of several species, such as rodents, primates, and canines.

Provided herein are gene transfer constructs and expression systems. The gene transfer constructs and expression systems of the present invention can be lentiviral vectors. These constructs comprise various components that make them both safe and effective for transferring sequences of interest to mammalian host cells, and further provide the extremely important ability to exercise great control over the expression of the transferred sequences of interest in the mammalian host cells by administration of a suitable modulator to cells or subjects containing the inducible and reversible gene transfer constructs. The gene transfer constructs of the present invention can comprise one or more of the following: a self-inactivating 5′ LTR, a regulator-responsive promoter, a nuclear import signal, a promoter operatively associated with a nucleic acid encoding a regulator-responsive receptor, an RNA stabilizing element, or a self-inactivating 3′ LTR. The disclosed gene transfer constructs are useful for packaging and delivering DNA to both dividing and non-dividing cells. The packaging and transfer constructs disclosed herein can be used in combination with each other and also used in combination with the other packaging and gene transfer constructs, systems, and methods known in the art as well as the systems and methods disclosed herein.

Also provided herein are specific gene transfer constructs and methods for using the constructs to inducibly and reversibly express sequences of interest in target cells. Further provided are ex vivo methods employing the disclosed gene transfer constructs as expression systems for treating mammalian subjects. Also provided are methods of making an animal model of expression of a sequence of interest. Furthermore, the present invention provides cells incorporating or containing the gene transfer constructs or expression systems disclosed herein. The disclosed gene transfer constructs thus facilitate the construction of stable, inducible/reversible cell lines, as the pseudotype lentivectors can transduce many cell types that are refractory to standard DNA transfection techniques.

Also provided are bidirectional promoters that can drive expression of at least two separate sequences in opposite directions. The disclosed bidirectional promoters can also be used with the packaging and gene transfer constructs disclosed herein.

Also provided are cell lines comprising the various gene transfer constructs described herein.

Also disclosed herein are gene transfer constructs wherein the construct is capable of generating non-replication competent recombinants.

Also provided are expression systems comprising the various gene transfer constructs described herein. Also provided are cell lines comprising the gene transfer constructs or expression systems described herein and cells made by the methods described herein.

Also provided are methods of selectively regulating the expression of a gene of interest comprising introducing the gene transfer constructs disclosed herein to a target cell.

Also provided are methods of making a recombinant protein, antibodies, and transgenic animals.

Also provided herein are packaging constructs comprising nucleic acid sequences encoding Gag and Gag-Pro-Pol polyproteins. These constructs are safe, but provide improved packaging efficiency as compared to constructs available prior to this invention. Also provided herein are packaging constructs comprising nucleic acid sequences encoding Gag and Gag-Pro-Pol polyproteins that further comprise one or more mutations in the nucleic acid sequences encoding Gag and Gag-Pro-Pol polyproteins that reduce frame-shifting or translational read-through required for the synthesis of Gag-Pro and Gag-Pro-Pol polyproteins.

Also provided is a packaging construct comprising a first and a second nucleic acid sequence, wherein the first nucleic acid sequence encodes a Gag polyprotein, wherein the second nucleic acid sequence encodes a Gag-Pro polyprotein, wherein the first and a second nucleic acid sequences comprise one or more mutations that reduce frame-shifting or translational read-through, wherein the first and second nucleic acid sequences are expressed from different coding regions of the same nucleotide sequence, and wherein the first and second nucleic acid sequences are operably linked to at least one transcriptional control element.

Also provided herein are packaging constructs comprising a first, second and a third nucleic acid sequence, wherein the first nucleic acid sequence encodes a Gag polyprotein, wherein the second nucleic acid sequence encodes a Gag-Pro polyprotein, and wherein the third nucleic acid sequence encodes a Vpr-Reverse Transcriptase-Integrase protein.

Also provided herein are packaging constructs wherein Gag and Gag-Pol are in trans, wherein the nucleic acid sequence that encodes a Gag polyprotein and the nucleic acid sequence that encodes a Gag-Pro polyprotein comprise one or more mutations that reduce frame-shifting or translational read-through, and the nucleic acid sequence that encodes a Gag polyprotein and the nucleic acid sequence that encodes a Gag-Pro polyprotein are operably linked to at least one transcriptional control element.

Also provided are cell lines, packaging systems, and expression systems comprising the various packaging constructs described herein. Also provided are cell lines comprising the expression systems described herein.

Optionally, the packaging constructs described herein are capable of generating non-replication competent recombinants.

Also provided are methods of making a virus-like particle.

Further provided herein are methods of making and using the cell lines, packaging constructs, gene transfer constructs, packaging systems and expression systems described herein.

Also provided herein are methods of screening for an agent that modulates viral particle formation.

Further provided are vaccines comprising the gene transfer constructs disclosed herein and methods of inducing an immune response in a subject comprising administering to a subject the vaccines disclosed herein.

The present invention therefore successfully combines an efficient sequence of interest delivery system with a tightly regulated sequence of interest expression system, and represents a significant advance in sequence of interest delivery and expression technology.

### BRIEF DESCRIPTION OF THE DRAWINGS

The accompanying drawings, which are incorporated in and constitute a part of this specification, illustrate several aspects described below.

FIG. 1 shows a comparison between a mutated gag sequence required for frame-shifting comprising point mutations and to a wild-type gag sequence.

FIG. 2 shows a comparison between a mutated gag-pol sequence required for frame-shifting comprising point mutations and to a wild-type gag-pol sequence.

FIG. 3 shows the loop structure in HIV gag and HIV gag-pol required for frame-shifting.

FIG. 4 shows an altered sequence of loop structure in HIV gag and HIV gag-pol required for frame-shifting that results in the disruption of the loop structure required for frame-shifting.

FIG. 5 shows the results of FACS analysis of GFP expression in the blood cells of transgenic CAG-founders before and after 18 days of feeding the mice DOX.

FIG. 6 shows the induction kinetics of GFP expression in the blood cell of transgenic CAG-founders.

FIG. 7 shows that both the human and mouse H1 promoters are capable of expressing shRNA designed to target eGFP, which in turn can efficiently silence eGFP expression in HeLa cells.

FIG. 8 shows that both the human and mouse H1 promoters are capable of expressing shRNA designed to target eGFP, which in turn can efficiently silence eGFP expression in human T cells

FIG. 9 shows that a single, inducible lentivector comprising shRNA that targets mouse CXCR4 could inducibly reduce the expression of mouse endogenous CXCR4 protein.

FIG. 10 shows that the multiple copies of the integrated a single, inducible lentivector comprising shRNA that targets mouse CXCR4 can elicit a high level of the gene silencing.

FIG. 11 shows induction of siRNA expression to reduce GFP in blood cell of transgenic mice by DOX.

FIG. 12 shows induction of siRNA expression to reduce GFP in blood cell of transgenic mice by DOX.

FIG. 13 shows the expression level of GFP in blood cells of a non transgenic mouse and transgenic CAG-founders F1-6# and F1-9# before the mice were fed DOX.

FIG. 14 shows that the expression level of GFP in the in blood cells of a non transgenic mouse and, transgenic CAG-founders F1-4# and F1-11# at 10, 17, 27 days after the mice were fed DOX.

FIG. 15 shows examples of gene transfer constructs as disclosed herein.

FIG. 16 shows A) an HIV-based lentiviaral vector comprising hCCR1-m that can be used to generate a cell line that can inducibly and reversibly express the human CCR1 gene. B) shows the C-terminal amino acids sequence of CCR1-m. The stop codon of CCR1 is mutated and replaced for TEV protease site (ENLYFQG). The M2 flag is inserted between TEV and 10 Histine amino acids in order to analyze the protein (CCR1-m) expression and Purification. The 10 His-tag serve as the purification of CCR1-m using Ni-NTA columns.

FIG. 17 shows A) an HIV-based lentiviaral vector comprising hEP2R that can be used to generate a cell line that can inducibly and reversibly express the human EP2 gene. B) shows the C-terminal amino acids sequence of hEP2R-m. The stop codon of is mutated and replaced for TEV protease site (ENLYFQG). The M2 flag is inserted between TEV and 10 Histine amino acids in order to analyze the protein (hEP2R-m) expression and purification. The 10 His-tag serve as the purification of hEP2R-m using Ni-NTA columns.

### DETAILED DESCRIPTION

Before the present compounds, compositions, articles, devices, and/or methods are disclosed and described, it is to be understood that the aspects described below are not limited to specific synthetic methods or specific administration methods, as such may, of course, vary. It is also to be understood that the terminology used herein is for the purpose of describing particular aspects only and is not intended to be limiting.

It must be noted that, as used in the specification and the appended claims, the singular forms “a,” “an” and “the” include plural referents unless the context clearly dictates otherwise.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In one aspect, the subject is a mammal such as a primate or a human.

“Optional” or “optionally” means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

The phrase “packaging cell line” or “packaging cells” refers to cells (typically a mammalian cell line) that contain the necessary coding sequences to produce viral particles or viral-like particles, which are defective in the ability to package viral RNA and produce replication-competent helper-virus. When the packaging function is provided within the cells, the packaging cell line or packaging cells produce recombinant retrovirus, thereby becoming a “retroviral producer cell line” or “retroviral producer cells”.

The term “retrovirus” refers to any known retrovirus (e.g., type c retroviruses, such as Moloney murine leukemia virus (MoMuLV), Harvey murine sarcoma virus (HaMuSV), murine mammary tumor virus (MuMTV), gibbon ape leukemia virus (GaLV), feline leukemia virus (FLV) and Rous Sarcoma Virus (RSV)). “Retroviruses” of the invention also include human T cell leukemia viruses, HTLV-1 and HTLV-2, and the lentiviral family of retroviruses, such as human Immunodeficiency viruses, HIV-1, HIV-2, simian immunodeficiency virus (SIV), feline immunodeficiency virus (FIV), equine immunodeficiency virus (EIV), and other classes of retroviruses.

The terms “Gag polyprotein” or “Gag protein”, “Pro polyprotein” or “Pro protein”, and “Pol polyprotein” or “Pol protein” refer to the multiple proteins encoded by retroviral gag, pro, and pol genes which are typically expressed as a single precursor “polyprotein”. For example, HIV gag encodes, among other proteins, p17, p24, p7 and p6. HIV pro encodes viral protease: HIV pol encodes, among other proteins, protease (PR), reverse transcriptase (RT) and integrase (IN). As used herein, the term “polyprotein” shall include all or any portion of gag, pro, or pol polyproteins.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “sequence of interest” or “gene of interest” can mean a nucleic acid sequence (e.g., a therapeutic gene), that is partly or entirely heterologous, i.e., foreign, to a cell into which it is introduced.

The term “sequence of interest” or “gene of interest” can also mean a nucleic acid sequence, that is partly or entirely homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). For example, a sequence of interest can be cDNA, DNA, or mRNA.

The term “sequence of interest” or “gene of interest” can also mean a nucleic acid sequence, that is partly or entirely complementary to an endogenous gene of the cell into which it is introduced. For example, the sequence of interest can be micro RNA, shRNA, or siRNA.

A “sequence of interest” or “gene of interest” can also include one or more transcriptional regulatory sequences and any other nucleic acid, such as introns, that may be necessary for optimal expression of a selected nucleic acid. A “protein of interest” means a peptide or polypeptide sequence (e.g., a therapeutic protein), that is expressed from a sequence of interest or gene of interest.

A “gene transfer construct” refers to a nucleic acid sequence that is typically used in conjunction with other lentiviral or trans-lentiviral vector system vectors to produce viral particles, e.g., so that the viral particles can then transduce a target cell of interest.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “transformation” and “transfection” mean the introduction of a nucleic acid, e.g., an expression vector, into a recipient cell including introduction of a nucleic acid to the chromosomal DNA of said cell.

The term “RNA export element” refers to a cis-acting post-transcriptional regulatory element that regulates the transport of an RNA transcript from the nucleus to the cytoplasm of a cell. Examples of RNA export elements include, but are not limited to, the human immunodeficiency virus (HIV) rev response element (RRE) (see e.g., Cullen et al. (1991) J. Virol. 65: 1053; and Cullen et al. (1991) Cell 58: 423-426), and the hepatitis B virus post-transcriptional regulatory element (PRE) (see e.g., Huang et al. (1995) Molec. and Cell. Biol. 15(7): 3864-3869; Huang et al. (1994) J. Virol. 68(5): 3193-3199; Huang et al. (1993) Molec. and Cell. Biol 13(12): 7476-7486), and U.S. Pat. No. 5,744,326, which are all hereby incorporated by reference in their entirety regarding RNA export elements). Generally, the RNA export element is placed within the 3′ UTR of a gene and can be inserted as one or multiple copies. RNA export elements can be inserted into any or all of the separate vectors generating the packaging cell lines of the present invention.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15.

A “virus-like particle” or “viral particle” refers to a proteinaceous, capsid-like virion that is produced by expression of at least one of the following viral genes, gag, pro, rt, in, and env, in a host cell. The particle produced preferably further contains an mRNA equivalent of the gene transfer vector and is infectious, or can be made infectious, for a given cell type to be transduced.

### Compositions

The gag and pol genes of human immunodeficiency virus type 1 (HIV-1) are initially expressed as the precursor polyproteins Gag and Gag-Pro-Pol. During or after budding, these precursors are processed by the viral protease (PR) into their mature products. The 55-kDa Gag precursor generates matrix (MA), capsid (CA), spacer peptide p2, nucleocapsid (NC), spacer peptide p1, and p6. The 160-kDa Gag-Pro-Pol polyprotein generates MA, CA, p2, NC, p6, PR, reverse transcriptase (RT), and integrase (IN). The Gag and Gag-Pro-Pol polyproteins are encoded by the same mRNA but are not synthesized at the same rate. An infrequent ribosomal frameshifting event generates an approximate 20:1 ratio of Gag to Gag-Pro-Pol production. The maintenance of this ratio is critical for viral particle formation and infectivity.

Intracellular expression of Gag alone is sufficient to produce viral-like particles (VLPs). Moreover, there is an important role for Gag and viral genomic RNA interactions in the assembly process, with the packaging and dimerization of the genomic RNA primarily occurring via RNA-Gag interactions. The NC domain of Gag binds to viral RNA and has been shown to facilitate both the RNA packaging and the dimerization processes. The initial interaction between genomic RNA and HIV-1 Gag appears to occur via the NC sequences within the Gag precursor, as HIV-1 with defective viral PR still packages RNA. Furthermore, analysis of wild-type (WT) and PR-defective (PR-) virions has revealed that dimerization of the genomic RNA in HIV-1 initiates prior to proteolytic processing, showing that Gag and Gag-Pro-Pol precursor proteins can support RNA dimerization independently of protein processing.

In addition to gag, pol, and env, lentiviruses, unlike other retroviruses, have several “accessory” genes with regulatory or structural function. Specifically, HIV-1 possesses at least six such genes, including Vif, Vpr, Tat, Rev, Vpu and Nef. The closely related HIV-2 does not code for Vpu, but codes for another unrelated protein, Vpx, not found in HIV-1.

The HIV-1 Vpr gene encodes a 14 kD protein (96 amino acids) (Myers et al. (1993) Human Retroviruses and AIDS, Los Alamos National Laboratory, N.M.). The Vpr open reading frame is also present in most HIV-2 and SIV viruses. Amino acid comparison between HIV-2 Vpr and Vpx shows regions of high homology suggesting that Vpx may have arisen by duplication of the Vpr gene. Vpr and Vpx are present in mature viral particles in multiple copies, and have been shown to bind to the p6 protein which is part of the gag-encoded precursor polyprotein involved in viral assembly (WO 96/07741; WO 96/32494). Thus, incorporation of Vpr and Vpx into viral particles occurs by way of interaction with p6 (Lavallee et al. (1994) J. Virol. 68: 1926-1934; and Wu et al. (1994) J. Virol. 68:6161). It has been further shown that Vpr associates, in particular, with the carboxy-terminal region of p6. It has been shown that Vpr and Vpx, expressed in trans with respect to the HIV genome, can be used to target heterologous proteins to HIV virus (WO 96/07741; WO 96/32494). A description of the structure and function of Vpr and Vpx, including the full-length nucleotide and amino acid sequences of these proteins and their binding domains are also provided in WO 96/07741, as well as in Zhao et al. (1994) J Biol Chem. 269(22):1577 (Vpr); Mahalingham et al. 91995) Virology 207:297 (Vpr); and Hu et al. (1989) Virology 173:624) (Vpx). Other relevant references relating to Vpr include, for example, Kondo et al. (1995) J. Virol 69:2759; Lavallee et al. (1994) J. Virol. 68:1926; and Levy et al. (1993) Cell 72:541. Other relevant references relating to Vpx include, for example, Wu et al. (1994) J. Virol. 68:6161. These references are incorporated herein by reference in their entirety for their teachings of the structure and functions of Vpr and Vpx

The retroviral integrase (IN) protein catalyzes integration of the provirus and is essential for persistence of the infected state in vivo. Significant progress has been made in the understanding of this critical enzyme, especially its protein structure and the biochemical mechanism of the catalytic integration reaction (Brown, P. 1997. Integration, p. 161-204. In J. M. Coffin, S. H. Hughes, and H. E. Varmus (ed.), Retroviruses. Cold Spring Harbor Laboratory, Cold Spring Harbor, N.Y.; Dyda, F., A. B. Hickman, T. M. Jenkins, A. Engelman, R. Craigie, and D. R. Davies. 1994. Crystal structure of the catalytic domain of HIV-1 integrase: similarity to other polynucleotidyl transferases. Science 266:1981-1986; Katz, R. A., and A. M. Skalka. 1994. The retroviral enzymes. Annu. Rev. Biochem. 63:133-173. All of these references are incorporated herein by reference in their entirety for their teaching of IN's protein structure and the biochemical mechanism of the catalytic integration reaction). HIV-1 IN is expressed and assembled into the virus particle as a part of a larger, 160-kDa Gag-Pol precursor polyprotein (Pr160^{Gag-Pol}) that contains other Gag (matrix, capsid, nucleocapsid, and p6) and Pol (protease, reverse transcriptase [RT], and IN) components. After assembly, Pr160^{Gag-Pol} is proteolytically processed by the viral protease to liberate the individual Gag and Pol components, including the 32-kDa IN protein. Studies on IN function using replicating virus have suggested that in addition to catalyzing integration of the viral cDNA, IN can have other effects on virus replication (Gallay, P., S. Swingler, J. Song, F. Bushman, and D. Trono. 1995. HIV nuclear import is governed by the phosphotyrosine-mediated binding of matrix to the core domain of integrase. Cell 83:569-576; Leavitt, A. D., G. Robles, N. Alesandro, and H. E. Varmus. 1996. Human immunodeficiency virus type 1 integrase mutants retain in vitro integrase activity yet fail to integrate viral DNA efficiently during infection. J. Virol. 70:721-728; Masuda, T., V. Planelles, P. Krogstad, and I. S. Y. Chen. 1995. Genetic analysis of human immunodeficiency virus type 1 integrase and the U3 att site: unusual phenotype of mutants in the zinc finger-like domain. J. Virol. 69:6687-6696. All of these references are incorporated herein by reference in their entirety for their teaching of IN's protein structure and the biochemical mechanism of the catalytic integration reaction). In studies with proviral clones, it has been shown that IN gene mutations can affect virus replication at multiple levels. Mutations in the IN gene can affect the Gag-Pol precursor protein and alter assembly, maturation, and other subsequent viral events. IN gene mutations can also affect the mature IN protein and its organization within the virus particle and the nucleoprotein preintegration complex. Therefore, such mutations are pleiotropic and can alter virus replication through various mechanisms and at different stages in the virus life cycle.

Reverse transcription is catalyzed by RT, and although reverse transcription can occur in vitro with recombinant RT, template, and primer, the process is more complex in vivo. In the context of a replicating virus, complete synthesis of the viral cDNA is not as simple as putting together different proteins and nucleic acids; rather, it is a complex, multistep process involving a number of transitional structures. Within the infected cell, reverse transcription takes place in the context of a nucleic acid-protein (nucleoprotein) complex that includes other viral and cellular factors. Moreover, synthesis of the viral cDNA is greatly dependent on the proper execution of numerous molecular events that precede reverse transcription.

Disclosed herein are packaging and gene transfer constructs. Protocols for producing recombinant retroviral vectors, and for transforming packaging cell lines, are well known in the art [Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad Sci USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573. All of these references are incorporated herein by reference in their entirety for their teachings of protocols for producing recombinant retroviral constructs and vectors, and for transforming cell lines). Moreover, suitable retroviral sequences which can be used in the present invention can be obtained from commercially available sources. For example, such sequences can be purchased in the form of retroviral plasmids, such as pLJ, pZIP, pWE and pEM. Suitable packaging sequences that can be employed in the vectors of the invention are also commercially available including, for example, plasmids ψCrip, ψCre, ψ2 and ψAm. Thus, while the present invention shall be described with respect to particular embodiments (e.g., particular lentiviral vectors), other retroviral vectors for use in the invention can be prepared in accordance with the guidelines described herein. In addition, the gene transfer vectors disclosed herein can be used with the packaging and expression systems disclosed herein.

Specifically, disclosed are packaging constructs comprising nucleic acid sequences encoding Gag and Gag-Pro-Pol proteins. Optionally, the packaging construct comprises a first and a second nucleic acid sequence, wherein the first nucleic acid sequence encodes a Gag protein, wherein the second nucleic acid sequence encodes a Gag-Pro-Pol protein, wherein the first and a second nucleic acid sequences each comprise one or more mutations that reduce frame-shifting or translational read-through, wherein the first and second nucleic acid sequences are expressed from different coding regions of the same nucleotide sequence, and wherein the first and second nucleic acid sequences are operably linked to at least one transcriptional control element.

Also disclosed is a packaging construct comprising a first and a second nucleic acid sequence, wherein the first nucleic acid sequence encodes a Gag protein, wherein the second nucleic acid sequence encodes a Gag-Pro protein, wherein the first and a second nucleic acid sequences each comprise one or more mutations that reduce frame-shifting or translational read-through, wherein the first and second nucleic acid sequences are expressed from different coding regions of the same nucleotide sequence, and wherein the first and second nucleic acid sequences are operably linked to at least one transcriptional control element. In this construct, the nucleic acid sequence that normally encodes poly RT and IN is removed or mutated, such that the Pol or RT-IN proteins are not expressed. Removing or mutating the nucleic acid sequence that encodes the Pol proteins further decreases the possibility of generating replication-competent retrovirus (RCR) through genetic recombination. RT and IN can then be expressed from a separate construct (in trans). For example, reverse transcriptase and integrase can be expressed as fusion partners of viral protein R (Vpr).

Also disclosed herein are packaging constructs comprising a first, second and a third nucleic acid sequence, wherein the first nucleic acid sequence encodes a Gag protein, wherein the second nucleic acid sequence encodes a Gag-Pro protein, and wherein the third nucleic acid sequence encodes a Vpr-Reverse Transcriptase-Integrase protein. Furthermore, the first and second nucleic acid sequences can comprise one or more mutations that reduce frame-shifting or translational read-through, wherein the first, second and a third nucleic acid sequences are expressed from different coding regions of the same nucleotide sequence, and wherein the first, second and third nucleic acid sequences are operably linked to at least one transcriptional control element. In this construct, the nucleic acid sequence capable of encoding the Pol protein can be removed or mutated, such that the Pol proteins are not expressed. The reverse transcriptase and integrase can be supplied in trans by the nucleic acid sequence that encodes a Vpr-Reverse Transcriptase-Integrase protein.

Also disclosed is an IRES or IRES-like element located further downstream to control Vpr-RT-IN. IRESs and IRES-like elements are described below. For example, disclosed is a packaging construct further comprising an element between the first or second nucleic acid sequence and the third nucleic-acid sequence, wherein the third nucleic acid sequence is not located between the first and second nucleic acid sequences, and wherein the element provides differential expression between the first or second nucleic acid sequences and the third nucleic acid sequence. Examples include an internal ribosomal entry site or an internal ribosomal entry site-like element. IRES and IRES-like elements useful with this method are described herein. The IRES can be, for example, the EMC-virus IRES, HCV-virus IRES, or an IRES of a different origin. Other examples of IRESs that can be used include, but are not limited to the IRES present in the IRES database at http://ifr31w3.toulouse.inserm.fr/IRESdatabase/.

Also disclosed are packaging constructs that further comprise a nucleic acid sequence that comprise a rev response element.

In nature, the Gag and Gag-Pol proteins are encoded by partially-overlapping open reading frames. Gag has its own initiation and termination codons, while the synthesis of the HIV-1 Gag-Pol precursor results from a frameshifting event that occurs at a frequency of approximately 5 to 10% of that of the translation of Gag. Other retroviruses also use similar frameshifting mechanisms or a read-through suppression mechanism to regulate the expression of Gag-Pol or Gag-Pro proteins. Thus, intracellular Gag/Gag-Pol ratios are regulated during the replication of all retroviruses. The HIV frameshift site (a heptanucleotide AU-rich sequence) is found at the 3′ end of the nucleocapsid (NC) coding sequence. This site and a stem structure immediately downstream stall the ribosome during the synthesis of Gag, allowing the ribosome to slip back one nucleotide to enable the infrequent (relative to Gag) synthesis of the Gag-Pol fusion protein.

Multimerization of the Gag protein gives rise to viral particles, while expression of Gag-Pol precursor protein ensures that viral enzymes are incorporated into viral particles during viral assembly. During and after release of virions from cells, the Gag precursor protein is cleaved by viral protease (PR) into mature proteins: matrix, capsid (CA), NC, p6, and two spacer peptides, p2 and p1. Gag-Pol fusion is cleaved to yield matrix, CA, p2, and NC, as well as transframe protein, PR, reverse transcriptase (RT), and integrase (IN).

The synthesis of Gag precursor protein alone has been reported to be sufficient for the assembly and release of virus-like particles. Incorporation of Gag-Pol or its mature products into virions is required for infectivity, as they mediate the synthesis and integration of viral cDNA in infected cells. In addition, cleavage of the precursor proteins by PR is required for morphological maturation of the virion core and generation of infectious viral particles. Viral genomic RNA is also packaged into virions during assembly, driven by the genomic RNA packaging sequence found near the 5′ end of the genome and interaction with the NC domain of Gag.

Like other retroviruses, HIV-1, for example, has a dimeric RNA genome. In vitro dimerization analysis of HIV-1 viral RNA has mapped a 50- to 60-nucleotide sequence, termed the dimer initiation sequence, that is important for the formation of the dimeric RNA complex. Mutations in the dimer initiation sequence hinder genomic RNA dimerization and virion RNA packaging and result in the production of noninfectious viral particles. It is thought that RNA dimerization is a prerequisite for RNA packaging in HIV-1, and virion packaging of genomic RNA and RNA dimerization are also linked in other retroviruses. RNA dimers from PR-defective HIV-1 virions are less heat stable than dimers from wild-type mature HIV-1. Similar observations about Moloney murine leukemia virus have also been reported.

Although expression of the Gag-Pol precursor alone is insufficient for production of infectious retroviral particles, the influence of the Gag/Gag-Pol ratio on the viral replication cycle and RNA dimerization is a critical factor. It has been shown that the Gag/Gag-Pol ratio in virion-producing cells is important for the generation of infectious viral particles and the stability of the virion RNA dimer (Xhilaga et al. Journal of Virology, February 2001, p. 1834-1841, Vol. 75, No. 4).

Disclosed herein are packaging systems wherein the ratio of Gag and Gag-Pol proteins is about 99:1, 98:2, 97:3, 96:4, 95:5, 94:6, 93:7, 92:8, 91:9, 90:10, 89:11, 88:12, 87:13, 86:14, 85:15, 84:1,6 83:17, 82:18, 81:19, 80:20, 79:21, 78:22, 77:23, 76:24, 75:25, 74:26, 73:27, 72:28, 71:29, 70:30, 69:31, 68:32, 67:33, 66:34, 65:35, 64:36, 63:37, 62:38, 61:39, 60:40 or any intervening ratio.

In addition, a lentiviral-based packaging construct lacking a nucleic acid sequence capable of expressing the Pol protein, can optionally comprise a nucleic acid sequence capable of expressing a Vpr-Reverse Transcriptase-Integrase protein (in cis). Alternatively, a lentiviral-based packaging system comprising a packaging construct lacking the nucleic acid sequence capable of expressing the Pol protein, can also comprise a separate nucleic acid construct capable of expressing a Vpr-Reverse Transcriptase-Integrase protein (in trans).

The gene transfer constructs disclosed herein can comprise a sequence of interest. The gene transfer construct can also comprise a marker-encoding sequence. For example, the sequence of interest and the marker-encoding sequences can be operably linked to at least one transcriptional control element. Optionally, the gene transfer constructs can comprise two, three, four, five, etc. sequences of interest. The sequences of interest can be the same, or different and can be operably linked to a separate transcriptional control element, or can be operably linked to a transcriptional control element operably linked to another sequence of interest, marker-encoding sequence, or regulator sequence. For example, in the expression systems disclosed herein, the gene transfer construct can comprise a seventh, eighth, ninth or higher ordered nucleic acid sequence, wherein the seventh nucleic acid sequence encodes a third, forth, fifth or higher ordered selected protein of interest.

The gene transfer constructs can further comprise a Woodchuck hepatitis virus posttranscriptional regulatory element located 3′ of the sequence of interest. The gene transfer constructs can also comprise one or more long terminal repeat (LTR) sequences, which are discussed elsewhere herein.

The gene transfer constructs, as well as the other constructs disclosed herein can also be self-inactivating (SIN). SIN vectors are a new generation of retroviral vectors that exploit unique properties of the viral reverse transcriptase enzyme to render some of the cis-acting sequences of an integrated transfer vector proviral DNA inactive. These sequences can include the viral promoter that is found in the LTRs as well as any packaging sequences that are present in the integrated vector proviral DNA. Several strategies to make SIN vectors are available and are well known in the art. For example, the “Split Intron” strategy as described by Tahir A Rizvi in Non-Human Primate Lentiviral Vectors for HumanGene Therapy, Genetic Disorders in the Arab World: United Arab Emirates (available at http://www.cags.org.ae/cbc101v.pdf), which is incorporated herein by reference in its entirety for its teachings of split intron strategy, can be used. The “Split Intron” strategy uses the incorporation of efficient eukaryotic splice sites to delete the packaging sequences from an integrated vector proviral DNA, rendering it incapable of generating an RNA that can be further packaged and propagated by the viral proteins. This eliminates the possibility of any potential recombination of the vector RNA with that of any endogenous or exogenous viruses that can be present fortuitously or otherwise in a retroviral-vector transduced cell. Further, the gene transfer constructs can optionally comprise a mutation in a 3′ long terminal repeat sequence. A promoter sequence can also be substituted for a 5′ or 3′ long terminal repeat sequence.

In addition, the expression systems disclosed herein can include a gene transfer construct that comprises a sequence of interest and a marker-encoding sequence with an element between the sequence of interest and a marker-encoding sequence, wherein the element provides differential expression of the sequence of interest and the marker-encoding sequence. The element between the sequence of interest and the marker-encoding sequence can be an internal ribosomal entry site (IRES) or an internal ribosomal entry site-like element (IRES-like). IRES and IRES-like elements are discussed elsewhere herein. The gene transfer constructs can also comprise at least one transcriptional control element, which are discussed elsewhere herein. The transcriptional control element or elements present in the gene transfer construct can also be regulatable as described elsewhere herein. The gene transfer construct can also comprise a regulator sequence or the regulator sequence can be supplied by a separate regulator construct as described herein.

Further, the gene transfer construct can comprise a marker-encoding sequence and a sequence of interest, wherein the marker-encoding sequence and sequence of interest are operably linked to the same or different transcriptional control element (TCE). For example, disclosed herein are gene transfer constructs wherein the sequence of interest is operably linked to a first transcriptional control element and the marker-encoding sequence is operably linked to a second transcriptional control element. In one example, the first transcriptional control element can be stronger than the second transcriptional control element. In such an arrangement, the expression of the marker-encoding sequence operably linked to the second TCE would be higher than the expression of the sequence of interest operably linked to the second TCE. For example, the ratio of expression between the marker-encoding sequence linked to the first TCE and the sequence of interest operably linked to the second TCE can be 99:1, 98:2, 97:3, 96:4, 95:5, 94:6, 93:7, 92:8, 91:9, 90:10, 89:11, 88:12, 87:13, 86:14, 85:15, 84:1,6 83:17, 82:18, 81:19, 80:20, 79:21, 78:22, 77:23, 76:24, 75:25, 74:26, 73:27, 72:28, 71:29, 70:30, 69:31, 68:32, 67:33, 66:34, 65:35, 64:36, 63:37, 62:38, 61:39, or 60:40.

Furthermore, disclosed are gene transfer constructs comprising two promoters in opposite directions, as well as bidirectional promoters. For example, the sequence of interest and the marker-encoding sequence can be expressed in opposite directions. In another example, the sequence of interest and the marker-encoding sequence can be expressed in opposite directions. Further, the sequence of interest can be operably linked to a first transcriptional control element and the marker-encoding sequence can be operably linked to a second transcriptional control element. The first and second transcriptional control elements can be the same, or can be different. Furthermore, at least one of the transcriptional control elements can be regulatable. Also, the sequence of interest and the marker-encoding sequence can be operably linked to a single transcriptional control element, which can be regulatable. The single transcriptional control element can be a bidirectional promoter that is regulatable.

Specifically disclosed are gene transfer constructs comprising a vector wherein the vector comprises a first nucleic acid sequence, a second nucleic acid sequence, and a third nucleic acid sequence, wherein the first nucleic acid sequence comprises a sequence of interest operably linked to a first transcriptional control element, wherein the second nucleic acid sequence is operably linked to a second transcriptional control element and encodes a polypeptide that controls the expression of the first nucleic acid sequence, wherein the third nucleic acid sequence comprises a regulator sequence operably linked to the first transcriptional control element, and wherein the first and second transcriptional control elements are oriented in opposite directions.

Also disclosed are gene transfer constructs comprising a vector, wherein the vector comprises a first nucleic acid sequence, a second nucleic acid sequence, and a third nucleic acid sequence, wherein the first nucleic acid sequence, the second nucleic acid sequence, and the third nucleic acid sequence are operably linked to single transcriptional control element, wherein the first nucleic acid sequence comprises a sequence of interest, wherein the second nucleic acid sequence encodes a polypeptide that is capable of controlling the expression of the first nucleic acid sequence, wherein the third nucleic acid sequence comprises a regulator sequence operably linked to the first transcriptional control element, and wherein the transcriptional control element is capable of driving expression of the first and second nucleic acid sequences.

The vectors of the gene transfer constructs can be viral vectors and the viral vectors can optionally be self-inactivating. Furthermore, the expression of the first nucleic acid sequences of the gene transfer vectors can be regulatable.

Also disclosed are cells and cell lines that comprise the gene transfer constructs disclosed herein.

Also disclosed are constructs optionally comprising RNA export elements. The term “RNA export element” refers to a cis-acting post-transcriptional regulatory element that regulates the transport of an RNA transcript from the nucleus to the cytoplasm of a cell. Examples of RNA export elements include, but are not limited to, the human immunodeficiency virus (HIV) rev response element (RRE) (see e.g., Cullen et al. (1991) J. Virol. 65: 1053; and Cullen et al. (1991) Cell 58: 423-426), and the hepatitis B virus post-transcriptional regulatory element (PRE) (see e.g., Huang et al. (1995) Molec. and Cell. Biol. 15(7): 3864-3869; Huang et al. (1994) J. Virol. 68(5): 3193-3199; Huang et al. (1993) Molec. and Cell. Biol 13(12): 7476-7486), and U.S. Pat. No. 5,744,326. These references are incorporated herein by reference in their entirety for their teachings of RNA export elements). Generally, the RNA export element is placed within the 3′ UTR of a gene, and can be inserted as one or multiple copies. RNA export elements can be inserted into any or all of the separate vectors generating the packaging cell lines of the present invention.

The constructs disclosed herein can optionally comprise a Tat-encoding nucleic acid sequence. Also disclosed are constructs that can optionally comprise a Rev-encoding nucleic acid sequence. The said Tat and Rev encoding nucleic acid sequences can be either part of or separate from the said Gag or Gag-Pol encoding nucleic acid sequence. The Tat and Rev proteins regulate the levels of HIV gene expression at transcriptional and posttranscriptional levels, respectively. For example, due to the weak basal transcriptional activity of the HIV long terminal repeat (LTR), expression of the provirus initially results in small amounts of multiply spliced transcripts coding for the Tat, Rev, and Nef proteins. Tat increases dramatically HIV transcription by binding to a stem-loop structure (transactivation response element [TAR]) in the nascent RNA, thereby recruiting a cyclin-kinase complex that stimulates transcriptional elongation by the polymerase II complex.

Specifically, Rev is a nucleocytoplasmic shuttle protein that directly binds to its Rev-response element (RRE) RNA target sequence, which is part of all unspliced and incompletely spliced viral mRNAs. Upon multimerization and subsequent interaction with cellular cofactors, Rev promotes the translocation of these mRNAs across the nuclear envelope, leading to the production of the late viral proteins.

Rev accomplishes this effect by serving as a connector between an RNA motif (the RRE), naturally found in the envelope coding region of the HIV transcript, and components of the cell nuclear export machinery. A Rev binding sequence is a nucleic acid which specifically binds to Rev in vitro or in vivo (typically an RNA), or to a nucleic acid which encodes a nucleic acid which binds to Rev in vitro or in vivo (i.e., an RNA or a DNA). Several papers describe in vitro binding assays for monitoring Rev binding, including Wong-Staal et al. (1991) Viral And Cellular Factors that Bind to the Rev Response Element in Genetic Structure and Regulation of HIV (Haseltine and Wong-Staal eds.; part of the Harvard AIDS Institute Series on Gene Regulation of Human Retroviruses, Volume 1), pages 311-322 and the references cited therein, which describe gel mobility-shift assays and footprinting assays for the detection of Rev in biological samples, including human blood. These references are incorporated herein by reference in their entirety for their teachings of binding assays for monitoring Rev binding.

The constructs disclosed herein can optionally comprise a nucleic acid sequence that comprises an RRE. RREs are typically found in the envelope coding region of the HIV transcript and components of the cell nuclear export machinery. As discussed above, upon RRE and Rev multimerization and subsequent interaction with multiple cellular cofactors, translocation of these viral mRNAs across the nuclear envelope can occur.

Also disclosed are Internal Ribosome Entry Sites (IRES) and Internal Ribosome Entry Site-Like elements. Internal Ribosome Entry Sites (IRES) are cis-acting RNA sequences able to mediate internal entry of the 40S ribosomal subunit on some eukaryotic and viral messenger RNAs upstream of a translation initiation codon. Although sequences of IRESs are very diverse and are present in a growing list of mRNAs, IRES elements contain a conserved Yn-Xm-AUG unit (Y, pyrimidine; X, nucleotide), which appears essential for IRES function. Novel IRES sequences continue to be added to public databases every year and the list of unknown IRES sequences is certainly still very large.

IRES-like elements are also cis-acting sequences able to mediate internal entry of the 40S ribosomal subunit on some eukaryotic and viral messenger RNAs upstream of a translation initiation codon. Unlike IRES elements, in IRES-like elements, the Yn-Xm-AUG unit (Y, pyrimidine; X, nucleotide), which appears essential for IRES function, is not required.

The constructs disclosed herein can optionally comprise IRES or IRES-like elements. For example, the packaging constructs disclosed herein can further comprise an element between the first and second nucleic acid sequences wherein the element provides differential expression of the first and second nucleic acid sequences. In a further example, the element between the first and second nucleic acid sequences can be an internal ribosomal entry site or an internal ribosomal entry site-like element. In a further example, the packaging constructs disclosed herein can further comprise an element between the first or second nucleic acid sequences and the third nucleic acid sequence, wherein the third nucleic acid sequence is not located between the first and second nucleic acid sequences, and wherein the element provides differential expression between the first or second nucleic acid sequences and the third nucleic acid sequence.

The IRES or IRES-like element can be naturally occurring or non-naturally occurring. Examples of IRESs include, but are not limited to the IRES present in the IRES database at http://ifr31w3.toulouse.inserm.fr/IRESdatabase/. Examples of IRES can also include, but are not limited to, the EMC-virus IRES, or HCV-virus IRES. In addition, the IRES or IRES-like element can be mutated, wherein the function of the IRES or IRES-like element is retained.

Also disclosed are transcriptional control elements (TCEs). TCEs are elements capable of driving expression of nucleic acid sequences operably linked to them. The constructs disclosed herein comprise at least one TCE. TCEs can optionally be constitutive or regulatable.

Also disclosed are constructs disclosed herein comprising first and second transcriptional control elements oriented in opposite directions wherein the activity of one of the transcriptional control elements can affect the activity of the other transcriptional control elements. Optionally, the two transcriptional control elements can be juxtaposed or a linker sequence can be located between the first and second transcriptional control elements. For example, the linker sequence can be a chromosomal insulator.

Regulatable TCEs can comprise a nucleic acid sequence capable of being bound to a binding domain of a fusion protein expressed from a regulator construct such that the transcription repression domain acts to repress transcription of a nucleic acid sequence contained within the regulatable TCE.

Also disclosed are regulator constructs and regulator sequences. A regulator construct can be a construct comprising a regulator sequence. A regulator sequence can be a sequence that is capable of controlling the expression of a sequence operably linked to a regulator target sequence. For example, a regulator sequence can be a sequence that is capable of encoding a polypeptide that controls the expression of a nucleic acid sequence operably linked to a regulator target sequence in the nucleic acid constructs described elsewhere herein. For example, a regulator construct can be a construct comprising a nucleic acid sequence capable of encoding a drug-controllable (such as a drug inducible) repressor fusion protein that comprises a DNA binding domain and a transcription repression domain. Alternatively, the construct comprising the regulatable TCE can further comprise the nucleic acid sequence capable of encoding a drug-controllable (such as a drug inducible) repressor fusion protein that comprises a DNA binding domain and a transcription repression domain. In such an arrangement, the nucleic acid sequence capable of encoding a drug-controllable (such as a drug inducible) repressor fusion protein is on the same construct as the regulatable TCE to which the repressor fusion protein binds. For example, the packaging constructs and gene transfer constructs can comprise both the nucleic acid sequence capable of encoding a drug-controllable (such as a drug inducible) repressor fusion protein and the regulatable TCE to which the repressor fusion protein binds.

As discussed throughout the specification, the constructs disclosed herein can comprise a regulator sequence, a regulatable TCE comprising a regulator target sequence, or both. The regulator construct can comprise a regulator sequence capable of encoding a tetracycline repressor (tetR) or tetracycline activator (tetA) (otherwise known as reverse tetR-VP16) protein which can bind to a tetO sequence. The tetO sequence can be in a TCE. The regulator construct can optionally comprise a nuclear localization signal-encoding nucleic acid sequence, such as the SV40 nuclear localization signal. For example, the regulator construct can comprise the sequence of SEQ ID NO: 1. Further, the regulator construct can optionally comprise one or more VP16 minimal transactivated domains. For example, the regulator construct can comprise the sequence of SEQ ID NO: 2 or SEQ ID NO: 3. tetR-VP16 can also be referred to as “tet-off”. Reverse tetR-VP16 can also be referred to as “tet-on”.

The regulator constructs can optionally comprise an altered version of tetR and tetA to prevent formation of a heterodimer between the tetR and the tetA proteins. The altered version of tetR and tetA can comprise E and B tet operator DNA binding domains either independently or in combination. For example, the regulator construct can comprise the sequence of SEQ ID NO: 4 or SEQ ID NO: 5.

Regulatable TCEs can optionally comprise a regulator target sequence. Regulator target sequences can comprise nucleic acid sequence capable of being bound to a binding domain of a fusion protein expressed from a regulator construct such that a transcription repression domain acts to repress transcription of a nucleic acid sequence contained within the regulatable TCE. Regulator target sequences can comprise one or more tet operator sequences (tetO). The regulator target sequences can be operably linked to other sequences, including, but not limited to, a TATA box or a GAL-4 encoding nucleic acid sequence.

The gene transfer constructs described herein can optionally comprise a second regulator sequence. For example, a gene transfer construct as described herein can optionally comprise a second GAL-4 encoding nucleic acid sequence operably linked to a second regulator sequence and a second sequence of interest, wherein the second sequence of interest is operably linked to a third transcriptional control element, wherein the second sequence of interest is selected from the group consisting of micro RNA, shRNA, and siRNA, wherein the second regulator sequence is located between the second GAL-4 encoding nucleic acid sequence and the second sequence of interest.

The presence of a regulatable TCE and a regulator sequence, whether they are on the same or a different construct, allows for inducible and reversible expression of the sequences operably linked to the regulatable TCE. As such, the regulatable TCE can provide a means for selectively inducing and reversing the expression of a sequence of interest.

Regulatable TCEs can be regulatable by, for example, tetracycline or doxycycline. Furthermore, the TCEs can optionally comprise at least one tet operator sequence. In one example, at least one tet operator sequence can be operably linked to a TATA box.

Furthermore, the TCE can be a promoter, as described elsewhere herein. Examples of promoters useful with the packaging constructs disclosed herein are given throughout the specification. For example, promoters can include, but are not limited to, CMV based, CAG, SV40 based, heat shock protein, a mH1, a hH1, chicken β-actin, U6, Ubiquitin C, or EF-1α promoters.

Additionally, the TCEs disclosed herein can comprise one or more promoters operably linked to one another, portions of promoters, or portions of promoters operably linked to each other. For example, a transcriptional control element can include, but are not limited to a 3′ portion of a CMV promoter, a 5′ portion of a CMV promoter, a portion of the β-actin promoter, or a 3′CMV promoter operably linked to a CAG promoter.

Preferred promoters controlling transcription from vectors in mammalian host cells can be obtained from various sources, for example, the genomes of viruses such as polyoma, Simian Virus 40 (SV40), adenovirus, retroviruses, hepatitis B virus and most preferably cytomegalovirus, or from heterologous mammalian promoters, e.g., β-actin promoter. The early and late promoters of the SV40 virus are conveniently obtained as an SV40 restriction fragment, which also contains the SV40 viral origin of replication (Fiers et al., Nature, 273: 113 (1978) which is incorporated by reference herein in its entirety for viral promoters). The immediate early promoter of the human cytomegalovirus is conveniently obtained as a HindIII E restriction fragment (Greenway, P. J. et al., Gene 18: 355 360 (1982) which is incorporated by reference herein in its entirety for viral promoters). Of course, promoters from the host cell or related species also are useful herein, and can be used for tissue specific gene expression or tissues specific regulated gene expression. The cited references are incorporated herein by reference in their entirety for their teachings of promoters.

“Enhancer” generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Each of the cited references is incorporated herein by reference in their entirety for their teachings of enhancers. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). Each of the cited references is incorporated herein by reference in their entirety for their teachings of potential locations of enhancers. They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, fetoprotein and insulin), typically one will use an enhancer from a eukaryotic cell virus for general expression. Preferred examples are the SV40 enhancer on the late side of the replication origin (bp 100 270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region are active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

Also disclosed are bidirectional transcriptional control elements. For example, disclosed herein is a bidirectional transcriptional control element comprising a 3′ end of a CMV promoter fused to a 5′ end of a CAG promoter. Also disclosed herein is a bidirectional transcriptional control element comprising a 3′ end of a CMV promoter fused to a 5′ end of a human EF-1α promoter. Also disclosed herein is a bidirectional transcriptional control element comprising the 5′ of a mouse H1 promoter fused to a 5′ end of a CAG promoter. Also disclosed herein is a bidirectional transcriptional control element comprising a 3′ end of a CMV promoter fused to a 5′ end of an SV40 promoter. The bidirectional transcriptional control elements, as the transcriptional control element disclosed elsewhere herein, can be regulatable or constitutive. Also disclosed is a bidirectional transcriptional control element comprising a 5′ end of a CMV promoter fused to a 5′ end of an ef1α promoter.

The bidirectional transcriptional control elements can comprise the sequence set forth in SEQ ID NO: 12, SEQ ID NO: 13, SEQ ID NO: 14, or SEQ ID NO: 51. Bidirectional transcriptional control elements can also comprise regulator target sequences and can be regulated by antibiotics such as tetracycline or doxycycline.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences to improve expression from, or stability of, the construct.

Cre Recombinase is a Type I topoisomerase from bacteriophage P1 that catalyzes the site-specific recombination of DNA between loxP sites (Abremski, K. and Hoess, R. (1984) J. Biol. Chem., 259, 1509-1514, which is incorporated herein by reference in its entirety for its teachings of Cre Recombinase structure and function). The enzyme requires no energy cofactors and Cre-mediated recombination quickly reaches equilibrium between substrate and reaction products (Abremski, K. et al. (1983) Cell, 32, 1301-1311, which is incorporated herein by reference in its entirety for its teachings of the mechanism of action of Cre Recombinase.). The loxP recognition element is a 34 base pair (bp) sequence comprised of two 13 bp inverted repeats flanking an 8 bp spacer region which confers directionality (Metzger, D. and Feil, R. (1999) Curr. Opin. Biotechnol., 10, 470-476, which is incorporated herein by reference in its entirety for its teachings of loxP recognition elements and their role in Cre Recombinase action.). Recombination products depend on the location and relative orientation of the loxP sites. Two DNA species containing single loxP sites can be fused. DNA between directly repeated loxP sites will be excised in circular form while DNA between opposing loxP sites will be inverted with respect to external sequences.

Expression of nucleic acid sequences operably linked to the transcriptional control elements in the gene transfer constructs described herein can also be regulated by Cre recombinase. For example, a gene transfer construct can comprise a vector wherein the vector comprises a first nucleic acid sequence, a second nucleic acid sequence, a third nucleic acid sequence, and a regulator target sequence comprising a nucleic acid sequence capable of encoding a selectable marker, wherein the first nucleic acid sequence comprises a sequence of interest operably linked to a first transcriptional control element, wherein the second nucleic acid sequence is operably linked to a second transcriptional control element and encodes a polypeptide that controls the expression of the first nucleic acid sequence, wherein the third nucleic acid sequence comprises a regulator sequence operably linked to the first transcriptional control element, and wherein the regulator target sequence is also operably linked to the first transcriptional control element and is located between the first transcriptional control element and the first nucleic acid sequence. In such an arrangement, the regulator target sequence can be flanked by TATA sequences, which can be further linked to at least one tet operator sequence. The regulator target sequence with the accompanying sequence can be further flanked by lox P sites, such that, upon Cre-mediated recombination, the regulator target sequence is excised and the sequence of interest can be fused to the first transcriptional control element, allowing expression of the sequence of interest.

Also disclosed herein are packaging constructs wherein the first nucleic acid sequence is operably linked to a first transcriptional control element and the second nucleic acid sequence is operably linked to a second transcriptional control element. Also disclosed are packaging constructs wherein the first and second nucleic acid sequences are operably linked to a first transcriptional control element and the third nucleic acid sequence is operably linked to a second transcriptional control element.

Optionally, the first transcriptional control element can be stronger than the second transcriptional control element. In such an arrangement, the expression of the sequence or sequences operably linked to the first TCE would be higher than the expression of the sequence or sequences operably linked to the second TCE. For example, the ratio of expression between the sequence or sequences operably linked to the first TCE and the sequence or sequences operably linked to the second TCE can be about 99:1, 98:2, 97:3, 96:4, 95:5, 94:6, 93:7, 92:8, 91:9, 90:10, 89:11, 88:12, 87:13, 86:14, 85:15, 84:1,6 83:17, 82:18, 81:19, 80:20, 79:21, 78:22, 77:23, 76:24, 75:25, 74:26, 73:27, 72:28, 71:29, 70:30, 69:31, 68:32, 67:33, 66:34, 65:35, 64:36, 63:37, 62:38, 61:39, 60:40 or any intervening ratio.

Further disclosed are packaging constructs comprising two promoters in opposite directions, as well as bidirectional promoters. For example, the first and the second nucleic acid sequences can be expressed in opposite directions. In another example, the first and second nucleic acid sequences can be expressed in the opposite direction of the third nucleic acid sequence. Optionally, the marker-encoding sequence and the gene of interest can be expressed in opposite directions. Further, the first nucleic acid sequence can be operably linked to a first transcriptional control element and the second nucleic acid sequences can be operably linked to a second transcriptional control element. Further, the first and second nucleic acid sequences can be operably linked to a first transcriptional control element and the third nucleic acid sequence can be operably linked to a second transcriptional control element. The first and second transcriptional control elements can be the same, or can be different. Furthermore, at least one of the transcriptional control elements can be regulatable. Also, the first and second nucleic acid sequences can be operably linked to a single transcriptional control element, which can be regulatable. Further, the first, second and third nucleic acid sequences can be operably linked to a single transcriptional control element, which can be regulatable. The single transcriptional control element can also be a bidirectional promoter, which can also be regulatable.

A typical promoter consists of a minimal promoter and other upstream cis elements. Lewin, B. Gene VI (Oxford University Press, Oxford, 1997), Odell, J. T., Nagy, F. & Chua, N.-H. Nature 313, 810-812 (1990), and Benfey, P. N. & Chua, N.-H. Science 250, 959-966 (1990). The minimal promoter is essentially a TATA box region where RNA polymerase binds to initiate transcription, but itself has no transcriptional activity. Benfey, P. N. & Chua, N.-H. Science 250, 959-966 (1990). The cis elements, upon binding by specific transcriptional factors, individually or in combination, determine the spatio-temporal expression pattern of a promoter. (Benfey, P. N. & Chua, N.-H. Science 250, 959-966 (1990).) U.S. Pat. No. 5,814,618 discloses a bidirectional promoter which has multiple tet operator sequences (defined in the specification as enhancers or repressors) and flanking minimal promoters. U.S. Pat. No. 5,955,646 discloses bidirectional heterologous constructs. U.S. Pat. No. 5,368,855 discloses a naturally-occurring bidirectional promoter. U.S. Pat. No. 5,359,142 discloses constructs which have been manipulated to permit variation in enhancement of gene expression. U.S. Pat. No. 5,627,046 discloses a naturally-occurring bidirectional promoter. U.S. Pat. No. 5,827,693 discloses modified hemoglobin promoters. All of these references are herein incorporated by reference in their entirety regarding their teaching of bidirectional promoters.

Also disclosed herein are packaging constructs comprising one or more mutations in the nucleic acid sequences encoding Gag and Gag-Pro-Pol proteins that reduce frame-shifting or translational read-through required for the synthesis of Gag-Pro and Gag-Pro-Pol proteins. Also disclosed are packaging constructs comprising one or more mutations that reduce frame-shifting or translational read-through required for the synthesis of Gag-Pro and Gag-Pro proteins.

Gag and Gag-Pol are naturally made from the same mRNA transcript at a molar ratio of approximately 20:1 in HIV type 1 (HIV-1) and SIV-infected cells. This ratio is achieved by ribosomal frameshifting or read-through in the region of overlap between the gag and pol or gag and pro reading frames (Swanstrom, R., and J. W. Wills. 1997. Synthesis, assembly, and processing of viral proteins, p. 263-334. In J. M. Coffin, S. H. Hughes, and H. E. Varmus (ed.), Retroviruses. Cold Spring Harbor Laboratory Press, Cold Spring Harbor, N.Y., which is incorporated herein by reference in their entireties for its teachings of frameshifting). As the precursor to the catalytic subunits of mature virions, Pol is essential for virion maturation and infectivity and its incorporation into assembling virus particles is dependent on its association with Gag (Id.). The gag-pol frameshift site consists of a conserved seven-nucleotide slippery sequence (UUUUUUA) SEQ ID NO: 7 followed immediately downstream by a region of RNA secondary structure (Swanstrom, R., and J. W. Wills. 1997. Synthesis, assembly, and processing of viral proteins, p. 263-334. In J. M. Coffin, S. H. Hughes, and H. E. Varmus (ed.), Retroviruses. Cold Spring Harbor Laboratory Press, Cold Spring Harbor, N.Y.). Ribosomal frameshifting physically occurs within the slippery sequence when the tRNAs for phenylalanine and leucine (codons UUU UUA; SEQ ID NO: 8 slip back one nucleotide (−1) relative to the gag frame (UUU UUA; SEQ ID NO: 8)→UUU UUU (SEQ ID NO: 9)) and translation continues in the pol reading frame (Jacks, T., M. D. Power, F. R. Masiarz, P. A. Luciw, P. J. Barr, and H. E. Varmus. 1988. Characterization of ribosomal frameshifting in HIV-1 gag-pol expression. Nature 331:280-283, which is incorporated herein by reference in their entireties for its teachings of ribosomal frameshifting in HIV-1). For example, the mutation can disrupt the loop structure required for frame-shifting. This can be accomplished by altering or removing the individual nucleotides to disrupt loop structure.

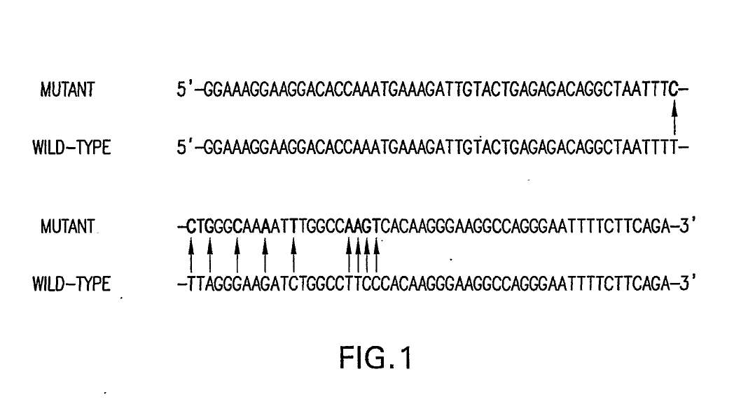

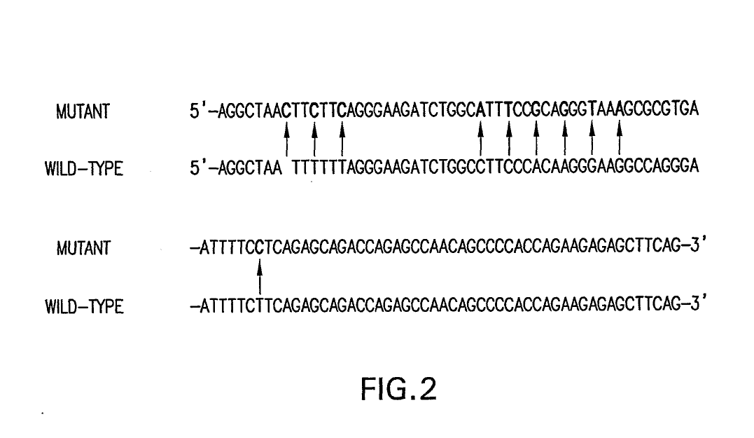

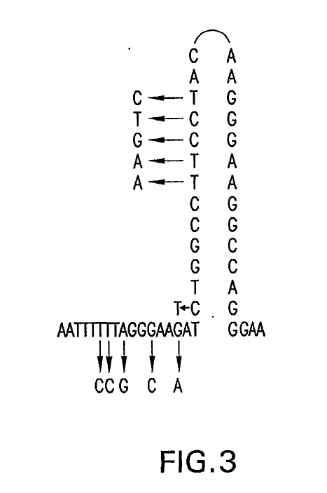

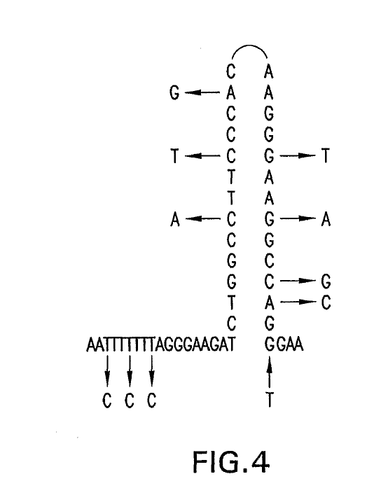

For example, FIG. 3 shows the loop structure in HIV gag and HIV gag-pol required for frame-shifting. FIG. 4 shows an altered sequence of loop structure in HIV gag and HIV gag-pol required for frame-shifting that results in the disruption of the loop structure required for frame-shifting. Disclosed are packaging constructs wherein the first nucleic acid sequence comprises mutations in the gag and gag-pol sequences required for frame-shifting. Optionally, the gag sequence required for frame-shifting can comprise point mutations. For example, the gag sequence required for frame-shifting can comprise point mutations as presented in FIG. 1. Optionally, the first nucleic acid sequence can comprise the nucleotide sequence of SEQ ID NO: 10. The second nucleic acid sequence, for example, can comprise a single nucleotide insertion as well as several point mutations as presented in FIG. 2. Optionally, the second nucleic acid sequence can comprise the nucleotide sequence of SEQ ID NO: 11.

Codon preference among different species can be dramatically different. To enhance the expression level of a foreign protein in a particular expression system (E. coli, yeast, insect, or mammalian cell), it can be very important to adjust the codon frequency of the foreign protein to match that of the host expression system. This process is known as codon-optimization. Codon-optimization refers to the alteration of gene sequences to make codon usage match the available tRNA pool within the cell/species of interest. Codon-optimization has emerged as a powerful tool to increase protein expression by genes from small RNA and DNA viruses, which commonly contain overlapping reading frames as well as structural elements that are embedded within coding regions; these features are not widespread among large DNA viruses.

Immunization with codon-optimized env (Andre, S., B. Seed, J. Eberle, W. Schraut, A. Bultmann, and J. Haas. 1998. Increased immune response elicited by DNA vaccination with a synthetic gp120 sequence with optimized codon usage. J. Virol. 72:1497-1503.) and gag (Deml, L., A. Bojak, S. Steck, M. Graf, J. Wild, R. Schirmbeck, H. Wolf, and R. Wagner. 2001. Multiple effects of codon usage optimization on expression and immunogenicity of DNA candidate vaccines encoding the human immunodeficiency virus type 1 Gag protein. J. Virol. 75:10991-11001; zur Megede, J., M. C. Chen, B. Doe, M. Schaefer, C. E. Greer, M. Selby, G. R. Otten, and S. W. Barnett. 2000. Increased expression and immunogenicity of sequence-modified human immunodeficiency virus type I gag gene. J. Virol. 74:2628-2635) genes of human immunodeficiency virus type 1 (HIV-1) led to enhanced expression of the genes and improved immune responses against the antigens. Similar studies conducted with a variety of other pathogenic organisms, such as Listeria (Nagata, T., M. Uchijima, A. Yoshida, M. Kawashima, and Y. Koide. 1999. Codon optimization effect on translational efficiency of DNA vaccine in mammalian cells: analysis of plasmid DNA encoding a CTL epitope derived from microorganisms. Biochem. Biophys. Res. Commun. 261:445-451), bacteria producing tetanus toxin (Stratford, R., G. Douce, L. Zhang-Barber, N. Fairweather, J. Eskola, and G. Dougan. 2000. Influence of codon usage on the immunogenicity of a DNA vaccine against tetanus. Vaccine 19:810-815), Plasmodium (Nagata, T., M. Uchijima, A. Yoshida, M. Kawashima, and Y. Koide. 1999. Codon optimization effect on translational efficiency of DNA vaccine in mammalian cells: analysis of plasmid DNA encoding a CTL epitope derived from microorganisms. Biochem. Biophys. Res. Commun. 261:445-451), human papillomavirus (Cid-Arregui, A., V. Juarez, and H. zur Hausen. 2003. A synthetic E7 gene of human papillomavirus type 16 that yields enhanced expression of the protein in mammalian cells and is useful for DNA immunization studies. J. Virol. 77:4928-4937; Liu, W., F. Gao, K. Zhao, W. Zhao, G. Fernando, R. Thomas, and I. Frazer. 2002. Codon modified human papillomavirus type 16 E7 DNA vaccine enhances cytotoxic T-lymphocyte induction and anti-tumour activity. Virology 301:43-52), and others (Gurunathan, S., D. M. Klinman, and R. A. Seder. 2000. DNA vaccines: immunology, application, and optimization. Annu. Rev. Immunol. 18:927-974), ascertained the potential of codon optimization to enhance the efficiency of the DNA vaccines. Codon optimization can be performed using a variety of techniques known by one of skill in the art. For example, the method described in Ramakrishna L, Anand K K, Mohankumar K M, Ranga U. J. Virol. 2004 September; 78(17):9174-89 can be used. All of the cited references are incorporated by reference herein in their entirety for their teachings of codon optimization.

Also disclosed herein are packaging constructs where codon-optimization has been employed. For example, the packaging constructs described herein can be modified so that the first nucleic acid sequence is codon optimized. In another embodiment, the second nucleic acid sequence can be codon optimized. Also, both the first and the second nucleic acids can be codon optimized.

Also disclosed herein are packaging constructs wherein the construct is capable of generating non-replication competent recombinants. Also, disclosed herein are packaging constructs wherein the construct is not capable of generating replication competent recombinants. As discussed above, in view of the advantages associated with retroviral vectors, particularly lentiviruses which are capable of infecting non-dividing cells, improved methods for generating pure stocks of recombinant virus, free of replication-competent helper virus, have been the subject of much investigation. Recombinant retroviruses are generally produced by introducing a suitable proviral DNA vector into mammalian cells (“packaging cells”) that produce the necessary viral proteins for encapsidation of the desired recombinant RNA, but which lack the signal for packaging viral RNA (ψ sequence). Thus, while the required gag, pol, and env genes of the retrovirus are intact, there is no release of wild-type helper virus by these packaging lines. However, when the cells are transfected with a separate vector containing the v sequence required for packaging, wild-type retrovirus can arise by recombination (Mann et al. (1983) Cell 33:153). This can represent a significant safety hazard, particularly in the case of lentiviruses, such as HIV, and for certain application of the vector, such as gene therapy.

Current approaches to avoid the dangers associated with recombination leading to production of replication-competent helper virus include making additional mutations (e.g., LTR deletions) in the viral constructs used to create packaging lines, and separating the viral genes necessary for producing virions onto separate plasmids. For example, it has recently been shown that recombinant Moloney murine leukemia virus (MuLV), free of detectable helper-virus, can be produced by separating the gag and pol genes from the env gene in packaging cells (Markowitz et al. (1998) J. Virol. 62(4):1120). These packaging cells contained two separate plasmids collectively encoding the viral proteins necessary for virion production, reducing the likelihood that the recombination events necessary to produce intact retrovirus (i.e., between three plasmid vectors) would occur when cotransfected with a third vector containing the V packaging signal.

The constructs disclosed herein can optionally comprise a nuclear localization signal-encoding nucleic acid sequence. In addition the constructs disclosed herein can optionally comprise a nuclear localization signal-encoding nucleic acid sequence operably linked to a tetracycline transactivator-encoding nucleic acid. For example, the constructs disclosed herein can comprise a nuclear localization signal-encoding nucleic acid sequence operably linked to a tetracycline transactivator-encoding nucleic acid, such as tet-on. A nuclear localization sequence is one that directs a polypeptide from the cytoplasm to the nuclear membrane and hence the nucleus. The nuclear localization signal-encoding nucleic acid can further comprise a transcriptional control element. Transcriptional control elements are disclosed elsewhere herein. The nuclear localization signal-encoding nucleic acid sequence can also be flanked by at least one linker sequence, which can, for example, encode SEQ ID NO: 15 (GGGGS), which comprises four glycine residues followed by a serine residue. A linker sequence can be a chromosomal insulator and can also be a generic sequence. Generally, the linker sequence serves to reduce interference of each functional domain of the fusion protein. For example, the linker sequence can reduce interference with the tet R or tetA proteins, SV40 NLS, VP16, or with the ZNF10 silencing protein. A linker that is a chromosomal insulator can reduce the interference between the inducible promoter and the constitutive promoter of the constructs disclosed herein, thereby reducing leakage of the inducible promoter.

Also disclosed are cell lines comprising the packaging constructs disclosed herein. Methods for producing cell lines are also described elsewhere herein.

The embodiments described above and below are useful with any of the compositions and methods disclosed herein.

### Systems

Also disclosed herein are packaging systems useful with the packaging constructs discussed above. For example, a packaging system can comprise the packaging constructs of the invention and a nucleic acid construct that expresses an envelope glycoprotein, as discussed elsewhere herein. Also disclosed are packaging cell lines. Packaging cell lines for producing viral-like particles comprise a target cell and one of the packaging constructs disclosed herein. Packaging cell lines can also comprise a nucleic acid construct that expresses an envelope glycoprotein, as discussed elsewhere herein. As used herein, an envelope glycoprotein permits pseudotyping of particles generated by the packaging construct. Constructs comprising a nucleic acid sequence that is capable of expressing an envelope glycoprotein is described herein. For example, the envelope constructs can include the G glycoprotein of vesicular stomatitis virus (VSV G) and the envelope of the Moloney leukemia virus (MLV).

Also disclosed herein are packaging and expression systems wherein the packaging constructs comprising nucleic acids for Gag and Gag-Pro-Pol in trans. For example, disclosed is an expression system comprising a first, second, and third packaging construct. The first packaging construct comprises a first nucleic acid construct comprising a nucleic acid sequence that encodes a Gag polyprotein, wherein the Gag-encoding nucleic acid sequence comprises one or more mutations that reduce frame-shifting or translational read-through and is operably linked to at least one transcriptional control element. The second packaging construct comprises a second nucleic acid construct comprising a nucleic acid sequence that encodes a Gag-Pro-Pol protein, wherein the Gag-Pro-Pol-encoding nucleic acid sequence comprises one or more mutations that reduce frame-shifting or translational read-through and is operably linked to at least one transcriptional control element. The third nucleic acid construct comprises a third nucleic acid sequence that encodes an envelope glycoprotein, wherein the third nucleic acid sequence is operably linked to at least one transcriptional control element. The packaging and expression systems comprising these constructs can also comprise a gene transfer construct comprising at least one gene of interest.

Also disclosed is a packaging system comprising a first nucleic acid construct comprising a first mutated nucleic acid that encodes a Gag polyprotein, wherein the first mutated nucleic acid is operably linked to a transcriptional control element; and a second nucleic acid construct comprising a second mutated nucleic acid that encodes a Gag-Pol polyprotein, wherein the second mutated nucleic acid is operably linked to a transcriptional control element. The mutations in the first and second nucleic acid constructs can result in a ratio of the expression of the Gag and Gag-Pol polyproteins that allow viral particle formation. Optionally, the first mutated nucleic acid of the packaging system can be operably linked to a minimal CMV promoter and the second mutated nucleic acid can be operably linked to the heat shock protein promoter. Other promoters suitable for use with the constructs of the packaging system are described elsewhere herein.

The constructs and viral particles of the present invention can be used, in vitro, in vivo and ex vivo, to introduce sequences of interest into a target cell (e.g., a eukaryotic cell) or a mammal (e.g., a human or other mammal or vertebrate). The cells can be obtained commercially or from a depository or obtained directly from a mammal, such as by biopsy. The cells can be obtained from a mammal to whom they will be returned or from another/different mammal of the same or different species. For example, using the packaging cell lines or viral particles of the present invention, DNA of interest can be introduced into nonhuman cells, such as pig cells, which are then introduced into a human. Alternatively, the cell need not be isolated from the mammal where, for example, it is desirable to deliver viral particles of the present invention to the mammal in gene therapy.

Ex vivo therapy has been described, for example, in Kasid et al., Proc. Natl. Acad. Sci. USA, 87:473 (1990); Rosenberg et al., N. Engl. J. Med., 323:570 (1990); Williams et al., Nature, 310:476 (1984); Dick et al., Cell, 42:71 (1985); Keller et al., Nature, 318:149 (1985); and Anderson et al., U.S. Pat. No. 5,399,346, are incorporated by reference herein in their entirety for their teachings of ex vivo therapy.

Methods for administering (introducing) viral particles directly to a mammal are generally known to those practiced in the art. For example, modes of administration include parenteral, injection, mucosal, systemic, implant, intraperitoneal, oral, intradermal, transdermal (e.g., in slow release polymers), intramuscular, intravenous including infusion and/or bolus injection, subcutaneous, topical, epidural, etc. Viral particles of the present invention can, preferably, be administered in a pharmaceutically acceptable carrier, such as saline, sterile water, Ringer's solution, and isotonic sodium chloride solution.

The dosage of a viral particle of the present invention administered to a mammal, including frequency of administration, will vary depending upon a variety of factors, including mode and route of administration; size, age, sex, health, body weight and diet of the recipient mammal; nature and extent of symptoms of the disease or disorder being treated; kind of concurrent treatment, frequency of treatment, and the effect desired.

Disclosed are expression systems comprising a packaging construct as described herein, wherein the expression system also comprises an envelope nucleic acid construct comprising an envelope glycoprotein-encoding nucleic acid sequence, wherein the envelope glycoprotein-encoding nucleic acid sequence is operably linked to at least one transcriptional control element; and also comprises a gene transfer construct comprising one or more sequences of interest. Also disclosed are expression systems, wherein an envelope glycoprotein promotes entry into a cell. Optionally, the envelope glycoprotein can be a viral envelope glycoprotein, such as the G protein of vesicular stomatitis virus (VSV-G), or one of several other viral glycoproteins that are know in the art to mediate entry into a cell.

Optionally, the expression system can further comprise a nuclear localization signal-encoding construct comprising a nuclear localization signal-encoding nucleic acid sequence operably linked to a tetracycline transactivator-encoding nucleic acid, as disclosed above. Nuclear localization sequences are disclosed above. For example, the nuclear localization signal-encoding construct can also comprise from 5′ to 3′ a Cytomegalovirus promoter, a first linker encoding sequence, a second nuclear localization signal, a second linker sequence, and a tetracycline transactivator-encoding sequence, wherein the encoded linker is GGGGS (SEQ ID NO: 15).

Also disclosed are expression systems comprising a first packaging construct, wherein the first packaging construct comprises a first nucleic acid construct comprising a nucleic acid sequence that encodes a Gag polyprotein, wherein the Gag-encoding nucleic acid sequence comprises one or more mutations that reduce frame-shifting or translational read-through and is operably linked to at least one transcriptional control element and also comprising a second packaging construct comprising a second nucleic acid construct comprising a nucleic acid sequence that encodes a Gag-Pol polyprotein, wherein the Gag-Pol-encoding nucleic acid sequence comprises one or more mutations that reduce frame-shifting or translational read-through and is operably linked to at least one transcriptional control element. The expression system also comprises a third nucleic acid construct comprising a third nucleic acid sequence that encodes an envelope glycoprotein, wherein the third nucleic acid sequence is operably linked to at least one transcriptional control element. The expression system also comprises a gene transfer construct comprising one or more sequences of interest. Optionally, the expression system can further comprise a nuclear localization signal-encoding construct comprising a nuclear localization signal-encoding nucleic acid sequence operably linked to a tetracycline transactivator-encoding nucleic acid. A nuclear localization sequence is one which directs a polypeptide from the cytoplasm to the nuclear membrane and hence the nucleus.

The expression systems disclosed above can also comprise a fourth nucleic acid construct comprising a fourth nucleic acid sequence that encodes a nuclear localization signal operably linked to a tetracycline transactivator. The fourth nucleic acid construct can further comprise a transcriptional control element, such as a promoter, for example. The nuclear localization signal-encoding sequence can also be flanked by at least one linker sequence. The fourth nucleic acid sequence can also comprise a 5′ to 3′ a Cytomegalovirus promoter, a nucleic acid sequence encoding SEQ ID NO: 15 (GGGGS), a nucleic acid sequence encoding a nuclear localization signal, a nucleic acid sequence encoding SEQ ID NO: 15 (GGGGS) and a nucleic acid sequence encoding a tetracycline transactivator.

Also disclosed are cell lines comprising the expression systems disclosed elsewhere herein.

Also disclosed are envelope nucleic acid constructs comprising an envelope glycoprotein-encoding nucleic acid sequence, wherein the envelope glycoprotein-encoding nucleic acid sequence is operably linked to at least one transcriptional control element. The envelope glycoprotein can promote entry into a cell. The envelope glycoprotein can optionally be viral. In one example, the envelope glycoprotein can be a G protein of vesicular stomatitis virus (VSV-G).

Also disclosed are embodiments wherein cis-acting elements are required for encapsidation, reverse transcription and integration. The cis-acting elements can be provided in trans or in cis with the constructs described herein. For example, the packaging construct lacking the nucleic acid sequence capable of expressing the Pol protein, can optionally comprise a nucleic acid sequence capable of expressing a Vpr-Reverse Transcriptase-Integrase protein (in cis). Alternatively, a packaging system comprising the packaging construct lacking the nucleic acid sequence capable of expressing the Pol protein, can also comprise a separate nucleic acid construct capable of expressing a Vpr-Reverse Transcriptase-Integrase protein (in trans).

Also disclosed is a gene transfer method comprising introducing into a cell a packaging nucleic acid construct described elsewhere herein, and introducing to the cell an envelope construct comprising a nucleic acid sequence that encodes an envelope glycoprotein, wherein the envelope glycoprotein encoding nucleic acid sequence is operably linked to at least one transcriptional control element and introducing into the cell a gene transfer construct described elsewhere herein comprising one or more sequences of interest; and maintaining the cell under conditions that allow formation of a virus-like particle, the virus-like particle contains containing the gene(s) or sequence(s) of interest.

Also disclosed is a cell comprising an exogenous sequence of interest, where the sequence of interest is transferred into the cell using the gene transfer method described above.

The constructs described herein can optionally include a nucleic acid sequence encoding a marker product. This marker product is used to determine if the gene has been delivered to the cell and once delivered is being expressed. Preferred marker genes are the E. Coli lacZ gene, which encodes B galactosidase, and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR cells and mouse LTK cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells that were not transformed with the DHFR or TK gene will not be capable of survival in non-supplemented media.

The second category is dominant selection, which refers to a selection scheme used in any cell type and does not require the use of a mutant cell line. These schemes typically use a drug to arrest growth of a host cell. Those cells that have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410 413 (1985. These)). The cited references are incorporated herein by reference herein in their entirety for their teachings of examples of dominant selection. The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

Also disclosed are envelope nucleic acid constructs comprising an envelope glycoprotein-encoding nucleic acid sequence, wherein the envelope glycoprotein-encoding nucleic acid sequence is operably linked to at least one transcriptional control element. The envelope glycoprotein can promote entry into a cell. The envelope glycoprotein can optionally be viral. In one example, the envelope glycoprotein can be a G protein of vesicular stomatitis virus (VSV-G).

### Methods

Disclosed herein are methods of making virus-like particles. For example, a method of making virus-like particles comprises using the packaging constructs of the invention. Also disclosed are methods of making a virus-like particle, comprising introducing any of the packaging nucleic acid constructs described above into a cell; and introducing to the cell an envelope construct comprising a nucleic acid sequence that encodes an envelope glycoprotein, wherein the envelope glycoprotein encoding nucleic acid sequence is operably linked to at least one transcriptional control element; and maintaining the cell under conditions that allow formation of a virus-like particle.

Further disclosed herein are methods of making a virus-like particle, comprising introducing any of the packaging nucleic acid constructs described above into a cell; and introducing into the cell an envelope construct comprising a nucleic acid sequence that encodes an envelope glycoprotein, wherein the envelope glycoprotein encoding nucleic acid sequence is operably linked to at least one transcriptional control element; and maintaining the cell under conditions that allow formation of a virus-like particle.

Virus-like particles can be prepared by inserting selected lentiviral sequences into a suitable vector (e.g., a commercially available expression plasmid containing appropriate regulatory elements (e.g., a promoter and enhancer), restriction sites for cloning, marker genes etc.). This can be achieved using standard cloning techniques, including PCR, as is well known in the art. Lentiviral sequences to be cloned into such vectors can be obtained from any known source, including lentiviral genomic RNA, or cDNAs corresponding to viral RNA. Suitable cDNAs corresponding to lentiviral genomic RNA are commercially available and include, for example, pNLENV-1 (Maldarelli et al. (1991) J. Virol. 65:5732) which contains genomic sequences of HIV-1, which is incorporated by reference herein in its entirety for its teachings of suitable cDNAs corresponding to lentiviral genomic RNA. Other sources of retroviral (e.g., lentiviral) cDNA clones include the American Type Culture Collection (ATCC), Rockville, Md. These references are incorporated herein by reference in their entirety for their teachings of examples of cDNAs corresponding to lentiviral genomic RNA that are currently available., these clones are incorporated by reference herein in their entirety for examples of retroviral cDNA clones that can be used in the compositions and methods disclosed herein.

Once cloned into an appropriate vector (e.g., expression vector), retroviral sequences (e.g., gag, pol, env, LTRs and cis-acting sequences) can be modified as described herein. In one embodiment, lentiviral sequences amplified from plasmids, such as pNLENV-1, can be cloned into a suitable backbone vector, such as a pUC vector (e.g., pUC19) (University of California, San Francisco), pBR322, or pcDNA1 (Invitrogen, Inc., Carlsbad, Calif.), and then modified by deletion (using restriction enzymes), substitution (e.g., using site directed mutagenesis), or other (e.g., chemical) modification to prevent expression or function of selected lentiviral sequences. As described herein, portions of the gag, pol and env genes can be removed or mutated, along with selected accessory genes. For example, in one embodiment, the nucleic acid sequences encoding Gag and Gag-Pro-Pol polyproteins are mutated so as to reduce frame-shifting or translational read-through required for the synthesis of Gag-Pro and Gag-Pro-Pol polyproteins.

Each vector of the invention can contain the minimum lentiviral sequences necessary to encode the desired lentiviral proteins (e.g., gag, pol and env) or direct the desired lentiviral function (e.g., packaging of RNA). That is, the remainder of the vector is preferably of non-viral origin, or from a virus other than a lentivirus (e.g., HIV). In one embodiment, lentiviral LTRs contained in the retroviral vectors of the invention are modified by replacing a portion of the LTR with a functionally similar sequence from another virus, creating a hybrid LTR. For example, the lentiviral 5′LTR, which serves as a promoter, can be partially replaced by the CMV promoter or an LTR from a different retrovirus (e.g., MuLV or MuSV). Alternatively, or additionally, the lentiviral 3′ LTR can be partially replaced by a polyadenylation sequence from another gene or retrovirus. Optionally, a portion of the HIV-1 3′ LTR is replaced by the polyadenylation sequence of the rabbit β-globin gene. By minimizing the total lentiviral sequences within the vectors of the invention in this manner, the chance of recombination among the vectors, leading to replication-competent helper lentivirus, is greatly reduced.

Any suitable expression vector can be employed in the present invention. As described herein, suitable expression constructs can include a human cytomegalovirus (CMV) immediate early promoter construct. The cytomegalovirus promoter can be obtained from any suitable source. For example, the complete cytomegalovirus enhancer-promoter can be derived from the human cytomegalovirus (hCMV). Other suitable sources for obtaining CMV promoters include commercial sources, such as Clontech (Mountain View, Calif.), Invitrogen (Carlsbad, Calif.) and Stratagene (La Jolla, Calif.). Part, or all, of the CMV promoter can be used in the present invention. Other examples of constructs which can be used to practice the invention include constructs that use MuLV, SV40, Rous Sarcoma Virus (RSV), vaccinia P7.5, heat shock, and rat β-actin promoters. In some cases, such as the RSV and MuLV, these promoter-enhancer elements are located within or adjacent to the LTR sequences.

Suitable regulatory sequences required for gene transcription, translation, processing and secretion are recognized in the art, and are selected to direct expression of the desired protein in an appropriate cell. Accordingly, the term “regulatory sequence” as used herein, includes any genetic element present 5′ (upstream) or 3′ (downstream) of the translated region of a gene and which control or affect expression of the gene, such as enhancer and promoter sequences. Such regulatory sequences are discussed, for example, in Goeddel, Gene expression Technology: Methods in Enzymology, page 185, Academic Press, San Diego, Calif. (1990), which is incorporated by reference herein in their entirety for its teachings of regulatory sequences. Regulatory sequences can be selected by those of ordinary skill in the art for use in the present invention.

In one embodiment, the invention employs an inducible promoter within the constructs disclosed herein, so that transcription of selected genes can be turned on and off. This minimizes cellular toxicity caused by expression of cytotoxic viral proteins, increasing the stability of the packaging cells containing the vectors. For example, high levels of expression of VSV-G (envelope protein) and Vpr can be cytotoxic (Yee, J.-K., et al., Proc. Natl. Acad. Sci., 91:9654-9568 (1994) and, therefore, expression of these proteins in packaging cells of the invention can be controlled by an inducible operator system, such as the inducible Tet operator system (GIBCO BRL, Carlsbad, Calif.), allowing for tight regulation of gene expression (i.e., generation of retroviral particles) by the concentration of tetracycline in the culture medium. That is, with the Tet operator system, in the presence of tetracycline, the tetracycline is bound to the Tet transactivator fusion protein (tTA), preventing binding of tTA to the Tet operator sequences and allowing expression of the gene under control of the Tet operator sequences (Gossen et al. (1992) PNAS 89:5547-5551), which is incorporated by reference herein in their entirety for its teachings of the tTA and allowing expression of the gene under control of the Tet operator sequences. In the absence of tetracycline, the tTA binds to the Tet operator sequences preventing expression of the gene under control of the Tet operator.

Examples of other inducible operator systems that can be used for controlled expression of the protein, wherein the protein provides for a pseudotyped envelope are 1) inducible eukaryotic promoters responsive to metal ions (e.g., the metallothionein promoter), glucocorticoid hormones and 2) the LacSwitch™ Inducible Mammalian Expression System (Stratagene) (La Jolla, Calif.) of E. coli. Briefly, in the E. coli lactose operon, the Lac repressor binds as a homotetramer to the lac operator, blocking transcription of the lac2 gene. Inducers such as allolactose (a physiologic inducer) or isopropyl-β-D-thiogalactoside (IPTG, a synthetic inducer) bind to the Lac repressor, causing a conformational change and effectively decreasing the affinity of the repressor for the operator. When the repressor is removed from the operator, transcription from the lactose operon resumes.

Also disclosed herein are methods of selectively regulating the expression of a sequence of interest comprising introducing a gene transfer construct, as described herein, to a target cell under conditions suitable to allow regulation of the sequence of interest. The methods disclosed herein can also be used to direct the expression of a sequence of interest in a tissue-specific manner. For example, a gene transfer construct can comprise a tissue specific TCE that can be used to drive expression of a sequence of interest in a specific tissue. Such a gene transfer vector can be used in combination with the packaging constructs to make viral particles as described herein. The viral particles can then be introduced into a zygote. Optionally, tissue specific expression can be achieved using the methods disclosed herein for generation of transgenic animals, wherein expression of the sequence of interest is under the control of an inducible/reversible TCE. In such an animal, expression of the sequence of interest can be limited to a site where, for example, DOX is administered. As such, expression of a sequence of interest will only occur at the site of DOX administration.

Also disclosed herein are methods of administering to a subject the viral particles generated using the methods of the invention. The constructs and viral particles of the present invention can be used, in vitro, in vivo and ex vivo, to introduce sequences of interest into a target cell (e.g., a mammalian cell) or a mammal (e.g., a human). The cells can be obtained commercially or from a depository or obtained directly from a mammal, such as by biopsy. The cells can be obtained from a mammal to whom they will be returned or from another/different mammal of the same or different species. For example, using the packaging cell lines or viral particles of the present invention, DNA of interest can be introduced into nonhuman cells, such as pig cells, which are then introduced into a human. Alternatively, the cell need not be isolated from the mammal where, for example, it is desirable to deliver viral particles of the present invention to the mammal in gene therapy.

Ex vivo therapy has been described, for example, in Kasid et al., Proc. Natl. Acad. Sci. USA, 87:473 (1990); Rosenberg et al., N. Engl. J. Med., 323:570 (1990); Williams et al., Nature, 310:476 (1984); Dick et al., Cell, 42:71 (1985); Keller et al., Nature, 318:149 (1985); and Anderson et al., U.S. Pat. No. 5,399,346, which are incorporated herein by reference in their entirety for their teachings of ex vivo therapy.

Also disclosed herein are methods of administering to a subject the viral particles generated using the methods of the invention. Traditionally, successful antiviral vaccines have relied mostly on live-attenuated viruses. Live-attenuated HIV vaccine candidates are not ideal as they pose risks of reversion, recombination or mutations. Other current HIV vaccine candidates have difficulties generating broadly effective neutralising antibodies and cytotoxic T cell immune responses to primary HIV isolates. Virus-like-particles (VLPs) have been demonstrated to be safe to administer to animals and human patients as well as being potent and efficient stimulators of cellular and humoral immune responses. Therefore, VLPs are useful as HIV vaccines. Chimeric HIV-1 VLPs constructed with either HIV or SIV capsid protein plus HIV immune epitopes and immuno-stimulatory molecules have further improved on early VLP designs, leading to enhanced immune stimulation. The administration of VLP vaccines via mucosal surfaces has also emerged as a promising strategy with which to elicit mucosal and systemic humoral and cellular immune responses. Additionally, new information on antigen processing and the presentation of particulate antigens by dendritic cells (DCs) has created new strategies for improved VLP vaccine candidates.

Methods for administering (introducing) viral particles directly to a mammal are generally known to those practiced in the art. For example, modes of administration include parenteral, injection, mucosal, systemic, implant, intraperitoneal, oral, intradermal, transdermal (e.g., in slow release polymers), intramuscular, intravenous including infusion and/or bolus injection, subcutaneous, topical, epidural, etc. Viral particles of the present invention can, preferably, be administered in a pharmaceutically acceptable carrier, such as saline, sterile water, Ringer's solution, and isotonic sodium chloride solution.

The dosage of a viral particle of the present invention administered to a mammal, including frequency of administration, will vary depending upon a variety of factors, including mode and route of administration; size, age, sex, health, body weight and diet of the recipient mammal; nature and extent of symptoms of the disease or disorder being treated; kind of concurrent treatment, frequency of treatment, and the effect desired.

Disclosed are expression systems comprising a packaging construct as described herein, wherein the expression system also comprises an envelope nucleic acid construct comprising an envelope glycoprotein-encoding nucleic acid sequence, wherein the envelope glycoprotein-encoding nucleic acid sequence is operably linked to at least one transcriptional control element; and also comprises a gene transfer construct comprising one or more sequences of interest. Also disclosed are expression systems, wherein an envelope glycoprotein promotes entry into a cell. Optionally, the envelope glycoprotein can be a viral envelope glycoprotein, such as the G protein of vesicular stomatitis virus (VSV-G).

Optionally, the expression system can further comprise a nuclear localization signal-encoding construct comprising a nuclear localization signal-encoding nucleic acid sequence operably linked to a tetracycline transactivator-encoding nucleic acid, such as tet-on. A nuclear localization sequence is one that directs a polypeptide from the cytoplasm to the nuclear membrane and hence the nucleus. The nuclear localization signal-encoding nucleic acid can further comprise a transcriptional control element. Transcriptional control elements are disclosed elsewhere herein. The nuclear localization signal-encoding nucleic acid sequence can also be flanked by at least one linker sequence, which can, for example, encode SEQ ID NO: 15 (GGGGS). A linker sequence can also be a generic sequence. The nuclear localization signal-encoding construct can also comprise from 5′ to 3′ a Cytomegalovirus promoter, a first linker encoding sequence, a second nuclear localization signal, a second linker sequence, and a tetracycline transactivator-encoding sequence, wherein the encoded linker is SEQ ID NO: 15 (GGGGS).

Also disclosed are expression systems comprising a first and a second packaging construct, a third nucleic acid construct, and a gene transfer construct. The first packaging construct comprises a first nucleic acid construct comprising a nucleic acid sequence that encodes a Gag protein, wherein the Gag-encoding nucleic acid sequence comprises one or more mutations that reduce frame-shifting or translational read-through and is operably linked to at least one transcriptional control element. The second packaging construct comprises a second nucleic acid construct comprising a nucleic acid sequence that encodes a Gag-Pol protein, wherein the Gag-Pol-encoding nucleic acid sequence comprises one or more mutations that reduce frame-shifting or translational read-through and is operably linked to at least one transcriptional control element. The expression system also comprises a third nucleic acid construct comprising a third nucleic acid sequence that encodes an envelope glycoprotein, wherein the third nucleic acid sequence is operably linked to at least one transcriptional control element. The expression system also comprises a gene transfer construct comprising one or more sequences of interest. Optionally, the expression system can further comprise a nuclear localization signal-encoding construct comprising a nuclear localization signal-encoding nucleic acid sequence described above operably linked to a tetracycline transactivator-encoding nucleic acid. A nuclear localization sequence is one which directs a polypeptide from the cytoplasm to the nuclear membrane and hence the nucleus.

Furthermore, the nuclear localization signal-encoding nucleic acid can further comprise a transcriptional control element. Transcriptional control elements are disclosed elsewhere herein. The nuclear localization signal-encoding nucleic acid sequence can also be flanked by at least one linker sequence, which can, for example, encode SEQ ID NO: 15 (GGGGS). The nuclear localization signal-encoding construct can also comprise from 5′ to 3′ a Cytomegalovirus promoter, a first linker encoding sequence, a second nuclear localization signal, a second linker sequence, and a tetracycline transactivator-encoding sequence, wherein the encoded linker is SEQ ID NO: 15 (GGGGS).

The expression systems disclosed above can also comprise a fourth nucleic acid construct comprising a fourth nucleic acid sequence that encodes a nuclear localization signal operably linked to a tetracycline transactivator. The fourth nucleic acid construct can further comprise a transcriptional control element, such as a promoter, for example. The nuclear localization signal-encoding sequence can also be flanked by at least one linker sequence as described above. The fourth nucleic acid sequence can also comprise a 5′ to 3′ a Cytomegalovirus promoter, a nucleic acid sequence encoding SEQ ID NO: 15 (GGGGS), a nucleic acid sequence encoding a nuclear localization signal, a nucleic acid sequence encoding SEQ ID NO: 15(GGGGS), and a nucleic acid sequence encoding a tetracycline transactivator.

Also disclosed are cell lines comprising the expression systems disclosed elsewhere herein.

Also disclosed is a gene transfer method comprising introducing into a cell a packaging nucleic acid construct described elsewhere herein, and introducing to the cell an envelope construct comprising a nucleic acid sequence that encodes an envelope glycoprotein, wherein the envelope glycoprotein encoding nucleic acid sequence is operably linked to at least one transcriptional control element and introducing into the cell a gene transfer construct described elsewhere herein comprising one or more sequences of interest; and maintaining the cell under conditions that allow formation of a virus-like particle. The virus-like particle contains the gene(s) or sequence(s) of interest.

Also disclosed is a cell comprising an exogenous sequence of interest, where the sequence of interest is transferred into the cell using the gene transfer method described above.

Also disclosed herein are methods of making a recombinant protein from a gene of interest comprising, contacting a target cell with the viral particles comprising a gene of interest as disclosed elsewhere herein, under conditions suitable to allow expression of the recombinant protein by the cell. For example, the target cell can be contacted with the viral particles in vitro or in vivo.

Also disclosed are methods of making a recombinant protein from a gene or sequence of interest comprising introducing the gene transfer constructs disclosed herein into a target cell under conditions suitable to allow expression of a recombinant protein, wherein the sequence of interest is a nucleic acid sequence encoding the recombinant protein. As disclosed elsewhere herein, the expression of the recombinant protein can be regulatable. For example, the expression of the recombinant protein can be inducible and reversible.

Also disclosed herein are methods of making a recombinant protein comprising, contacting a target cell with the viral particles comprising a gene(s) or sequence(s) of interest that encodes the recombinant protein, as disclosed elsewhere herein, under conditions suitable to allow expression of the recombinant protein by the cell. For example, the target cell can be contacted with the viral particles in vitro or in vivo.

Also disclosed herein are methods of making a recombinant protein comprising, introducing a first nucleic acid construct comprising a promoter operably linked to a regulator sequence operably linked to at least one VP16 sequence into a target cell; maintaining the cell under conditions that allow integration of the first nucleic acid sequence to integrate into the genome of the target cell and forming a modified target cell; introducing a second nucleic acid construct comprising a regulator target sequence operably linked to a sequence of interest to the modified target cell of step (b); wherein the sequence of interest is a nucleic acid sequence encoding a recombinant protein; and maintaining the modified target cell under conditions that allow expression of a recombinant protein.

Also disclosed herein are methods of making a recombinant protein comprising, introducing a first nucleic acid construct comprising a promoter operably linked to a regulator sequence operably linked to at least one VP16 sequence into a target cell; introducing a second nucleic acid construct comprising a regulator target sequence operably linked to a sequence of interest to the same target cell of step (a), wherein the sequence of interest is a nucleic acid sequence capable of encoding a recombinant protein; and maintaining the target cell under conditions that allow integration of the first and second nucleic acid sequence to integrate into the genome of the target cell and forming a modified target cell; and maintaining the modified target cell under conditions that allow expression of the recombinant protein.

For example, the first nucleic acid construct can comprise the sequence of SEQ ID NO: 44. The second nucleic acid sequence can be any of the gene transfer vectors described elsewhere herein. For example, the second nucleic acid can comprise a sequence of interest operably linked to a transcriptional control element operably linked to a regulator target sequence. The first nucleic acid construct can also comprise an IRES or IRES-like sequence. For example, the sequence of interest can be operably linked to an IRES or IRES-like sequence operably linked to a selectable marker.

Any known cell transfection technique can be employed for the method of making recombinant proteins. Other methods for contacting a cell with viral particles are disclosed elsewhere herein. Generally for in vitro methods, cells are incubated (i.e., cultured) with the constructs or vectors in an appropriate medium under suitable transfection conditions, as is well known in the art. For example, methods such as electroporation and calcium phosphate precipitation (O'Mahoney et al. (1994) DNA & Cell Biol. 13(12):1227-1232) can be used.

Also disclosed are vaccines comprising the gene transfer constructs disclosed herein. Also disclosed are methods of producing an immune response in a subject comprising administering to the subject the gene transfer constructs disclosed herein.

In addition, disclosed are methods of producing an immune response in a subject, wherein the immune response is an immune response against HIV, comprising administering to the subject the gene transfer constructs disclosed herein, wherein the sequence of interest is a sequence capable of expressing an HIV antigen.

As used herein, a “vaccine” or a “composition for vaccinating a subject” specific for a particular pathogen means a preparation, which, when administered to a subject, leads to an immunogenic response in a subject. As used herein, an “immunogenic” response is one that confers upon the subject protective immunity against the pathogen. Without wishing to be bound by theory, it is believed that an immunogenic response can arise from the generation of neutralizing antibodies (i.e., a humoral immune response) or from cytotoxic cells of the immune system (i.e., a cellular immune response) or both. As used herein, an “immunogenic antigen” is an antigen which induces an immunogenic response when it is introduced into a subject, or when it is synthesized within the cells of a host or a subject. As used herein, an “effective amount” of a vaccine or vaccinating composition is an amount which, when administered to a subject, is sufficient to confer protective immunity upon the subject. Historically, a vaccine has been understood to contain as an active principle one or more specific molecular components or structures which comprise the pathogen, especially its surface. Such structures can include surface components such as proteins, complex carbohydrates, and/or complex lipids which commonly are found in pathogenic organisms.

As used herein, however, it is to be stressed that the terms “vaccine” or “composition for vaccinating a subject” extend the conventional meaning summarized in the preceding paragraph. As used herein, these terms also relate to the sequence of interest of the instant invention or to compositions containing the sequence of interest. The sequence of interest induces the biosynthesis of one or more specified gene products encoded by the sequence of interest within the cells of the subject, wherein the gene products are specified antigens of a pathogen. The biosynthetic antigens then serve as an immunogen. As already noted, the sequence of interest, and hence the vaccine, can be any nucleic acid that encodes the specified immunogenic antigens. In a preferred embodiment of this invention, the sequence of interest of the vaccine is DNA. The sequence of interest can include a plasmid or vector incorporating additional genes or particular sequences for the convenience of the skilled worker in the fields of molecular biology, cell biology and viral immunology (See Molecular Cloning: A Laboratory Manual, 2nd Ed., Sambrook, Fritsch and Maniatis, Cold Spring Harbor Laboratory, Cold Spring Harbor, N.Y., 1989; and Current Protocols in Molecular Biology, Ausubel et al., John Wiley and Sons, New York 1987 (updated quarterly), which are incorporated herein by reference in their entirety for their teachings of examples of and the use of plasmids or vectors).

Several recombinant subunit and viral vaccines have been devised in recent years. U.S. Pat. No. 4,810,492, the contents of which is hereby incorporated by reference in its entirety for its teaching of recombinant subunit and viral vaccines, describes the production of the E glycoprotein of Japanese Encephalitis Virus (JEV) for use as the antigen in a vaccine. The corresponding DNA is cloned into an expression system in order to express the antigen protein in a suitable host cell such as E. coli, yeast, or a higher organism cell culture. U.S. Pat. No. 5,229,293, the contents of which is hereby incorporated by reference in its entirety for its teaching of methods to clone DNA into an expression system in order to express an antigen protein, discloses recombinant baculovirus harboring the gene for JEV E protein. The virus is used to infect insect cells in culture such that the E protein is produced and recovered for use as a vaccine.

U.S. Pat. No. 5,021,347 discloses a recombinant vaccinia virus genome into which the gene for JEV E protein has been incorporated. The live recombinant vaccinia virus is used as the vaccine to immunize against JEV. Recombinant vaccinia viruses and baculoviruses in which the viruses incorporate a gene for a C-terminal truncation of the E protein of dengue serotype 2, dengue serotype 4 and JEV are disclosed in U.S. Pat. No. 5,494,671. U.S. Pat. No. 5,514,375 discloses various recombinant vaccinia viruses which express portions of the JEV open reading frame extending from prM to NS2B. These pox viruses induced formation of extracellular particles that contain the processed M protein and the E protein. Two recombinant viruses encoding these JEV proteins produced high titers of neutralizing and hemagglutinin-inhibiting antibodies, and protective immunity, in mice. The extent of these effects was greater after two immunization treatments than after only one. Recombinant vaccinia virus containing genes for the prM/M and E proteins of JEV conferred protective immunity when administered to mice (Konishi et al., Virology 180: 401-410 (1991)). HeLa cells infected with recombinant vaccinia virus bearing genes for prM and E from JEV were shown to produce subviral particles (Konishi et al., Virology 188: 714-720 (1992)). Dmitriev et al. reported immunization of mice with a recombinant vaccinia virus encoding structural and certain nonstructural proteins from tick-borne encephalitis virus (J. Biotechnology 44: 97-103 (1996)). Each of these reference is hereby incorporated by reference in their entirety for their teaching of recombinant vaccinia viruses.

Recombinant virus vectors have also been prepared to serve as virus vaccines for dengue fever. Zhao et al. (J. Virol. 61: 4019-4022 (1987)) prepared recombinant vaccinia virus bearing structural proteins and NS1 from dengue serotype 4 and achieved expression after infecting mammalian cells with the recombinant virus. Similar expression was obtained using recombinant baculovirus to infect target insect cells (Zhang et al., J. Virol. 62: 3027-3031 (1988)). Bray et al. (J. Virol. 63: 2853-2856 (1989)) also reported a recombinant vaccinia dengue vaccine based on the E protein gene that confers protective immunity to mice against dengue encephalitis when challenged. Falgout et al. (J. Virol 63: 1852-1860 (1989)) and Falgout et al. (J. Virol. 64: 4356-4363 (1990)) reported similar results. Zhang et al. (J. Virol 62: 3027-3031 (1988)) showed that recombinant baculovirus encoding dengue E and NS1 proteins likewise protected mice against dengue encephalitis when challenged. Other combinations in which structural and nonstructural genes were incorporated into recombinant virus vaccines failed to produce significant immunity (Bray et al., J. Virol. 63: 2853-2856 (1989)). Also, monkeys failed to develop fully protective immunity to dengue virus challenge when immunized with recombinant baculovirus expressing the E protein (Lai et al. (1990) pp. 119-124 in F. Brown, R. M. Chancock, H. S. Ginsberg and R. Lerner (eds.) Vaccines 90: Modern approaches to new vaccines including prevention of AIDS, Cold Spring Harbor Laboratory, Cold Spring Harbor, N.Y.). Each of these references is hereby incorporated by reference in their entirety for their teaching of methods of incorporating genes into recombinant virus vaccines and examples of structural and nonstructural genes were incorporated into recombinant virus vaccines.

Immunization using recombinant DNA preparations has been reported for SLEV and dengue-2 virus, using weanling mice as the model (Phillpotts et al., Arch. Virol. 141: 743-749 (1996); Kochel et al., Vaccine 15: 547-552 (1997)). Plasmid DNA encoding the prM and E genes of SLEV provided partial protection against SLEV challenge with a single or double dose of DNA immunization. In these experiments, control mice exhibited about 25% survival and no protective antibody was detected in the DNA-immunized mice (Phillpotts et al., Arch. Virol. 141: 743-749 (1996)). In mice that received three intradermal injections of recombinant dengue-2 plasmid DNA containing prM, 100% developed anti-dengue-2 neutralizing antibodies and 92% of those receiving the corresponding E gene likewise developed neutralizing antibodies (Kochel et al., Vaccine 15: 547-552 (1997)). Challenge experiments using a two-dose schedule, however, failed to protect mice against lethal dengue-2 virus challenge. Recombinant vaccines based on the use of only certain proteins of flaviviruses, such as JEV, produced by biosynthetic expression in cell culture with subsequent purification or treatment of antigens, do not induce high antibody titers. Also, like the whole virus preparations, these vaccines carry the risk of adverse allergic reaction to antigens from the host or to the vector. Vaccine development against dengue virus and WNV is less advanced and such virus-based or recombinant protein-based vaccines face problems similar to those alluded to above. Each of these references is hereby incorporated by reference in their entirety for their teaching of methods of incorporating genes into recombinant virus vaccines and examples of structural and nonstructural genes were incorporated into recombinant virus vaccines as well as methods of immunizations using recombinant DNA preparations.

Also disclosed herein are methods for making antibodies. For example, disclosed is an in vivo method of inducing antibody production by inducing an immune response in a subject. The in vitro method comprises introducing the recombinant protein made by the methods disclosed elsewhere herein into a subject in an amount sufficient to induce an immune response. For example, the target cell can be contacted with the gene transfer constructs or viral particles disclosed herein in vitro or in vivo.

Also disclosed are methods of generating antibodies to a protein of interest comprising, (a) introducing a gene transfer construct as disclosed elsewhere herein into a target cell, wherein the transcriptional control element of the gene transfer construct is regulatable or constitutive, wherein the sequence of interest is capable of encoding a protein of interest; (b) maintaining the cell under conditions that allow integration of the nucleic acid construct in step (a) into the genome of the target cell and formation of a modified target cell; (c) introducing the modified target cell of step (b) into the subject; (d) administering to the subject an effective amount of a substance capable of regulating a transcriptional control element of the gene transfer construct in an amount sufficient to induce expression of the sequence of interest, wherein the sequence of interest is expressed in an amount sufficient to induce an immune response, and wherein the immune response generates antibodies to the protein of interest. In addition, the antibodies generated from the methods described herein can be isolated.

Also disclosed are methods of identifying an antibody that binds an antigen of interest, the method comprising, bringing into contact a sample suspected of containing antibodies that bind an antigen of interest and target cells that express the antigen of interest, and determining if an antibody in the sample binds to the antigen of interest expressed by the target cells, whereby the antibody that binds to the antigen of interest is identified as an antibody that binds the antigen of interest. Target cells that express the antigen of interest can be target cells generated by the methods described herein. The target cells used in the disclosed methods of identifying an antibody that binds an antigen of interest can be target cells that comprise the gene transfer constructs described elsewhere herein. For example, also disclosed are methods of identifying an antibody that binds an antigen of interest, the method comprising, bringing into contact a sample suspected of containing antibodies that bind an antigen of interest and target cells that express the antigen of interest, wherein the target cells comprise one or more of the nucleic acid constructs of claims 1, 55, 208, 247, and 286; and determining if an antibody in the sample binds to the antigen of interest expressed by the target cells, whereby the antibody that binds to the antigen of interest is identified as an antibody that binds the antigen of interest.

Also disclosed are methods of generating antibodies to a protein of interest comprising, (a) introducing a gene transfer construct as disclosed elsewhere herein into a target cell, wherein the transcriptional control element of the gene transfer construct is regulatable or constitutive, wherein the sequence of interest is capable of encoding a protein of interest; (b) maintaining the cell under conditions that allow integration of the nucleic acid construct in step (a) into the genome of the target cell and formation of a modified target cell; (c) introducing the modified target cell of step (b) into the subject; (d) administering to the subject an effective amount of a substance capable of regulating a transcriptional control element of the gene transfer construct in an amount sufficient to induce expression of the sequence of interest, wherein the sequence of interest is expressed in an amount sufficient to induce an immune response, and wherein the immune response generates antibodies to the protein of interest.

In addition, a control can be used in this method that does not express the antigen of interest, such that a sample suspected of containing antibodies that bind an antigen of interest that does not comprise antibodies that bind an antigen of interest, would not be identified as an antibody that binds the antigen of interest. The disclosed methods of identifying an antibody that binds an antigen of interest can also be used to identify neutralizing antibodies.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain protein of interest binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988) the contents of which is hereby incorporated by reference in its entirety for its teaching of general methods for producing antibodies and screening antibodies for specificity and activity).

Also included within the meaning of “antibody or fragments thereof” are conjugates of antibody fragments and antigen binding proteins (single chain antibodies) as described, for example, in U.S. Pat. No. 4,704,692, the contents of which is hereby incorporated by reference in its entirety for its teachings of conjugates of antibody fragments and antigen binding proteins single chain antibodies.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992) which are incorporated by reference in their entirety for their teachings of humanized antibodies).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain.

Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988), which are incorporated by reference in their entirety for their teachings of humanization of antibodies), by substituting rodent CDRs or CDR sequences for the corresponding sequences of a human antibody. Accordingly, such “humanized” antibodies are chimeric antibodies (U.S. Pat. No. 4,816,567) which is incorporated by reference in its entirety for its teachings of humanized and chimeric antibodies, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987), which are incorporated by reference in their entirety for their teachings of using a human sequence that is closest to that of the rodent as the human framework (FR) for a humanized antibody). Another method uses a particular framework derived from the consensus sequence of all human antibodies of a particular subgroup of light or heavy chains. The same framework can be used for several different humanized antibodies (Carter et al., Proc. Natl. Acad. Sci. USA, 89:4285 (1992); Presta et al., J. Immunol., 151:2623 (1993) which are also incorporated by reference in their entirety for their teachings of using a human sequence that is closest to that of the rodent as the human framework (FR) for a humanized antibody).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994 and is incorporated by reference in its entirety for its teachings of CDR residues and their influence on antigen binding).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993), which are incorporated by reference in their entirety for their teachings of the production of human antibodies upon antigen challenge). Human antibodies can also be produced in phage display libraries (Hoogenboom et al., J. Mol. Biol., 227:381 (1991); Marks et al., J. Mol. Biol., 222:581 (1991), which are incorporated by reference in their entirety for their teachings of the production of producing human antibodies in phage display libraries). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991), which are incorporated by reference in their entirety for their teachings of the preparation of preparing human monoclonal antibodies).

Also disclosed are cells that produce the monoclonal antibody. The term “monoclonal antibody” as used herein refers to an antibody obtained from a substantially homogeneous population of antibodies, i.e., the individual antibodies comprising the population are identical except for possible naturally occurring mutations that can be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984), which are hereby incorporated by reference in their entirety for their teachings of monoclonal antibodies that specifically include chimeric antibodies).

Monoclonal antibodies can also be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes can be immunized in vitro. Preferably, the immunizing agent comprises the sequence of interest or sequences of interest present in the gene transfer construct. Traditionally, the generation of monoclonal antibodies has depended on the availability of purified protein or peptides for use as the immunogen. As such, the methods disclosed herein provide a way to elicit strong immune responses and generate monoclonal antibodies by providing a large amount of the protein of interest within the viral particles that can be injected into a host animal.

The advantages to this system include ease of generation, high levels of expression, and post-translational modifications that are highly similar to those seen in mammalian systems. Use of this system involves expressing domains of a protein of interest's antibody as fusion proteins. The antigen can also be produced by inserting a gene fragment in-frame between the signal sequence and the mature protein domain of the protein of interest's antibody nucleotide sequence. This results in the display of the foreign proteins on the surface of the virion. This method allows immunization with whole virus, eliminating the need for purification of target antigens.

Generally, when making monoclonal antibodies either peripheral blood lymphocytes (“PBLs”) can be used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against the protein of interest. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further herein or in Harlow and Lane “Antibodies, A Laboratory Manual” Cold Spring Harbor Publications, New York, (1988).

After the desired hybridoma cells are identified, the clones can be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

Also disclosed are fragments of antibodies which have bioactivity. The polypeptide fragments can be recombinant proteins obtained by cloning nucleic acids encoding the polypeptide in an expression system capable of producing the polypeptide fragments thereof, such as the expression systems disclosed herein. For example, one can determine the active domain of an antibody from a specific hybridoma that can cause a biological effect associated with the interaction of the antibody with the protein of interest. For example, amino acids found to not contribute to either the activity or the binding specificity or affinity of the antibody can be deleted without a loss in the respective activity. For example, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody can be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats can be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of the monoclonal antibody or fragment thereof and one or more reagents for detecting binding of the antibody or fragment thereof to the protein of interest. The reagents can include, for example, fluorescent tags, enzymatic tags, or other tags. The reagents can also include secondary or tertiary antibodies or reagents for enzymatic reactions, wherein the enzymatic reactions produce a product that can be visualized.

Also disclosed are methods of inducing an immune response in a subject comprising introducing the recombinant protein made by the methods disclosed elsewhere herein into a subject in an amount sufficient to induce an immune response. For example, the target cell can be contacted with the viral particles in vitro or in vivo.

Also disclosed are methods of inducing an immune response in a subject comprising, (a) introducing a gene transfer construct into a target cell; (b) maintaining the cell under conditions that allow integration of the nucleic acid construct in step (a) to integrate into the genome of the target cell; and (c) introducing the target cell of step (b) into the subject in an amount sufficient to induce an immune response. For example, the sequence of interest can be capable of encoding a membrane protein (e.g.) an HIV membrane protein). In addition, expressing of the sequence of interest can be inducible, reversible, or inducible and reversible.

As used herein, an “immune response” refers to reaction of the body as a whole to the presence of an antigen which includes making antibodies, developing immunity, developing hypersensitivity to the antigen, and developing tolerance. Therefore, an immune response to an antigen also includes the development in a subject of a humoral and/or cellular immune response to the antigen of interest. A “humoral immune response” is mediated by antibodies produced by plasma cells. A “cellular immune response” is one mediated by T lymphocytes and/or other white blood cells.

As used herein, the term “antigen” refers to any agent, (e.g., any substance, compound, molecule, protein or other moiety) that is recognized by an antibody and/or can elicit an immune response in an individual.

The methods disclosed herein can be used with any cell type. In other words, any cell type can serve as the target cell for the methods disclosed herein. Eukaryotic host cells can include, but are not limited to yeast, fungi, insect, plant, animal, human and nucleated cells. Mammalian cells can also be used in conjunction with the methods described herein. A target cell can also comprise any of the nucleic acid constructs described herein. For example, target cells can comprise one or more of the gene transfer constructs and/or one or more of the packaging constructs described herein.

The terms “mammal” and “mammalian” as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and ruminents (e.g., cows, pigs, horses).

Examples of mammalian cells include human (such as HeLa cells, 293T cells, NIH 3T3 cells), bovine, ovine, porcine, murine (such as embryonic stem cells), rabbit and monkey (such as COS1 cells) cells. The cell can be a non-dividing cell (including hepatocytes, myofibers, hematopoietic stem cells, neurons) or a dividing cell. The cell can be an embryonic cell, bone marrow stem cell or other progenitor cell. Where the cell is a somatic cell, the cell can be, for example, an epithelial cell, fibroblast, smooth muscle cell, blood cell (including a hematopoietic cell, red blood cell, T-cell, B-cell, etc.), tumor cell, cardiac muscle cell, macrophage, dendritic cell, neuronal cell (e.g., a glial cell or astrocyte), or pathogen-infected cell (e.g., those infected by bacteria, viruses, virusoids, parasites, or prions).

Typically, cells isolated from a specific tissue (such as epithelium, fibroblast or hematopoietic cells) are categorized as a “cell-type.” The cells can be obtained commercially or from a depository or obtained directly from an animal, such as by biopsy. Alternatively, the cell need not be isolated at all from the animal where, for example, it is desirable to deliver the virus to the animal in gene therapy.

Although any cell type can be used, for example, to make recombinant proteins or antibodies as described elsewhere herein, the presence of oligosaccharides on the cell surface can present difficulties in crystallization and antibody development. Disclosed herein are methods of making recombinant proteins and antibodies using target cells that are defective in one or more of the enzymes involved in glycosylation of proteins, which can be used in stimulating antibody production. One such enzyme involved in glycosylation of proteins is UDP-GlcNAc:-D-mannoside-1,2-N-acetylglucosaminyltransferase I (GnTI).

Many secreted proteins, as well as integral membrane proteins of the secretory system are glycoproteins, i.e., they are modified by glycans (oligosaccharides) that are N-linked to asparagines or O-linked to serine, threonine, or hydroxyproline. N-glycosylation can be responsible for correct folding and stability of proteins, prevention of protein degradation, protein conformation and recognition, solubility of proteins, their secretion to the extracellular space, and their biological activity.

GnTI is a type II integral membrane protein, localized to medial-Golgi cisternae, which catalyzes the first step in the conversion of high mannose N-glycans into complex and hybrid structures. Complex N-glycans are critical for the viability of the developing embryo, as mice lacking a functional GnTI gene die before birth. However, complex N-glycans are not essential for viability of cells cultured in vitro as a number of mutants have been isolated which lack GnTI activity.

An example, of dealing with heterogenous N-glycans on a purified glycoprotein is to use tunicamycin treatment to eliminate all glycosylation. Thus, tunicamycin treatment along with a tetracycline-inducible expression has been used for purification of milligram quantities of non-glycosylated rhodopsin. However, this approach is not ideal because removing the N-glycans does not allow their role in the structure and function of the glycoprotein to be addressed. For example, although the precise role of glycosylation in rhodopsin structure and function is not fully understood, it clearly has an important role. Significant defects in signal transduction properties arising from the absence of glycosylation of the photoreceptor have been previously reported. Also, a rhodopsin mutant with three amino acid changes (E113Q/E134Q/M257Y) could not be purified when expressed in the presence of tunicamycin. Other cell lines that have been mutated are described in Puthalakath et al., Glycosylation Defect in Lec! Chinese Hamster Ovary Mutant is Due to a Point Mutation in N-Acetylglucosaminyltransferase I Gene, J.B.C., 271, 27818-27822 (1996), which is hereby incorporated by reference in its entirety for its teaching of cell lines that lack GnTI activity.

Another example of dealing with heterogenous N-glycans is to produce the protein in a cell which is defective in one of the various enzymes involved in N-glycan synthesis, such as GlcNAc transferase I. This approach has been used previously for isolation of a diverse collection of Chinese Hamster Ovary (CHO) cell lines resistant to various lectins resulting from deficiencies in various enzymes involved in N-glycan synthesis. Cell lines that have been mutated to generate uniform glycosylation patterns are described in US 2004/0029229, which is hereby incorporated by reference in its entirety for its teaching of cell lines that have been mutated to ensure uniform N-glycans. Reeves et al. also described cell lines that have been mutated to generate uniform glycosylation patterns (Structure and function in rhodopsin: high-level expression of rhodopsin with restricted and homogeneous N-glycosylation by a tetracycline-inducible N-acetylglucosaminyltransferase I-negative HEK293S stable mammalian cell line; PNAS 2002 Oct. 15; 99(21):13419-24. Epub 2002 Oct. 7). The GnTI gene has also been disrupted in plants as described by Koprivova et al., N-Glycosylation in the Moss Physcomitrella patens is Organized Similarly to that in Higher Plants, Plant Biology 5 (2003): 582-591, which is hereby incorporated by reference in its entirety for its teaching of cell lines that have been mutated to disrupt the gntI gene.

For example, the target cell described herein can generate a uniform glycosylation pattern on glycoproteins. The target cell optionally has reduced GnTI activity as compared to a control cell. Antisense oligonucleotides, RNAi molecules, ribozymes and siRNA molecules can be utilized to disrupt expression. Antisense oligonucleotides, RNAi molecules, ribozymes and siRNA molecules can be used alone or in combination with other therapeutic agents such as anti-viral compounds. Such methods can also be used in conjunction with the constructs and methods disclosed herein. For example, the target cell can also contain a gene transfer vector capable of expressing GnTI siRNA, wherein the expression of GnTI siRNA can be constitutive or regulatable.

Also disclosed is a method of treating a subject with a selected protein comprising administering to the subject the protein made by the methods disclosed herein. Methods of administration of the selected protein include, but are not limited to, injection (subcutaneously, epidermally, intradermally), intramucosal (such as nasal, rectal and vaginal), intraperitoneal, intravenous, oral or intramuscular. Other modes of administration include oral and pulmonary administration, suppositories, and transdermal applications. Dosage treatment can be a single dose schedule or a multiple dose schedule.

In the methods described herein, which include the administration and uptake of exogenous DNA into the cells of a subject (i.e., gene transduction or transfection), the disclosed nucleic acids can be in the form of a vector for delivering the nucleic acids to the cells, whereby the antibody-encoding DNA fragment is under the transcriptional regulation of a promoter, as would be well understood by one of ordinary skill in the art. The vector can be any of those vectors disclosed herein. Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

In one example, the recombinant retroviruses disclosed herein can be used to infect and thereby deliver to the infected cells nucleic acid encoding a broadly neutralizing antibody (or active fragment thereof).

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein in its entirety for its teaching of approaches for parenteral administration methods. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. (1995,) which is incorporated by reference herein in its entirety for its teaching of suitable formulations and various routes of administration of therapeutic compounds.

Also disclosed herein are methods of screening for an agent that modulates viral particle formation. For example, disclosed is a method of screening for an agent that modulates viral particle formation comprising introducing into a cell a packaging nucleic acid construct comprising a first and a second nucleic acid sequence, wherein the first nucleic acid sequence encodes a Gag protein, and wherein the second nucleic acid sequence encodes a Gag-Pro-Pol protein, and wherein the first and a second nucleic acid sequences comprises one or more mutations that reduce frame-shifting or translational read-through. Furthermore, the first and second nucleic acid sequences can be expressed from different coding regions of the same nucleotide sequence, and the first and second nucleic acid sequences can be operably linked to the agent to be screened. Next, an envelope construct can be introduced into the cell, and the envelope construct can comprise a third nucleic acid sequence that encodes an envelope glycoprotein, wherein the third nucleic acid sequence is operably linked to at least one transcriptional control element. The cells can then be cultured under conditions suitable to allow formation of viral particles. The viral particles can then be detected, and an increase or decrease in the number of viral particles in the presence of the agent to be screened as compared to a control indicates that the agent modulates virus particle formation. The control culture can be a separate culture or can be the same culture before or after the agent is administered. A regulator construct comprising a regulatable sequence can also be introduced into the cell, wherein the regulatable element is operably linked to at least one transcriptional control element. Various regulatable transcription control elements and regulator sequences are discussed throughout the specification. For example, the transcriptional control element can be a CMV promoter, and the regulatable element can be tetR or tetA.

Positive packaging cell transformants (i.e., cells which have taken up and integrated the retroviral vectors) can be screened for using a variety of selection markers which are well known in the art. For example, marker genes, such as green fluorescence protein (GFP), hygromycin resistance (Hyg), neomycin resistance (Neo) and β-galactosidase (β-gal) genes can be included in the constructs and assayed, using, e.g., enzymatic activity or drug resistance assays. Alternatively, cells can be assayed for reverse transcriptase (RT) activity as described by Goff et al. (1981) J. Virol. 38:239 as a measure of viral protein production.

Similar assays can be used to test for the production by packaging cells of unwanted, replication-competent helper virus. For example, marker genes, such as those described herein, can be included in the constructs also described herein. Following transient transfection of target cells with the packaging constructs disclosed herein, packaging cells (cells comprising at least the packaging constructs disclosed herein) can be subcultured with other non-packaging cells. These non-packaging cells can be infected with recombinant, replication-deficient constructs of the invention carrying the marker gene. However, because these non-packaging cells do not contain the genes necessary to produce viral particles (e.g., gag, pol and env genes), they should not, in turn, be able to infect other cells when subcultured with these other cells. If these other cells are positive for the presence of the marker gene when subcultured with the non-packaging cells, then unwanted, replication-competent virus has been produced.

Accordingly, to test for the production of unwanted helper-virus, packaging cells of the invention can be subcultured with a first cell line (e.g., NIH3T3 cells) which, in turn, is subcultured with a second cell line which is tested for the presence of a marker gene or RT activity indicating the presence of replication-competent helper retrovirus. Marker genes can be assayed for using e.g., FACS, staining and enzymatic activity assays, as is well known in the art.

Also disclosed herein are methods for making a transgenic animal. Specifically, disclosed are methods of method of making a transgenic animal comprising introducing a viral particle made by the methods disclosed herein into a zygote; allowing said zygote to develop to term; obtaining an animal whose genome comprises a nucleic acid construct capable of expressing the gene of interest; breeding said animal with a non-transgenic animal to obtain F₁ offspring and selecting an animal whose genome comprises the nucleic acid construct capable of expressing or containing a sequence of interest, wherein said animal expresses or contains the selected sequence of interest. Also disclosed are transgenic animals made by the methods disclosed herein.

The viral particles of the present invention can be introduced into the genome of an animal in order to produce transgenic, non-human animals for purposes of practicing the methods of the present invention. Selectable markers can also be used as a reporter to identify those animals comprising a sequence of interest. For example, a light-generating protein can be used as a reporter, imaging is typically carried out using an intact, living, non-human transgenic animal, for example, a living, transgenic rodent (e.g., a mouse or rat). Any technique that can be used to introduce nucleic acid into the animal cells of choice can be employed (e.g., “Transgenic Animal Technology: A Laboratory Handbook,” by Carl A. Pinkert, (Editor) First Edition, Academic Press; ISBN: 0125571658; “Manipulating the Mouse Embryo: A Laboratory Manual,” Brigid Hogan, et al., ISBN: 0879693843, Publisher: Cold Spring Harbor Laboratory Press, Pub. Date: September 1999, Second Edition, which are hereby incorporated by reference in their entirety for their teachings of techniques that can be used to introduce nucleic acids into animal cells). A variety of transformation techniques are well known in the art. Methods that can be used to introduce nucleic acid into the animal cells of choice include, but are not limited to the following.

(i) Direct Microinjection into Nuclei: Viral particles can be microinjected directly into animal cell nuclei using micropipettes to mechanically transfer the recombinant DNA. This method has the advantage of not exposing the DNA to cellular compartments other than the nucleus and of yielding stable recombinants at high frequency. See, Capecchi, M., Cell 22:479-488 (1980) which is hereby incorporated by reference in its entirety for its teachings of direct microinjection into animal cell nuclei.

For example, the viral particles can be microinjected into the early male pronucleus of a zygote as early as possible after the formation of the male pronucleus membrane, and prior to its being processed by the zygote female pronucleus. Thus, microinjection according to this method should be undertaken when the male and female pronuclei are well separated and both are located close to the cell membrane. See, e.g., U.S. Pat. No. 4,873,191 to Wagner, et al. (issued Oct. 10, 1989); and Richa, J., (2001) “Production of Transgenic Mice,” Mol. Biotech., 17:261-8 which are hereby incorporated by reference in their entirety for their teachings of direct microinjection into the early male pronucleus of a zygote.

(ii) ES Cell Transfection: The viral particles of the present invention can also be introduced into embryonic stem (“ES”) cells. ES cell clones which undergo homologous recombination with a targeting vector are identified, and ES cell-mouse chimeras are then produced. Homozygous animals are produced by mating of hemizygous chimera animals. Procedures are described in, e.g., Koller, B. H. and Smithies, O., (1992) “Altering genes in animals by gene targeting”, Ann. R. Imm 10:705-30.

(iii) Electroporation: The viral particles of the present invention can also be introduced into the animal cells by electroporation. In this technique, animal cells are electroporated in the presence of viral particles. Electrical impulses of high field strength reversibly permeabilize biomembranes allowing the introduction of the nucleic acid. The pores created during electroporation permit the uptake of macromolecules such as nucleic acids. Procedures are described in, e.g., Potter, H., et al., Proc. Nat'l. Acad. Sci. U.S.A. 81:7161-7165 (1984); and Sambrook, ch. 16 which are hereby incorporated by reference in their entirety for their teachings of introducing nucleic acids or viral particles into animal cells by electroporation.

(iv) Calcium Phosphate Precipitation: The viral particles can also be transferred into cells by other methods of direct uptake, for example, using calcium phosphate. See, e.g., Graham, F., and A. Van der Eb, Virology 52:456-467 (1973); and Sambrook, ch.16 which are hereby incorporated by reference in their entirety for their teachings of introducing nucleic acids or viral particles into animal cells by calcium phosphate precipitation.

(v) Liposomes: Encapsulation of nucleic acid within artificial membrane vesicles (liposomes) followed by fusion of the liposomes with the target cell membrane can also be used to introduce nucleic acids into animal cells. See Mannino, R. and S. Gould-Fogerite, BioTechniques, 6:682 (1988) which is hereby incorporated by reference in its entirety for its teachings of using liposomes to introduce nucleic acids into animal cells.

(vi) Transfection using Polybrene or DEAE-Dextran: These techniques are described in Sambrook, ch.16 which is hereby incorporated by reference in its entirety for its teachings of using transfection using polybrene or DEAE-Dextran to introduce nucleic acids into animal cells.

(vii) Protoplast Fusion: Protoplast fusion typically involves the fusion of bacterial protoplasts carrying high numbers of a plasmid of interest with cultured animal cells, usually mediated by treatment with polyethylene glycol. (Rassoulzadegan, M., et al., Nature, 295:257 (1982) which is hereby incorporated by reference in its entirety for its teachings of using protoplast fusion to introduce nucleic acids into animal cells).

(iix) Ballistic Penetration: Another method of introduction of nucleic acid segments is high velocity ballistic penetration by small particles with the nucleic acid either within the matrix of small beads or particles, or on the surface, Klein, et al., Nature, 327, 70-73, 1987 which is hereby incorporated by reference in its entirety for its teachings of using ballistic penetration to introduce nucleic acids into animal cells.

Electroporation has the advantage of ease and has been found to be broadly applicable, but a substantial fraction of the targeted cells may be killed during electroporation. Therefore, for sensitive cells or cells which are only obtainable in small numbers, microinjection directly into nuclei can be preferable. Also, where a high efficiency of nucleic acid incorporation is especially important, such as transformation without the use of a selectable marker (as discussed above), direct microinjection into nuclei is an advantageous method because typically 5-25% of targeted cells will have stably incorporated the microinjected nucleic acid.

Also, disclosed herein are transgenic animals comprising a sequence of interest. For example, disclosed herein are transgenic animals expressing KISS-1, FOX P3, NF κβ, micro RNA 223, or Cre recombinase.

Also disclosed are transgenic animals comprising the gene transfer constructs described herein. Also disclosed are transgenic animals made by the methods disclosed herein.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Construction of a Tetracycline-Based Single, Inducible, Reversible Lentivector

A tetracycline-based single, inducible, reversible gene transfer vector was constructed to drive the expression of a sequence of interest, eGFP. First, 1.2 kb of a human EF1-a promoter was amplified by PCR from pEF4/His (Invitrogen) and cloned into pHRCMVeGFP/blas using EcoRI and BamHI restriction enzymes. The resulting vector was designated as pHREFeGFP/blas. Next, a sequence capable of encoding a tetracycline repressor was codon optimized and linked to a SV40 nuclear localized signal. The encoded optimized tetracycline repressor gene linked to the SV40 nuclear localization signal was then cloned into pHREFeGFP/blas which replaced eGFP using NcoI and XhoI restriction enzymes. The resulting vector was designated as pHREFtet/blas. Then, 500 bps of a human CMV promoter was amplified by PCR, introducing two tet operator sequences into a 3′ CMV promoter. The PCR fragments were cloned into pHREFtet/blas using ClaI and EcoRI restriction enzymes. The resulting vector was designated as pHRCMVO2(R)EFtet/blas. The orientation of the CMV promoter was then reversed. An EGFP fragment containing bovine growth hormone polyadenylation signal was then cloned into pHRCMVO2(R)EFtet/blas, which was inturn controlled by CMV promoter. The resulting vector was designated as pHReGFPO2/EFtet/blas. Next, 1.2 kb of Human ubiquitin6 promoter was amplified by PCR from pUB6/V5-His (Invitrogen) and cloned into pHReGFPO2/EFtet/blas using EcoRI and NcoI restriction enzymes. The resulting vector was designated as pHReGFPO2/UB6tet/blas. Following this step, 1.6 kb of a CAG promoter containing 300 bps of 5′ human CMV promoter sequence and 1.2 kb of chicken β-actin promoter was obtained from pDRIVE-CAG (Invivogen) and cloned into pHReGFPO2/EFtet/blas. The pHReGFPO2/EFtet/blas was cut by SnaBI and NcoI restriction enzymes and the 5′ CMV sequence and EF promoter were removed and replaced by the CAG promoter. The resulting construct was designated as pHReGFPO2/CAGtet/blas.

### Example 2

### Generation of High Titer of Tetracycline-Based Single, Inducible, Reversible Viral Particles

293Y cells were cotransfected with packaging, envelope, and different gene transfer constructs including pHReGFPO2/EFtet/blas, pHReGFPO2/CAGtet/blas; pHReGFPO2/UB6tet/blas and pHReGFPO2/CAGtet/blas to produce different versions of inducible viral particles. The viral particle titer resulting from the contransfections was measured using fluorescent microscopy to determine eGFP expression in HeLa cells. The titers of the supernatants derived from the transfected cells was 1-4×10⁶/ml, while the titer of the concentrated supernatants (400 fold higher) was 2-10×10⁸/ml.

### Example 3

### Tightly Regulated, Inducible, Single Lentivector

Mouse T-cell lines (4×10⁴) were infected with 100 μl of the viral particle supernatants derived from pHReGFPO2/EFtet/blas (titer 2.5×10⁶/ml). On the following day, the infected cells were divided into groups: Group 1, which was incubated in media containing 0.1 μg DOX/ml, and Group 2, which was incubated in media without DOX. After the three days post-infection, the cells were analyzed by FACS analysis to determine the level of GFP expression. Analysis of Group 1 revealed the mean intensity of GFP expression signal of 16,195, which was a 44.2 fold increase in comparison with that of Group 2.

### Example 4

### Single Lentivector was Highly Sensitive to DOX and Rapidly Induced Gene Expression

To determine the DOX concentration required to induce gene expression in the single vector system, different concentration of DOX were added to cells infected with viral particles derived from pHReGFPO2/EFtet/blas (titer 2.5×10⁶/ml). GFP expression was monitored by fluorescent microscopy. FIG. 4A shows that 15 ng of DOX was sufficient to induce GFP expression within 48 hours.

### Example 5

### Constitutive Promoter Activity Significantly Affected the Inducible Promoter Activity of a Single Inducible Lentivector

To determine whether the expression level of tetracycline repressor affected the inducible ability of gene transfer vectors, different promoters were cloned into gene transfer vectors to drive the expression of tetracycline repressor. The promoters used were human EF-1a promoter (pHReGFPO2/EF-1a/blas), CAG promoter (pHReGFPO2/CAGtet/blas) and the human ubiquitin6 promoter (pHReGFPO2/UB6tet/blas). EF-1a was the strongest promoter among the three, whereas the human ubiquitin6 promoter was the weakest. Viral particles derived from the 293T cells were infected into mouse T cell lines. Positively infected cells were selected using blasticidin antibiotic. After the three days of selection, the infected cells were divided into two groups: Group 1, which was incubated in media containing DOX, and Group 2, which was incubated in media without DOX. The infected cells were analyzed by FACS analysis to measure the GFP expression after the three days in the presence or absence of DOX. Table 1 shows that the induction of the expression level of GFP using the different vectors.

TABLE 1

| Different promoter | DOX(−) | DOX(+) | Induction |
|---|---|---|---|
| EF-1α | 133 | 15,071 | 113 fold |
| CAG | 157 | 7,071 | 45 fold |
| UB6 | 263 | 4,550 | 17 fold |

The construct containing the EF-1a promoter yielded the lowest basal level of the eGFP expression, however, it also yielded the highest induction level of the eGFP expression. The induction level of the eGFP expression was over 100 fold for the EF-1a promoter construct. The human ubiquitin6 promoter yielded the highest basal level of the eGFP expression and the lowest induction level of the eGFP expression. The induction level of the eGFP expression was about 17 fold for the human ubiquitin6 promoter construct.

The effect of a constitutive promoter can be seen on two levels, first the promoters effect the basal leaking level and second, the constitutive promoter affect the maximum expression level of the gene of interest (here eGFP). The strong constitutive promoters can drive a high level of tetracycline repressor expression which facilitates and controls the basal leak level in the absence of DOX. The inducible promoter based on the CMV promoter is often less active in the T cells in comparison with other type cells such as HeLa cells.

When a strong constitutive promoter is linked to a regulator construct, for example EF1-α operably linked to tetR, is applied to the inducible system, such a strong constitutive promoter can stimulate CMV-based inducible promoter activity. When the inducible promoter operably linked to a gene of interest is additional operatively linked to a strong constitutive promoter driving expression of a regulator construct, the expression of the gene of interest becomes very active in T cells.

### Example 6

### Generation of eGFP Transgenic Mice Using a Single, Inducible Lentivector

Female mice (B6 strain) between the ages of 22 and 24 days old were superovulated with a combination of pregnant mare's serum (PMS) and human chorionic gonadotropin (HCG) as described previously. (B. Hogan, R. ÜBeddington, F. ÜCostantini, E. ÜLacy, Manipulating the Mouse Embryo (Cold Spring Harbor Laboratory Press, Cold Spring Harbor, N.Y., 1994). Donor embryos were later harvested as described by B. Hogan, R. Beddington, F. Constantini, E. Lacy, Manipulating the Mouse Embryo (Cold Spring Harbor Laboratory Press, Cold Spring Harbor, N.Y., 1994. Concentrated viral particles made using the methods described above (titer approximately 2×10⁸/ml) were delivered to single-cell stage embryos on the same day of collection using microinjection system (CellTram, Eppendorf GmbH, Hamburg, Germany). Using a micromanipulator to guide the pipette, the micropipette was pushed through the zona pellucida into the perivitelline space, and 10 pl to 100 pl of the virus stock was delivered to the embryo. The infected embryos were cultured in the KSOM-AA (Specialty Media, NJ) overnight and those two-cell stage embryos were transferred into pseudopregnant females (10-week old CD1) as described by B. Hogan, R. Beddington, F. Costantini, E. Lacy, Manipulating the Mouse Embryo (Cold Spring Harbor Laboratory Press, Cold Spring Harbor, N.Y., (1994), which is incorporated by reference herein in its entirety for its teachings of methods of making transgenic animals. 11 founders (herein referred to as EF-founders) derived from pHReGFPO2/EFtet and 8 founders (herein referred to as CAG-founders) from pHReGFPO2/CAGtet were identified.

The two versions of pHReGFPO2/EFtet and pHReGFPO2/CAGtet were generated from pHReGFPO2/Eftet/blas and pHReGFPO2/CAGtet/blas by removing the blasticidin gene to avoid the possibility of deleterious effects on the transgenic mouse. Genomic DNA was extracted from three-week old founders and analyzed by PCR and Northern Blot analysis to determine the presence of positive transgenic mice and the copy number of the integrated constructs. Table 2 shows the number of positive transgenic mice.

TABLE 2

| | # of | Rate of | Rate of |
|---|---|---|---|
| Different promoter | founder | PCR positive | single-copy |
| EF-1α | 11 | 63.6% (7/11) | 42.8% (3/7) |
| CAG | 8 | 61.5% (5/8) | 40% (2/5) |

Positive transgenic mice were identified with PCR analysis using a pair of primers targeted to tetracycline repressor gene. SEQ ID NO: 16 AND SEQ ID NO: 17 Both constructs generated a similar positive rate of transgenic mice as both constructs had approximate titers of 2×10⁸/ml. Northern analysis revealed that there were three single-copy founders in the EF-founder group, and two single-copy founders were identified in the CAG-founder group. Over half of the positive transgenic mice had two or more copies in both groups (a range from one to four). In comparison with previous reports, others had to use a titer five times higher (10×10⁸/ml) to generate founder mice that had two or more copies in both groups (a range from one to twenty). Thus, the present method provides a more efficient process.

### Example 7

### Induction of the eGFP Expression in the Transgenic Nice Using Drinking Water Containing DOX

To determine whether the inducible constructs could induce eGFP expression in vivo, the transgenic mice were fed drinking water containing 100 μg/ml DOX. GFP expression in the body (paw) and PBMCs was analyzed by fluorescent microscopy and FACS analysis before and after the transgenic mice were fed DOX. All 12 of the positive mice were able to induce the expression of eGFP in both PBMC and the body (paw), but the inducible level varied among these mice. eGFP expression was detected in all of the transgenic mice before DOX, but the level varied across the mice. eGFP expression significantly increased after the transgenic mice were fed DOX. The transgenic mice infected with the viral particles derived from the construct containing the CAG promoter yielded the highest level of induction in comparison with transgenic mice infected with the viral particles derived from the construct containing the EF-1a promoter. These data differed from the in vitro results described above.

### Example 8

### Visualization of eGFP Expression in the Body (Finger) of Transgenic Mice was Inducible and Reversible

To determine whether the transgene contained in the gene transfer constructs can be expressed throughout the entire body, a gene transfer construct as described above, comprising eGFP driven by a CAG promoter was used to generate transgenic animals as described above. With eGFP under of the control of the CAG promoter, it is possible that eGFP can be expressed throughout the entire body. To determine whether GFP was expressed throughout the entire body of transgenic animals containing the gene transfer constructs described above, fingers of the transgenic mice were analyzed by fluorescent microscopy. For this study, four of the CAG-founders described above (designated CAG-founder 1#, 2#, 6# and 7#, respectively) were chosen for analysis. Expression of eGFP was seen in CAG-founders-2# and -6# after the addition of DOX, suggesting that GFP expression in these two mice can be tightly controlled by DOX. eGFP expression in CAG-founder-1# mice revealed the brightest expression after the addition of DOX among the transgenic founders tested, while its fingers expressed eGFP at the lowest level without the DOX induction. The CAG-founder-7# exhibited some delay in expressing eGFP in response to the addition of DOX and the overall expression intensity was weak in comparison with that of other CAG-founders.

12 days after the removal of DOX from the CAG-founders, the fingers of the mice were analyzed by fluorescent microscopy again. With the exception of CAG-founder-1#, the intensity of GFP expression in the finger dramatically dropped to expression levels similar to expression levels prior to induction. The results show that the GFP expression in these transgenic mice can be inducible and reversible depending on the presence or absence of DOX.

### Example 9

### GFP Expression in the Blood Cells of the Transgenic Mice was Inducible and Reversible by DOX

GFP expression in blood cells derived from CAG-founder mice were monitored at four different time points: (1) before the mice were fed DOX in their drinking water (0.1 mg/ml DOX), (2) 12 days after the mice were fed DOX in their drinking water (0.1 mg/ml DOX), (3) 12 days after the removal of DOX from the drinking water from time point (2), and then again after the mice of time point (3) were fed DOX in their drinking water (0.1 mg/ml DOX) for 1 and 2 days. The GFP expression of the blood cells in both CAG founder 1# and CAG founder 2# mice was tightly controlled by DOX. In addition, the GFP expression could be reversed upon the withdrawal of DOX. Furthermore, the GFP expression level in the blood cells tested can be returned to the background level (level before the addition of DOX). This data indicates that the single lentivector system can induce and reverse the expression of a sequence within the gene transfer construct.

### Example 10

### Induction of GFP Expression in Multiple Organs by DOX

To determine whether the lentiviral system described above is capable of expressing a sequence of interest throughout the entire body of an animal, expression of GFP was examined. Once the CAG-founder-2# was generated, the animals were dissected and the organs were individually analyzed using fluorescent microscopy. High GFP expression was seen in the bone and muscle of the Tg mouse (CAG founder 2#), but no GFP expression was seen in the normal mouse. Observed was high GFP expression in the heart, lung, liver, kidney, spleen, and intestinal in the Tg mouse, while GFP expression in the brain of the Tg mouse was weaker. This data indicates that eGFP expression in the transgenic mice can be induced by DOX throughout the entire body, although the induction level can vary among the different organs.

### Example 11

### Determination of the Concentration of DOX in the Drinking Water Required for Inducible GFP Expression

A previous study reported that a DOX concentration of 0.1-10 mg/ml in the drinking water of transgenic mice containing a tet regulatable system, was required for inducible gene expression within the animal. To determine the concentration of DOX required for inducible expression of GFP in the transgenic mice described above, the F1 mice from CAG-founder 6# were divided into the four groups which were fed drinking water containing different concentrations of DOX including 0 ug/ml (Group 1), 4 ug/ml (Group 2), 20 ug/ml (Group 3), and 100 ug/ml (Group 4). GFP expression was monitored by visualizing GFP expression in the fingers of the transgenic mouse under UV light after 0, 1, 2, 3, 5 and 18 days of feeding the mice DOX. The intensity of the fluorescent signal in the fingers of the tested mice over the course of the experiment was observed. Group 3 and Group 4 mice began to express GFP after 1 day of DOX feeding, indicating that the DOX can rapidly induce the gene expression through drinking water. The intensity of the fluorescent signal for Groups 3 and 4 expressed their highest level of GFP expression after 5 days of DOX feeding. In addition, 4 ug/ml of DOX fed to the mice of Group 2 mice was sufficient to induce GFP expression, however the induction was delayed and the intensity was relatively weak as compared to Group 3 and 4 mice. Also of note is that the intensity of a fluorescent signal appeared in a does-dependent manner.

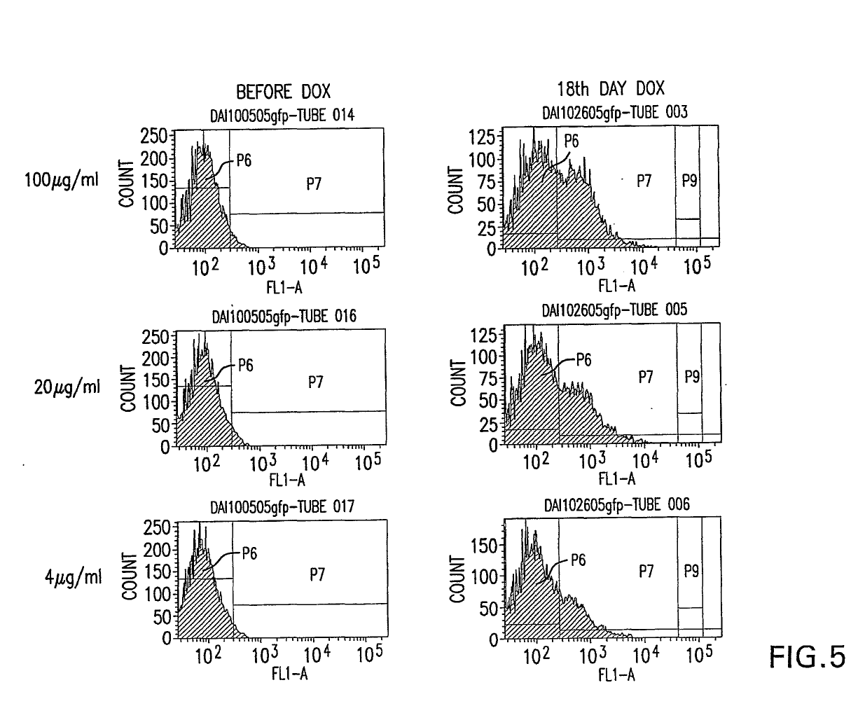

Using the FACS, positive blood cells expressing GFP were isolated and quantified. FIG. 5 shows the results of FACS analysis of GFP expression in the blood cells before and after 18 days of feeding the mice DOX. For all mice of Groups 2, 3 and 4, both the number and intensity of GFP expressing cells increased after the mice were fed DOX.

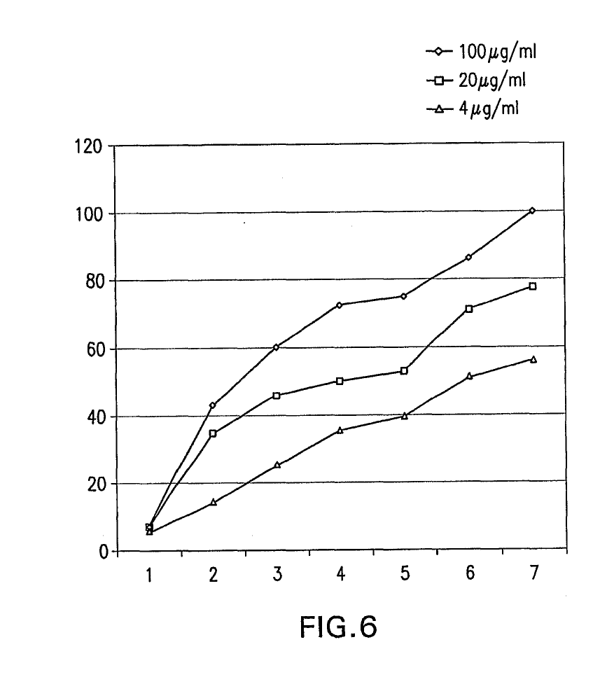

To determine the pharmacokinetics of DOX, 43% of the blood cells expressed GFP from Group 4 mice (after 18 days of DOX feeding), this level was used to set the threshold of 100 percent induction. FIG. 6 shows the induction kinetics of GFP expression in the blood cell among Group 2, 3, and 4 mice. This data reveals that DOX can induce GFP expression in the blood and fingers of the disclosed transgenic mice, and that the induction level is does-dependent.

### Example 12

### Construction of a Tetracycline-Based Single, Inducible, Reversible Lentivector to Express shRNA

The human H1 promoter was amplified from a Hela cell by PCR using a sense primer containing NotI (5′-GCGGCCGCAATTCATATT TGCATGTCGCTATGT-3′) (SEQ ID NO: 18 and an antisense primer containing one minimal 19 bps tetO sequence upstream of TATA box and another tetO sequence downstream of TATA box (5′-GAATTCGCGGATCCTCTCTATCACTGATAGGGA CTTATAA GTCTCTATCACTGATAGGGATTTCACGTTTATGGTGA-3′) (SEQ ID NO: 19). The PCR fragment containing the human H1 promoter was then cloned into pHREFtet/blas. The resulting vector was designated as pHRhH1tetOEFtet/blas.

The mouse H1 promoter was amplified from a 3T3 cell line by PCR using a sense primer containing NotI (5′-GGCGGCCGCATATGACTAGTCATGCAAATTACGCGCT-3′) (SEQ ID NO: 20) and an antisense primer containing one minimal 19 bps tetO sequence upstream of TATA box and another tetO sequence downstream of TATA box (5′-GAATTCTGGATCCTCTCTATCACTGATAGGGATTATAAGTCTCTATCACTGATAG GGATTTTACGTTTAGGGTGATTT-3′) (SEQ ID NO: 21). The PCR fragment containing the mouse H1 promoter was then cloned into pHREFtet/blas. The resulting vector was designated as pHRmH1tetOEFtet/blas.

The sequence of interest used for these experiments was shRNA designed to target the eGFP coding region (from nt 126 to 144). shRNA was generated using the sense primer (5′-GATCCAGCTGACCCTGAAGTTCATCTTCAAGAGAGATGAACTTCAGGGTCAGCT TTTTGG-3′) (SEQ ID NO: 22) and antisense primer (5′-AATTCCAAAAAGCTGACCCTGAAGTTCATCTCTCTTGAAGATGAACTTCAGGGT CAGCTG-3′) (SEQ ID NO: 23) annealed to each other and cloned into pHRhH1tetOEFtet/blas and pHRmH1tetOEFtet/blas which were previously digested with BamHI and EcoRI restriction enzymes. The resulting vectors were designated as pHRhH1GFPi(126)EFtet/blas and pHRmH1GFPi(126)EFtet/blas, respectively.

### Example 13

### Efficient Silencing of Gene Expression by Mouse H1 Inducible Promoter

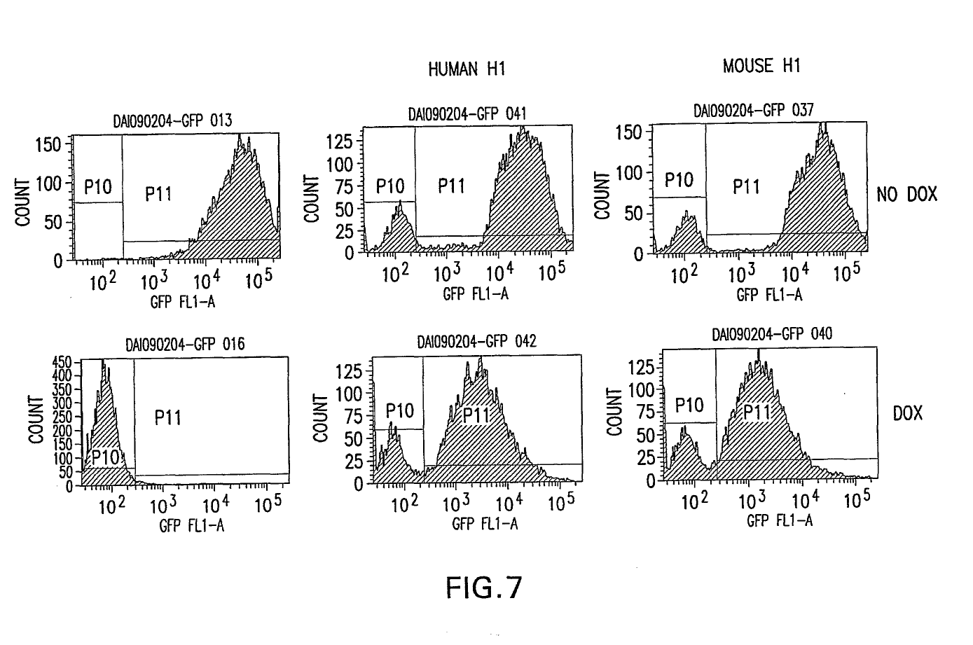

Different cell lines capable of expressing eGFP [HeLa cell, CEM-SS cell (Human T cell line) and a mouse T cell line] were infected with viral particles derived from pHRhH1GFPi(126)EFtet/blas and pHRmH1GFPi(126)EFtet/blas. After 2 days post-infection, cells containing the lentivectors were selected with an antibiotic (10 ug/ml of blasticidin) by exposing the cells to blasticidin for 3 days. Positive cells were divided into two Groups, Group 1, which were cultured in media containing 0.5 ug/ml DOX and, Group 2, which were cultured in media devoid of DOX. After the 7 days of DOX induction, cells from Groups 1 and 2 were analyzed using FACS. FIG. 7 shows that both the human and mouse H1 promoters are capable of expressing the shRNA, which inturn can efficiently silence eGFP expression in HeLa cells. The suppression of EGFP expression was up to 50 fold.

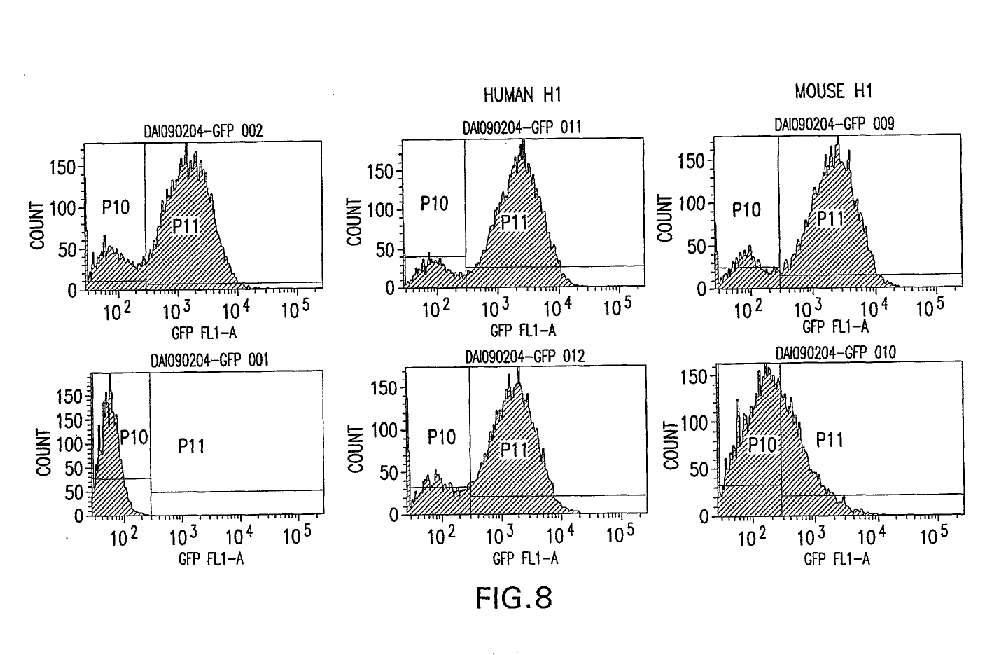

Also of note is that the human H1 promoter was less efficient in silencing eGFP expression in the Human T cell lines (1-2 fold), whereas the mouse H1 promoter reduced eGFP expression up to 10 fold (FIG. 8). In mouse T cell lines, eGFP expression was reduced to the background level by the mouse H1 promoter, while the human H1 promoter reduced eGFP expression to 4 fold. This data shows that eGFP expression levels in the cells infected with the viral particles described above is tightly controlled by DOX.

### Example 14

### Inducible Silencing of the Endogenous Protein CXCR4 by a Single Lentivector

To determine whether the single, inducible lentivector could reduce endogenous protein expression, a single lentivector comprising shRNA that targets mouse CXCR4 mRNA was constructed. The shRNA was designed to target the CXCR4 coding region (from nt 682 to 702) using sense primer (5′-GATCCAGGATGGTGGTGTTTCAATTCCTTCAAGAGA GGAATTGAAACACCACCATCCTTTTTGG-3′) (SEQ ID NO: 24) and antisense primer (5′-AATTCCAAAA AGGATGGTGGTGTTTCAATTCCTCTCTTGA AGGAATTGAAACACCACCATCCTG-3′) (SEQ ID NO: 25) which were annealed to one another and cloned into pHRmH1tetOEFtet/blas which was previously cut by BamHI and EcoRI restriction enzymes, and the blasticidin resistant marker was replaced by eGFP. The resulting vector was designated as pHRmH1GFPi(682)EFtet/GFP.

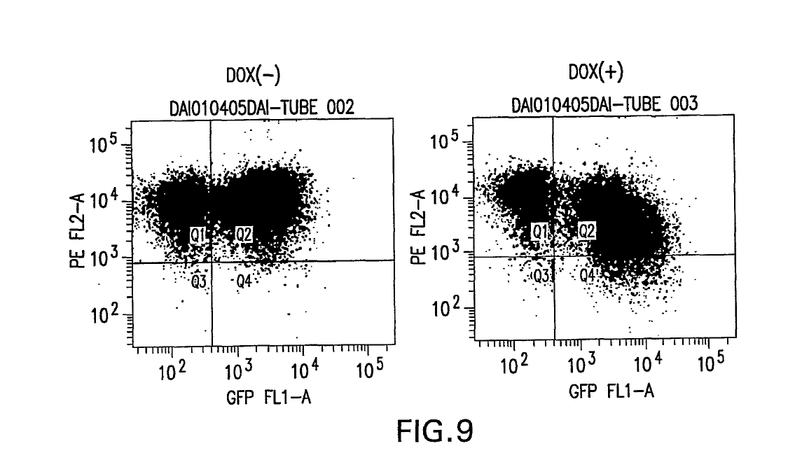

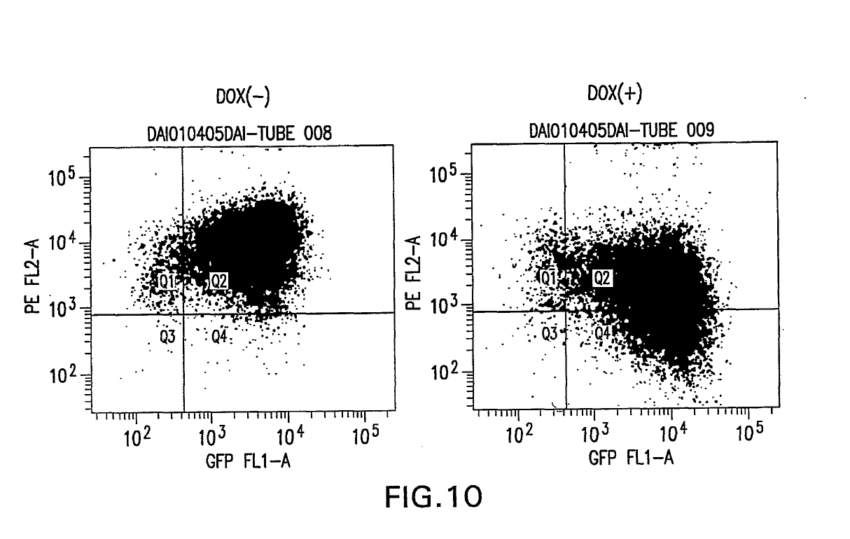

Two groups of mouse T cell lines were infected with viral particles derived from pHRmH1GFPi(682)EFtet/GFP. Group 1 was infected with a titer of 5×10⁶ IU/ml and Group 2 was infected with a titer of 5×10⁷ IU/ml. 3 days after infection, each group of cells were subdivided into two additional groups. The additional groups were either cultured in media containing 0.5 ug/ml of DOX (Group 1a and 2a) or in media without DOX (Group 1b and 2b). After 5 days of culturing, all of the cells were stained with anti-CXCR4 antibody-conjugated with PE (BD Pharmgen). These stained cells were then analyzed by FACS. Those cells infected with 5×10⁶ IU/ml of viral particles derived from pHRmH1GFPi(682)EFtet/GFP expressed GFP in 85% of the cells, while cells infected with 5×10⁷ IU/ml of the viral particles expressed GFP in 98% of the cells (FIG. 9). In the presence of DOX, the cells of Group 1a reduced the intensity of CXCR4 by 60%, while the cells of Group 2a reduced the intensity of CXCR4 by 85%. These data show that the lentivector can induce shRNA activity which can in turn reduce endogenous protein expression. In addition, these data show that the multiple copies of the integrated vector can elicit a high level of the gene silencing.

### Example 15

### A Single Lentivector can Inducibly Express shRNA to Silence Gene Expression in Transgenic Mice

To determine whether the single lentivector can express shRNA to reduce protein expression in an animal, a homogenous strain of eGFP transgenic mice from the Jackson Lab was chosen as a target. Using the homozygous strain of eGFP transgenic mice, the effect of shRNA on eGFP protein expression can be measured. To generate the lentivector for this experiment, the EF-1a promoter of the pHRmH1GFPi(126)EFtet/blas plasmid was replaced with a CAG promoter to improve the ability of the gene expression of the single lentivector in the transgenic mouse. The resulting vector was designated as pHRmH1GFPi(126)CAGtet/blas. In cell culture, the vector derived from the pHRmH1GFPi(126)CAGtet/blas, like pHRmH1GFPi(126)EFtet/blas, expressed the shRNA which was able to inducibly silence GFP expression.

2×10⁸ IU/ml of viral particles derived from pHRmH1GFPi(126)EFtet/blas or pHRmH1GFPi(126)CAGtet/blas constructs were delivered to single-cell stage embryos of the homozygous stain of GFP mice using a microinjection system (described above). The resulting transgenic mice are herein referred to as GFP/CAG-Founder mice. On the following day, two-cell stage embryos were implanted into CD1 foster mothers. Five out of the eleven mice injected with the pHRmH1GFPi(126)EFtet/blas derived viral particles were positive for the transgene as confirmed by PCR analysis, while four out of the nine mice injected with the pHRmH1GFPi(126)CAGtet/blas derived lentivector were positive for the transgene as confirmed by PCR analysis. As such, the rate transgene positive mice as deduced by PCR analysis was approximately 40% for each of the two lentivectors.

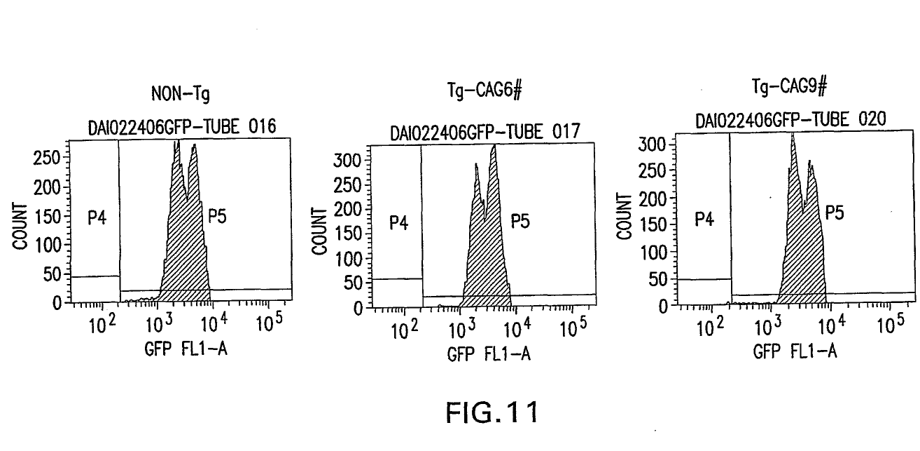

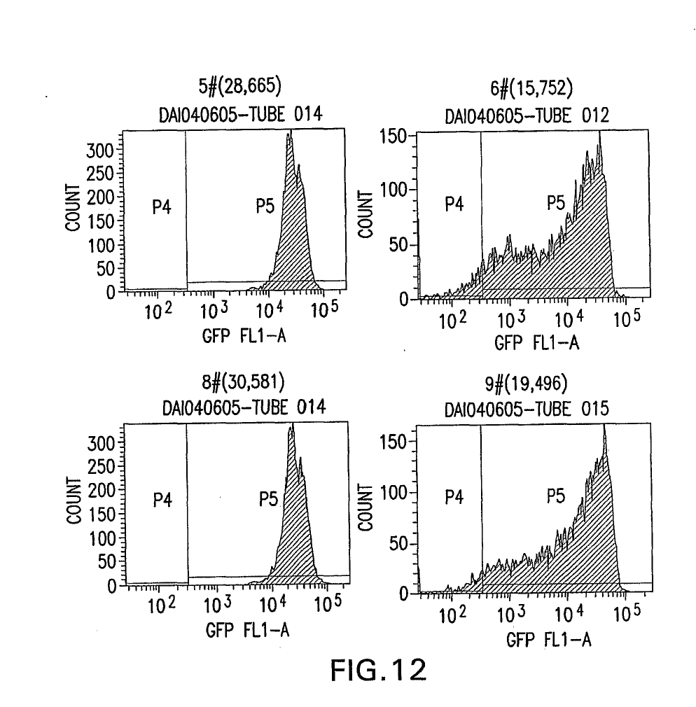

Two of the mice identified as positive for transgene expression, GFP/CAG-Founder6# and GFP/CAG-Founder9#, were raised for 4 weeks. Blood from the tail vein of 4 week old transgenic mice was then collected, and the level of GFP expression in the blood cells was analyzed by FACS analysis. The same mice were then fed DOX via their drinking water to induce expression of the shRNA. Again blood from the tail vein of 4 week old transgenic mice was collected after 5 and 10 days of DOX feeding. As before, the level of GFP expression in the blood cells was analyzed by FACS. Two of four positive transgenic mice derived pHRmH1GFPi(126)CAGtet/blas reduced the level of GFP expression after being fed DOX (See GFP/CAG-Founder6# and GFP/CAG-Founder9# in FIG. 12). The reduction of the level of GFP expression in the blood cells was not uniform, as some of the cells exhibited a reduction in the level of GFP expression up to 10 fold, whereas some of cells did not change. In addition, five out of five positive transgenic mice infected with viral particles derived from the pHRmH1GFPi(126)EFtet/blas construct, did not reveal a change in the level of GFP expression. However, the level of GFP expression for both GFP/CAG-Founder6# and GFP/CAG-Founder9# before induction was the same as the non-transgenic mice (without shRNA vector), indicating that the H1 inducible promoter in the transgenic animal can be tightly controlled by DOX (FIG. 11).

### Example 16

### A Single Lentivector can Inducibly Expresses shRNA to Silence Gene Expression in Transgenic Mice

To determine whether the single, inducible lentivector could remain functional through germline transmission, two positive transgenic mice, GFP/CAG-Founder6# (female) and GFP/CAG-Founder9# (male) were mated. All F1 mice were analyzed by Southern blot analysis to determine the number of lentivector-integrated copies. Two out of eleven F1 mice had two integrated copies of the lentivector. Other F1 mice had either one integrated copy (5 mice) or were negative (4 mice).

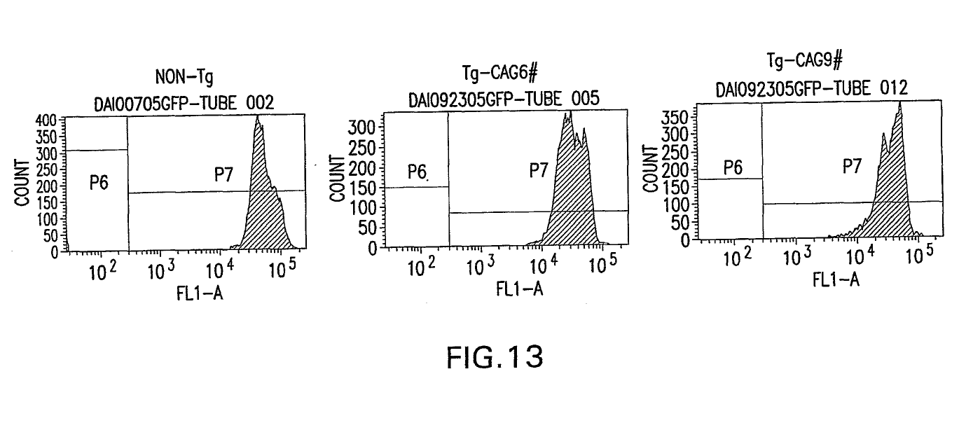

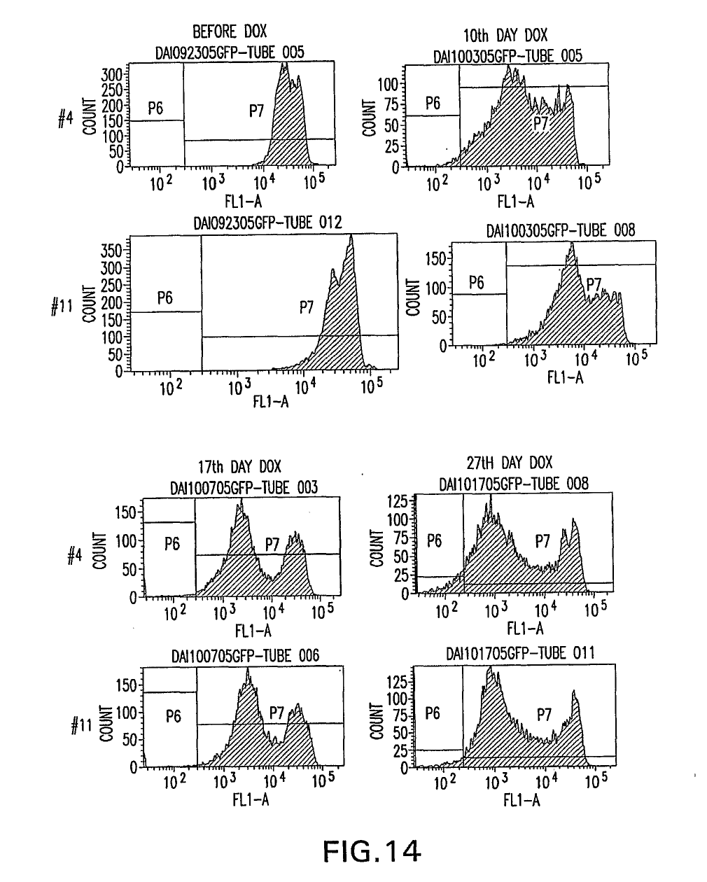

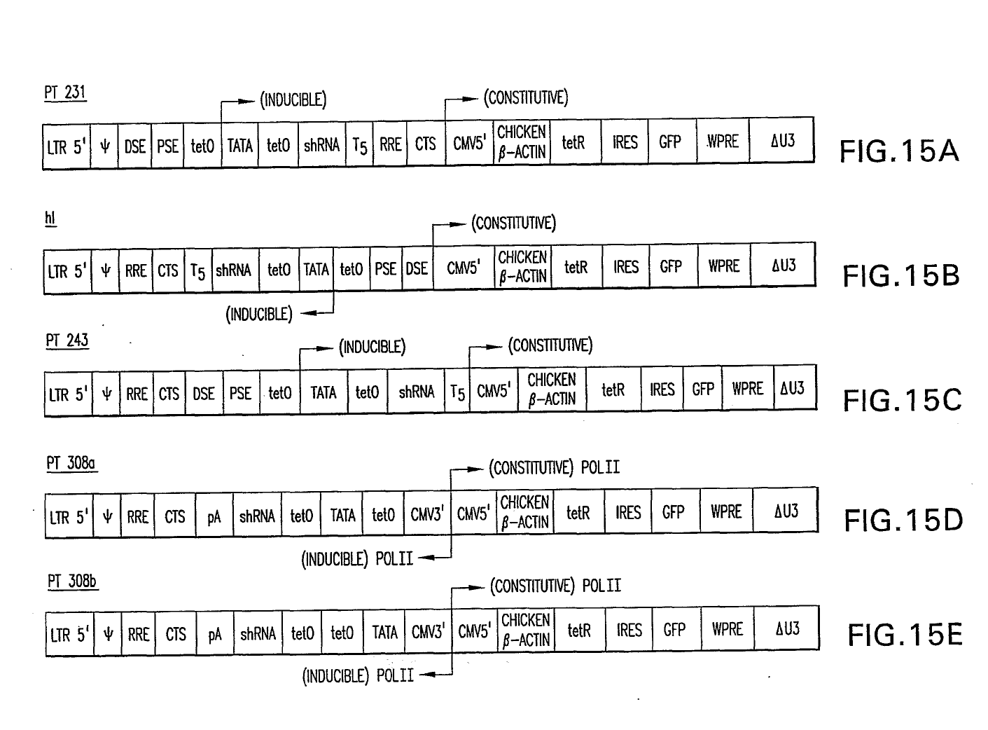

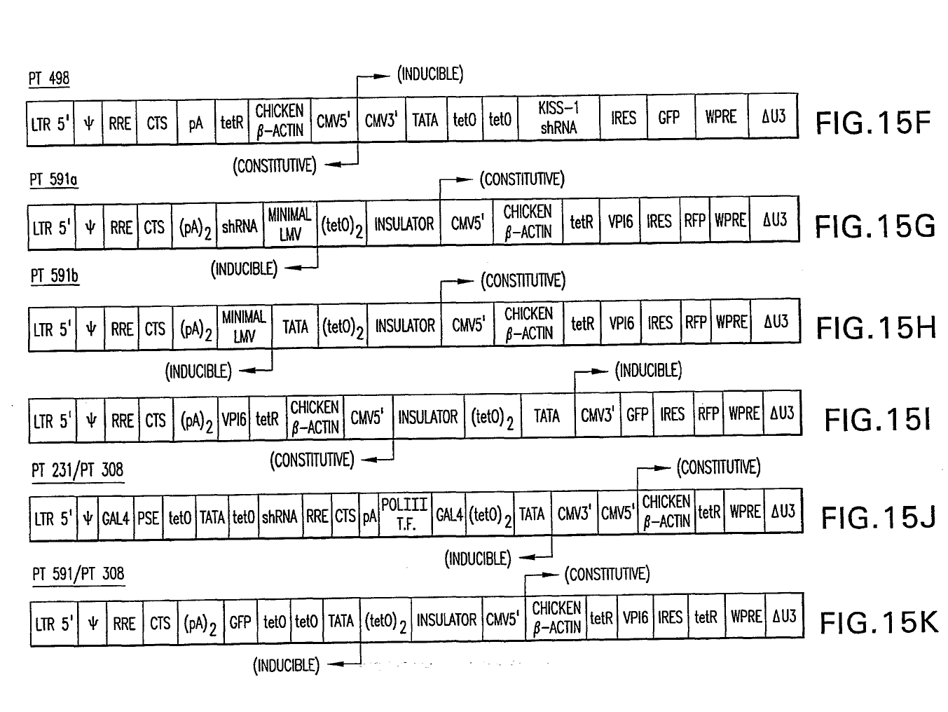

To determine whether the F1 mice containing the lentivector could inducibly reduce the level of GFP expression via DOX regulation, the F1 mice containing two integrated copies of the lentivector (F1-6# and F1-9#) were analyzed. Blood from 4 week old F1-6# and F1-9# transgenic mice was collected before the mice were fed DOX, and after 10, 17, 27 days after the mice were fed DOX. The expression level of GFP in the blood cells was analyzed by FACS analysis. FIG. 13 shows the expression level of GFP in the blood cell before the mice were fed DOX. The expression level of GFP in both transgenic mice (F1-6# and F1-9#) was similar to that of a non-transgenic mouse. FIG. 14 shows that the expression level of GFP in the transgenic mice blood cells 10, 17, 27 days after the mice were fed DOX. The after the mice were fed DOX in the blood cells decreased after the mice were fed DOX. The reduction of expression level of GFP in the blood cells was not uniform, as some of cells reduced expression level of GFP up to 30 folds, and the expression level of GFP in some of the cells remained unchanged. After 17 days post-feeding of DOX, 75% of the blood cell exhibited reduced the expression level of GFP 20 fold. After 27 days post-feeding of DOX, 85% of the blood cell exhibited reduced the expression level of GFP 30 fold. These data show that the inducible lentivector expressed the shRNA sufficiently to silence the expression of GFP in F1 mice, indicating that the single, inducible lentivector was functional after germline transmission.

### Example 17

### Single, Inducible Lentivector to Express shRNA was Functional through the Germline Transmission

To determine whether the single, inducible lentivector was functional through the germline transmission, two positive transgenic mice (GFP/CAG-Founder6# was female and GFP/CAG-Founder9# was male) mated. Using two founders to mate each other, we hope to increase the expression of shRNA in order to significantly silence the GFP level. All F1 mice were analyzed by Southern blot to determine the number of the lentivector-integrated copies. Two of eleven F1 mice had the two integrated copies of the lentivector. Others contained either one integrated copy of the lentivector (5 mice) or were negative for integration (4 mice).

To determine whether the F1 mice containing the lentivector could reduce the GFP by DOX, F1 mice containing two integrated copies of vector (F1-6# and F1-9#) were analyzed. The blood of 4 week old transgenic mice was collected before the mice were fed DOX, after the 10, 17, 27 days after the mice were fed DOX. The GFP level in the blood cell was analyzed by FACS analysis. FIG. 13 shows the GFP level in the blood cell before the DOX. The GFP expression level of both the transgenic mice (F1-6# and F1-9#) was similar to that of the non-transgenic mouse. FIG. 14 shows that the GFP level in the blood cell analyzed after the 10, 17, 27 days of DOX. The GFP level of the blood cells is less after the DOX. The reduction of the GFP level in the blood cells was not uniform, some of cells exhibited reduced GFP expression up to 30 fold, while the level of GFP expression on other cells did not change. After 17 days post feeding of DOX, 75% of the blood cells exhibited a reduced level of GFP expression of about 20 fold, while after 27 days post feeding of DOX, 85% of the blood cell exhibited a reduced level of GFP expression of about 30 fold. These data show that the inducible lentivector expressed the shRNA to silence the GFP protein in the F1 mice, indicating that the single, inducible lentivector through the germline transmission was functional.

### Example 18

### Single, Inducible Lentivector Express the Micro-RNA-Based shRNA to Silence the Gene Expression Using the Polymerase Type II

Previously, others have reported the single, inducible lentivector using tetracycline (Tet)-regulated system developed by H. Bujard and colleagues. Such a vector expresses a GFP reporter gene and a tetracycline transactivator under the control of a tetracycline-inducible promoter and a human CMV promoter in a single vector. Both the inducible constitutive promoters are arranged in the same direction from the 5′-LTR to 3′-LTR. This type of single vector expresses micro-RNA or shRNA, which is likely to hybridize to non-specific RNA sequences. These non-specific sequences can decrease the efficiency and function of the micro-RNA or shRNA.

To overcome such a problem, a single, inducible lentivector which has a bistronic, inducible promoter and a constitutive promoter that are oriented in opposite directions, was generated. To reduce promoter interference and basal level leakage of the inducible promoter, 1.2 kb of a chicken insulator was inserted between the inducible and constitutive promoter. The CAG promoter was chosen to drive expression of the tetracycline repressor gene (tetR-VP16 fusion protein) and to improve the inducible, gene expression in vivo. In addition, an improved tet-on system was used for this vector, including a mutant form tet-on called M2, and four copies of the minimal Vp16 transactivator domains replaced the single full-length Vp16 domain. Also the DsRed-exp gene was inserted downstream of the tetracycline activator gene, whose expression is driven by the CAG promoter and expressed by the IRES. The final construct was designated as pHRpATRE/CAGM2Red.

Using the Invitrogen miRNA kit, 21 bps of miRNA targeting sequence (from 480 to 500 5′-CGGCATCAAGGTGAACTTCAA-3′) (SEQ ID NO: 26) was identified to efficiently silence GFP protein expression. 157 base pairs of miRNA-GFP(480) was amplified by PCR and cloned into pHRpATRE/CAGM2Red. In addition, the DsRed-exp gene was inserted downstream of the tetracycline activator gene, whose expression is driven by the CAG promoter and expressed by the IRES. To facilitate the termination of the transcription in the inducible promoter, the double pA signal elements was introduced (pA-BGH and pA-TK). The resulting construct was designated as pHR miRNA-GFP(480)/CAGM2Red (SEQ ID NO: 37). Viral particles derived from the construct pHRmiRNA-GFP(480)/CAGM2Red were used to infect GFP expressing HeLa cells. The infected GFP expressing HeLa cells were then separated into two Groups, Group 1 was cultured in media containing 0.5 ug/ml of DOX, and Group 2, was cultured in media devoid of DOX. After the 7 days post-infection, the cells were analyzed by fluorescent microscopy. The data indicated that a PoIII-based single lentivector can express functional shRNA capable of reducing the expression of its target protein in an inducible and reversible manner.

### Example 19

### Development of a Cre-loxP-Based Conditioned, Inducible, Reversible Lentivector System

A Cre-loxp-based conditional, inducible system was generated and applied in transgenic animals. To generate this system a Cre-loxp system was combined with a tetracycline-inducible system to express a gene in a tissue-specific, inducible, reversible manner. A construct was generated by inserting 850 bps of loxp-DsRed-loxp upstream of the M2 gene in pHRpATRE/CAGM2Red, and the IRES-DsRed fragment downstream of M2 was deleted. The resulting construct was a Cre-loxp-based conditional, inducible, reversible lentivector, designated as pHRpATRE/CAGloxRedM2. Next, a miRNA-GFP(480) fragment from pHR miRNA-GFP(480)/CAGM2Red was cloned into pHRpATRE/CAGloxRedM2, thereby generating a construct designated as pHRmiRNA-GFP(480)/CAGloxRedM2. The DsRed fluorescent protein provides a means to monitor the function of Cre-loxp.

The Cre gene was amplified by PCR using the sense primer containing the Bgl II restriction enzyme and SV40 NLS which was underlined (5′-GGA AGA TCT GAA TTC ACC ATG GAT CCC AAA AAG AAA AGA AAG GTA GCA TCC AAT TTA CTA ACC GTA CAC-3′) SEQ ID NO: 39, the antisense primer containing the Xhol I restriction enzyme (5′-ATG CCG CTC GAG CTA ATC GCC ATC TTC CAG CAG GCG-3′) SEQ ID NO: 40. The PCR product was digested by the Bgl II and the Xhol I restriction enzyme, and cloned into the pHREFGFPblas using BamHI and XhoI restriction enzyme, designed as pHREF1a/CreNLS/blas. GFP expressing HeLa cells were infected with the lentivector particles derived from pHREF1a/CreNLS/blas construct to constitutively express the Cre enzyme. The infected cells were selected by blasticidin. The resulting cells are herein referred to as GFP/Cre HeLa cells. The construct viral particles derived from pHR miRNA-GFP(480)/CAGloxRedM2 were infected with or into the GFP or GFP/Cre HeLa cells. Three days after infection, the cells were divided into two groups. Group one was exposed to DOX (0.5 ug/ml) and Group two without DOX. 7 days post infection, the cells were analyzed by fluorescent microscopy. While the level of GFP expression was dramatically reduced in the GFP/Cre HeLa cells exposed to DOX in comparison with the cells that were incubated in the absence of DOX. The DsRed expression was not detected in the GFP/Cre HeLa cells, thus, the Cre enzyme can remove the Loxp-DsRed-Loxp fragment, and M2 can be conditionally expressed by Cre enzyme. In addition, these results show that M2 can induce expression of a functional shRNA to reduce the targeted protein expression in a tetracycline-controlled manner.

### Example 20

### Construction of pTREGag-HCV-Gag-Pol Packaging Construct

The tetracycline inducible promoter fragment from pTRE plasmid (purchased from Clontech) was amplified by PCR as described above. The PCR product was then cloned into pcDNA 3.1 to replace the CMV promoter to generate the tetracycline inducible plasmid, herein designed as pTRE-neo.

Next, a 1357 bps HIV-1 gag fragment containing the MA (what is this), CA and NC encoding sequences was amplified by PCR using a sense primer containing the EcoRI restriction site (5′-CGAATTCGAGCTCGGTACCCGGGATCGCGTGAAGCGCGCACGGCA AGAGGCGAG-3′) SEQ ID NO: 27 and an antisense primer containing a MscI restriction site and a 7 base mutation (5′-CATGTTGGCCAAATTTTGCCCAGGAAATTAGCCTGTCTCTCAG-3′) SEQ ID NO: 28. The 7 point mutation was introduced into the antisense primer to disturb the secondary structure (loop structure) of the PCR product which is required for framseshifting. The mutations did not change gag amino acid sequence.

Next, a 194 bp HIV-1 gag fragment containing the P2 and P6 encoding sequence was amplified by PCR using a sense primer containing MscI restriction site (5′-TTTGGCCAAGTCACAAGGGAAGGCCAG-3′) SEQ ID NO: 29 and an antisense primer containing XhoI and MluI restriction sites as well as 3 point mutations, (5′-CTCGACATGACGCGTTATTGTGACGA GGGGTCGCTGCCAAA-3′) SEQ ID NO: 30. The 3 point mutations were introduced into the sense primer to disturb the secondary structure (loop structure) of the PCR product which is required for framseshifting. The mutations did not change gag amino acid sequence. The 1357 bps of HIV-1 gag fragment was digested with EcoRI and MscI restriction enzymes. In addition, the 194 bps of HIV-1 gag fragment was digested with MscI and XhoI restriction enzymes.

The pTRE-neo vector was also digested with EcoRI and XhoI restriction enzymes. The two fragments of the PCR products were then cloned into pTRE-neo The resulting plasmid was designed as the pTRE-Gag plasmid.

Next, a 1313 bp HIV-1 gag. fragment containing the MA, CA and NC encoding sequenceS was amplified by PCR using a sense primer containing the EcoRI, MluI and BssHII restriction enzymes (5-′GAATTCACGCGTATGGGCGCGCGTGCGTCAGTA TTGAGCGGGGG-3′) SEQ ID NO: 31 and an antisense primer containing a Bgl II restriction site as well as point mutations and an additional base pair insertion (5′-CGCAGATCTTCCCTGAAGAAGTTAGCCTGTCTCTCAGTACAATC-3′) SEQ ID NO: 32 The point mutations and base pair insertion were introduced into the sense primer to disturb the secondary structure (loop structure) of the PCR product and to generate the Gag-pol fusion protein.

Then, a 3695 bp HIV-1 gag and Pol fragment containing P2, TF, protease, reverse transcriptase, integrase, vif and vpr was amplified by PCR using a sense primer containing a Bgl II restriction site (5′-AGATCTGGCATTTCCGCAGGGTAAAGCGCGTGAATTTTCCTCAGAGCAGACCAG AGCCAACA-3′) SEQ ID NO: 33 and an antisense primer containing a XhoI and a Sal I restriction (5′-GCCTCGAGCGATGTCGACACCCAATTCTGAAAAGAGTAAACAGCAG-3′) SEQ ID NO: 34. The 1313 bp PCR product was digested with EcoRI and Bgl II restriction enzymes, while the 3695 bp PCR product was digested with Bgl II and XhoI restriction enzymes.

pTRE-neo was then digested with EcoRI and XhoI restriction enzymes. The two PCR product fragments were then cloned into pTRE-neo. The resulting plasmid was designed as pTRE-Gag-Pol/dTat/dRev.

pCMV-Gag-Pol was digested by the XhoI and Sal I restriction enzyme to obtain a 1710 bp fragment containing vpr. Tat, Rev and RRE. This fragment was then cloned into the pTRE-Gag-Pol plasmid using XhoI and Sal I restriction enzymes to generate the plasmid designed as pTRE-Gag-Pol.

A 340 bp HCV IRES fragment was amplified by PCR using a sense primer containing a MluI restriction enzyme (5′-CTGACGACGCGTGCCAGCCCCCTGATGGGGCGAC-3′) SEQ ID NO: 35 and an antisense primer containing the BssH II (5′-CGCACGCGCGCCCATGGTG CGCTGTGTACGAGACCTCCCGGGGCA-3′) SEQ ID NO: 36. The PCR product was then digested with MluI and BssH II restriction enzymes, and cloned into the pTRE-Gag-Pol plasmid using MluI and BssH II restriction enzymes The resulting plasmid was designed as pTRE-HCV-Gag-Pol.

pTRE Gag was digested using MluI and XhoI restriction enzymes to obtain the a 1646 bp Gag fragment and subcloned into pTRE-HCV-Gag-Pol using MluI and XhoI restriction enzymes. The final plasmid was designed as pTREGag-HCV-Gag-Pol. The resulting plasmid lacks the conserved frameshifting loop structure. Secondly, the expression of the Gag-Pol fusion protein is regulated by HCV IRES.

### Example 21

### Generation and Analysis of KISS-1 Transgenic Mouse

Metastin is an antimetastatic peptide encoded by the KiSS-1 gene in cancer cells. Recent studies found that metastin is a ligand for the orphan G-protein-coupled receptor GPR54, which is highly expressed in specific brain regions such as the hypothalamus and parts of the hippocampus. The kisspeptins play a vital role in regulating the secretion of gonadotropin-releasing hormone (GnRH). New evidence confirms that kisspeptins acts through GPR54 to stimulate GnRH secretion. Kisspeptins and GPR54 are crucial for pubertal maturation in the primate. However, a KiSS-1 transgenic mice has not been reported until now. The experiment described below describes the production of a single, inducible lentivector-based transgenic mouse that inducibly and reversibly expresses the human KiSS-1 gene.

The human KiSS-1 gene was amplified by PCR using the sense primer containing the BamHI restriction enzyme site (5′-ATCGCGGATCCCTGCCTCTTCTCACCAA GATGAACTCACTGGT-3′) SEQ ID NO: 41, and the antisense primer containing the XhoI restriction enzyme site (5′-TTTCTCGAGTCACTGCCCCGCACCTGCGCC-3′) SEQ ID NO: 42. The PCR product was digested with BamHI and XhoI restriction enzymes, and cloned into pHRpAtetOCMVCAGtetGFP using the BamHI and XhoI restriction enzymes. The final construct was designed as pHRKiSSO2CAGtetGFP (SEQ ID NO 43).

The high titer lentivector infectious particles derived from pHRKiSSO2CAGtetGFP were used to infect single-cell stage embryos as described above. The titer of infectious particles was determined by the GFP-positive cells using the fluorescent microscopy. On the day following infection, the two-cell stage embryos were transferred into a foster mother (CD1). Positive transgenic mice were selected based on GFP expression (mice had a green body). There were 7 positive transgenic mice among the 11 mice tested. Four 4-week old founder transgenic mice (two females and two males) were fed water containing 500 ug/ml of DOX to induce the KiSS gene expression. To measure the phenotype of the KiSS transgenic mice, the vaginal opening (for the female mice) and the penises for the male mice were monitored. After five days of DOX induction, the vaginas of the 5-week old female mice were open. Additionally, the penis of the 5-week males changed in both color and size. The penises of the KiSS Tg male mice were larger in size and more developed than the penises of the control mice.

### Example 22

### Generation of GNT1-Cell Line which Express M2 Transactivated Protein Using Lentivector

A modified M2 gene (comprises tetON operably linked to VP16) was cloned into the pHREF-1 ablas vector (SEQ ID NO: 52) using BamHI and XhoI. The resulting vector was designated as PS839pHREFM2blas (SEQ ID NO: 44). An infectious particle comprising PS839pHREFM2blas was generated by cotransfection using PS839pHREFM2blas, a packaging construct (p8.91, Trono lab, Lausanne, Switzerland) and pCMV-VSV-G (pMD-G, Trono lab, Lausanne, Switzerland). The infectious particles were used to infect HEK293S cells (GnT1⁺) and GnT1⁻ HEK293S cells (the HEK293S cells (GnT1⁺) and GnT1⁻ HEK293S cells were provided by the Massachusetts Institute of Technology). The transduced cells were selected with blasticidin (20 ug/ml) over one week. The resistant cell lines were designed as GnT1⁺ HEK293S W2 cells and GnT1⁻ HEK293S W2 cells. These cell lines comprise the M2 construct described above.

### Generation of Tetracycline Induced Cell Line to Express CCR1

To date, at least ten members of the CC chemokine receptor family that have been described. The described members have been named CCR1 to CCR10 according to the IUIS/WHO Subcommittee on Chemokine Nomenclature. CCR1 was the first CC chemokine receptor identified and binds multiple inflammatory/inducible CC chemokines (for example, CCL4-6 and CCL14-16). In humans, this receptor can be found on peripheral blood lymphocytes and monocytes. This receptor is also designated cluster of differentiation marker CD191.

### Construction of a Lentiviral Vector Comprising Human CCR1

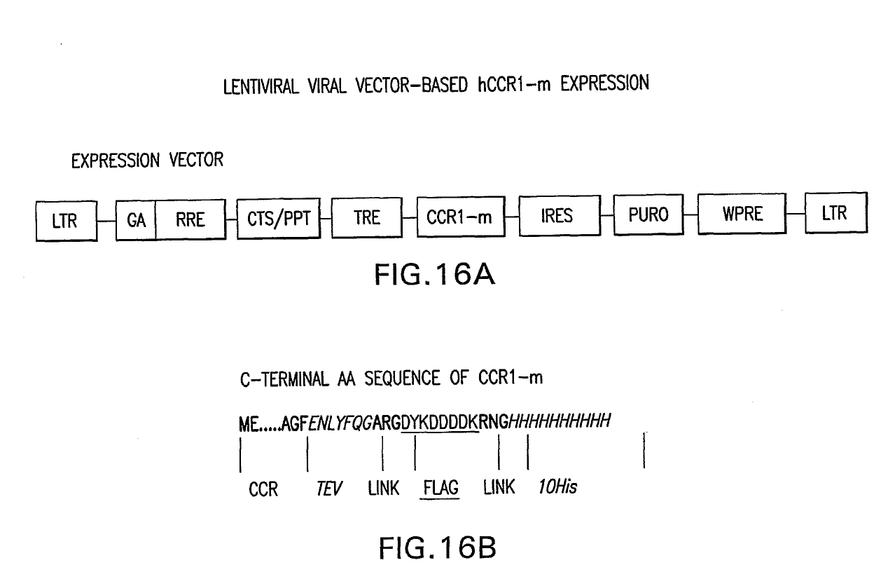

The human CCR1 cDNA (GENBANK number: BC051306) was obtained from Open Biosystems (Huntsville, Ala.) and was amplified by PCR and cloned into pCR-2.1 vector using the Invitrogen TA Clone kit (Carlsbad, Calif.). The resulting construct was designated as pCR-hCCR1. The stop codon of the CCR1 gene was mutated in order to fuse with Tag gene (TEV-Flag-10His) (see FIG. 16B). The hEP2R gene was digested with restriction enzymes and cloned into pHTRE-puro (also known as L494pHRTREpuro; SEQ ID NO: 45). The resulting vector was designated as pHTRE-hCCR1-TEV-Flag-10His (also known as PT834pHRTRE-hCCR1TEVpur; SEQ ID NO: 46). A representative map of the vector can be seen in FIG. 16A. As can be seen in FIG. 16, the promoter driving HCCR1 expression is the tet-regulatory element (TRE), followed immediately by the hCCR1 coding region. The integrated vector transcribes a bicistronic mRNA, placing the puromycin resistance gene in cis with hCCR1.

Construction of a Tetracycline-Inducible Cell Line to Express hCCR1

To generate infectious particles derived from pHTRE-hCCR1-TEV-Flag-10His, the pHTRE-hCCR1-TEV-Flag-10His plasmid was cotransfected with the p8.91 packaging construct (Trono lab, Lausanne, Switzerland) and pCMV-VSV-G (pMD-G; Trono lab, Lausanne, Switzerland) into 293T cells. The viral particles comprising pHTRE-hCCR1-TEV-Flag-10His were used to infect GnT1⁻ HEK293S W2 cells that have reduced GnTI activity that also express a tetracycline transactivator. The infected cell was found to produce a high level of CFTR protein (3-5 mg/10⁹ cells), which is 1 log higher than other known systems used for expression of membrane proteins. The transduced cells were selected by puromycin (from 1.0 to 4.0 ug/ml) to establish the stable cell line designated as hCCR1-mCell.

Analysis of hCCR1 Expression in Tetracycline-Inducible Cell Line

The hCCR1-mCell (which was selected with 0, 1, 2 and 4 ug puromycin/ml) was grown in a 6-well plate and 1 ug/ml of DOX was added to the culture medium. The next day the induced hCCR1-mCell was harvested and analyzed by Western blot using a primary antibody (M2 flag antibody) and a second antibody (HRP-conjugated anti-mouse antibody). The blot was also co-stained by anti-tubulin to serve as a control. A 52 kDa band was detected by the M2 flag antibody in the hCCR1-mCell, but not from the HERK cell, while a 55 kDa band was detected in all cells.

### High Level of Surface Expression CCR1

To determine whether the anti-CCR1 specific antibody detect the cell surface expressin of CCR1 in hCCR1-mCell, Alexa Fluor® 647 conjugated mouse anti-human CCR1 monoclonal antibody (CAT#557914, BD Bioscience, San Jose, Calif.) was used to stain hCCR1-mCells both with or without the induction with DOX (24 hours). As control a non-transduced 293 cell line was included. The results showed that the DOX induced cell line expressed a very high cell surface level of CCR1. The induction level was about 10 fold in the induced hCCR1-mCells in comparison with non-induced hCCR1-mCells.

Induction of CCR1 to Stop Cell Growth and/or to Cause Apoptosis

The hCCR1-mCell was grown on the 6-well plate in the presence or absence of 1 ug/ml of DOX. Cell growth was monitored by microscopy. After 24 hours of induction with DOX, the hCCR1-mCells stopped growing, while the majority of the induced hCCR1-mCells detached from the plate. The data indicates that the high level of hCCR1 expression caused the activation of the hCCR1 signal without the ligand.

### Example 23

### Generation of Tetracycline-Induced Cell Line to Express CFTR (Cl⁻ Anion Transmembrane Channel)

### Construction of CFTR His(10×) Lentiviral Vector.

The CFTR His(6×) lentiviral vector (also known as PT764pHRTRECFTR-His6puro; SEQ ID NO: 47) DNA was digested with BstXI and XhoI to remove the C-terminal 6×His-containing DNA fragment. Using wild-type CFTR plasmid DNA as template, PCR was used to amplify a 10×His-containing C-terminal CFTR BstXI/XhoI DNA fragment. After digestion with BstXI and XhoI, the fragment was cloned and confirmed by endonuclease restriction and nucleotide sequence analysis. The resulting vector was designated as CFTR His(10×) (also known as PT823pHRTRECFTR-His10pur; SEQ ID NO: 48).

Transduction of GnT1⁺ and ⁻HEK293S (GnT1⁻) (Cells with CFTR His(6×) and CFTR His(10×) Lentiviral Vectors.

Each of the lentiviral vectors were packaged, as described elsewhere herein and used to transduce GnT1⁺ HEK293S W2 cells and GnT1⁻ HEK293S W2 cells. After two days of supplementing the medium with 25 ug of puromycin per ml, cell lines that highly expressed CFTR were selected. After four days of selection, the surviving cells were expanded in medium without puromycin.

### Immunoblot Analysis.

Three million cells of each type (transduced GnT1⁺ HEK293S W2 cells and GnT1⁻ HEK293S W2 cells) were collected and the membrane fraction was prepared for immunoblot analysis. Additionally, a CFTR⁺ control (CFTR-FLAG) was analyzed. The blotted proteins were detected with the R1104 anti-CFTR MAb. The results showed expression of both the 6× and the 10× tagged CFTR proteins in the transduced GnT1⁻ HEK293S W2 cells. It was observed that the migration of the band from the in the transduced GnT1⁻ HEK293S W2 cells in the polyacrylamide gel, was faster than the band from the same protein expressed in the transduced GnT1⁺ HEK293S W2 cells.

### Analysis of Ion Channel Function in GnT1⁻ HEK293S Cells Expressing CFTR-His(10×).

To determine whether the expressed CFTR-His-tag(10×) protein is active, halide efflux was measured in GnT1⁺ and GnT1⁻ cell lines with the halide-quenched dye 6-methoxy-N-(3-sulfopropyl)quinolinium (SPQ, Molecular Probes). For comparison, GnT1⁺ cells expressing wild-type CFTR was analyzed in parallel. Briefly, transduced HEK293s wt-CFTR, HEK293S CFTR-His(10×), HEK293S GnT1⁻ CFTR-His(10×) cell lines were seeded on glass cover slips, and grown until ˜50% confluent. The cells were then hypotonically loaded with 10 mM SPQ for 10 min and placed in a quenching NaI buffer. Fluorescence of single cells was measured with a Zeiss inverted microscope, a PTI imaging system, and a Hamamatsu camera. Excitation was at 340 nm, and emission was measured at 410 nm. Cells were bathed in a quenching buffer (NaI) at the beginning of the experiments and were switched after establishment of a stable baseline to a halide-free dequenching buffer at 200 seconds. Cells were stimulated with agonist (20 μM Forskolin) at 620 seconds and then returned to the quenching NaI buffer. Fluorescence was normalized for each cell to its baseline value, and change in fluorescence was shown as a percent increase above basal fluorescence. The mean of the total number (at least thirty) of cell analyzed at each time point was plotted. The results obtained demonstrate significant activation of halide efflux for each of the cell lines. The HEK293S CFTR-His(10×), and the GnT1⁻ HEK293S CFTR-His(10×) cell lines both generated greater changes in fluorescence compared to HEK293S wt-CFTR.

### Example 24

### Generation of a Tetracycline-Induced Cell Line to Express Human EP2R

### Construction of a Lentiviral Vector Comprising EP2

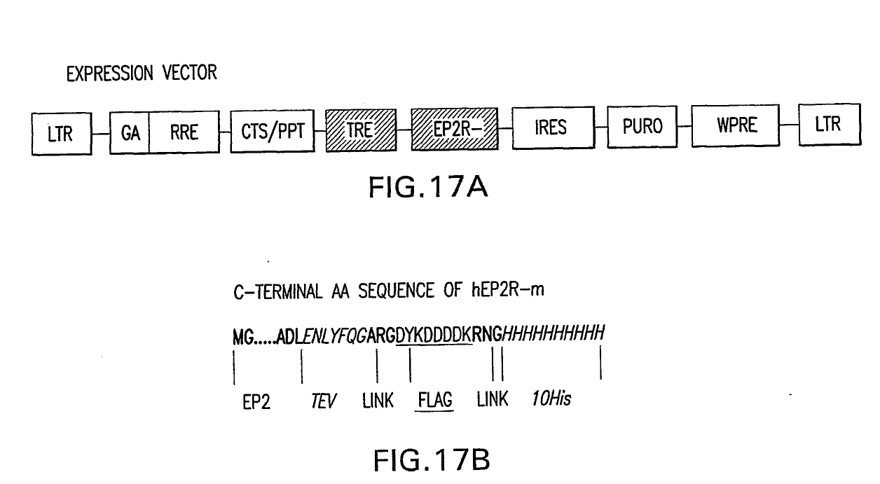

hEP2 cDNA was obtained from Schering Ag (Berlin, Germany) and was amplified by PCR and cloned into pCR-2.1 vector using Invitrogen TA Clone kit (Carlsbad, Calif.). The resulting construct was designated as pCR-hEP2R (SEQ ID NO: 49) The stop codon of the EP2 gene was mutated in order to fuse with Tag gene (TEV-Flag-10His) (FIG. 17). The hEP2R gene was digested with restriction enzymes and cloned into pHTRE-puro (SEQ ID NO: 45). The resulting vector was designated as pHTRE-hEP2R-TEV-Flag-10His (SEQ ID NO: 50). A representative map of the vector can be seen in FIG. 17. As can be seen in FIG. 17, the promoter driving hEP2R expression is the tet-regulatory element (TRE), followed immediately by the hEP2R coding region. The integrated vector transcribes a bicistronic mRNA, placing the puromycin resistance gene in cis with hEP2R.

Construction of a Tetracycline-Inducible Cell Line to Express hEP2R

To generate the infectious particle derived from pHTRE-hEP2R-TEV-Flag-10His (SEQ ID NO: 50), the pHTRE-hEP2R-TEV-Flag-10His plasmid (SEQ ID NO: 50) was cotransfected with the p8.91 packaging construct (Trono lab, Lausanne, Switzerland) and pCMV-VSV-G (pMD-G; Trono lab, Lausanne, Switzerland) into 293T cells. The viral particles comprising pHTRE-hEP2R-TEV-Flag-10His were used to infect a genetically-modified cell line that has reduced GnTI activity (GnT1⁻ HEK293S W2 cells), that also express a tetracycline transactivator. The infected cell was found to produce a high level of hEP2R protein (3-5 mg/10⁹ cells), which is 1 log higher than other known systems used for expression of membrane proteins. The transduced cells were selected by puromycin (from 1.0 to 4.0 ug/ml) to establish the stable cell line designated as hEP2R-mCell.

Analysis of hEP2R Expression in Tetracycline-Inducible Cell Line

The hEP2R-mCell (which was selected with 0 ug or 2 ug puromycin/ml) was grown in a 6-well plate and 1 ug/ml of DOX was added to the culture medium. The next day the induced hEP2R-mCell was harvested and analyzed by Western blot using the primary antibody (M2 flag antibody) and a second antibody (HRP-conjugated anti-mouse antibody). The blot was co-stained by anti-tubulin to serve as a control. Three bands were detected by the M2 flag antibody in the hEP2R-mCell, but not the HERK cell. The size of three bands was from 45 to 53 kDa, which were smaller than that of tubulin (55 kDa). The size of hEP2R was expected at 53 kDa.

Induction of hEP2R to Stop Cell Growth and/or to Cause Apoptosis

The hEP2R-mCell was grown in a 6-well plate in the presence or absence of 1 ug/ml of DOX. Cell growth was monitored by the microscopy. After 24 hours of induction, the hEP2R-mCell stop growing, while the majority of the induced hEP2R-mCell detached from the plate. The data indicates that the high level of hEP2R expression caused the activation of the hEP2R signal without the ligand.

1-452. (canceled)

453. A transgenic animal expressing a sequence of interest, wherein the sequence of interest is selected from the group consisting of Kiss-1, FOX P3, NF κβ micro RNA 223, and Cre.

454. A method of making a transgenic animal, comprising:
a) Introducing a single nucleic acid construct to a zygote;
b) allowing said zygote to develop to term;
c) obtaining an animal whose genome comprises the nucleic acid construct;
d) breeding said animal with a non-transgenic animal to obtain F1 offspring;
e) selecting an animal whose genome comprises the nucleic acid construct;
wherein the single nucleic acid construct comprises a vector, wherein the vector is selected from the group consisting of (i) a vector comprising a first nucleic acid sequence, a second nucleic acid sequence, and a third nucleic acid sequence, wherein the first nucleic acid sequence comprises a sequence of interest operably linked to a first transcriptional control element,
wherein the second nucleic acid sequence is operably linked to a second transcriptional control element and encodes a polypeptide that controls the expression of the first nucleic acid sequence, wherein the third nucleic acid sequence comprises a regulator target sequence operably linked to the first transcriptional control element, and wherein the first and second transcriptional control elements are oriented in opposite directions; and (ii) a vector comprising a first nucleic acid sequence, a second nucleic acid sequence, and a third nucleic acid sequence, wherein the first nucleic acid sequence, the second nucleic acid sequence, and the third nucleic acid sequence are operably linked to single transcriptional control element, wherein the first nucleic acid sequence comprises a sequence of interest, wherein the second nucleic acid sequence encodes a polypeptide that is capable of controlling the expression of the first nucleic acid sequence, wherein the third nucleic acid sequence comprises a regulator target sequence operably linked to the first transcriptional control element, and wherein the first transcriptional control element is capable of driving expression of the first and second nucleic acid sequences.

455. The method of claim 454, wherein the regulator target sequence of the single nucleic acid construct comprises at least one tet operator sequence

456. The method of claim 454, wherein the regulator target sequence of the single nucleic acid construct comprises a TATA box flanked by two tet operator sequences.

457. The method of claim 454, wherein the regulator target sequence of the single nucleic acid construct comprises the sequence of SEQ ID NO: 6.

458. The method of claim 454, wherein the second nucleic acid sequence of the single nucleic acid construct comprises a tetracycline repressor-encoding. nucleic acid sequence.

459. The method of claim 454, wherein the second nucleic acid sequence of the single nucleic acid construct comprises the sequence of SEQ ID NO: 1.

460. The method of claim 454, wherein the second nucleic acid sequence of the single nucleic acid construct comprises a tetracycline activator-encoding nucleic acid sequence.

461. The method of claim 454, wherein expression of the first nucleic acid sequence of the single nucleic acid construct is regulatable.

462. A transgenic animal made by the method of claim 454.
